(19) United States
(12) Patent Application Publication
(10) Pub. No.: US 2020/0029563 A1
(43) Pub. Date: Jan. 30, 2020

(54) NOVEL 5-SUBSTITUTED IMIDAZOLYLMETHYLDIOXOLANE DERIVATIVES

(71) Applicants: Bayer CropScience Aktiengesellschaft, Monheim am Rhein (DE); Bayer Aktiengesellschaft, Leverkusen (DE)

(72) Inventors: Pierre-Yves COQUERON, Lyon (FR); David BERNIER, Lyon (FR); Pierre GENIX, Lyon (FR); Ricarda MILLER, Duesseldorf (DE); Sebastien NAUD, Lyon (FR); Sven WITTROCK, Berlin (DE); Stephane BRUNET, Saint Andre De Corcy (FR); Philippe KENNEL, Biot (FR); Ruth MEISSNER, Leverkusen (DE); Ulrike WACHENDORFF-NEUMANN, Neuwied (DE); Andreas GOERTZ, Dormagen (DE)

(21) Appl. No.: 16/337,089

(22) PCT Filed: Sep. 22, 2017
(86) PCT No.: PCT/EP2017/074055

§ 371 Date: Mar. 27, 2019

(30) Foreign Application Priority Data
Sep. 29, 2016 (EP) 16191285.2

(51) Int. Cl. A01N 43/50; C07D 405/06; C07F 7/08; C07D 405/14

(52) U.S. Cl. CPC A01N 43/50; C07D 405/14; C07F 7/0812; C07D 405/06

(57) ABSTRACT

The present invention relates to novel 5-substituted imidazolylmethyldioxolane derivatives, to processes for preparing these compounds, to compositions and mixtures comprising these compounds, and to the use thereof as biologically active compounds, especially for control of harmful microorganisms in crop protection and in the protection of materials and as plant growth regulators.

The present invention relates to novel 5-substituted imidazolylmethyldioxolane derivatives, to processes for preparing these compounds, to compositions comprising these compounds, and to the use thereof as biologically active compounds, especially for control of harmful microorganisms in crop protection and in the protection of materials and as plant growth regulators.

It is already known that imidazole derivatives, which may be substituted at the imidazole ring, and salts thereof can be used in crop protection as fungicides, safeners and/or plant growth regulators (cf. e.g. WO-A 2013/076228, U.S. Pat. No. 4,085,209, WO-A 2014/118170, EP-A 2 746 259, U.S. Pat. Nos. 4,118,461, 4,115,578, DE-A 2604047, DE-A 2750031, Manabe, Akio; Kirino, Osamu; Funaki, Yuji; Hisada, Yoshio; Takano, Hirotaka; Tanaka, Shizuya, Agricultural and Biological Chemistry (1986), 50(12), 3215-17, JP-A 60069067, EP-A 0 130 366, NL-A 8201572, DE-A 2935452, and DE-A 2732750). EP-A 0029355 discloses certain 1-[2-(4-diphenypethyl]-1H-azolylketals, their preparation and use for combatting microorganisms harmful to plants, especially phytopathogenic fungi. The respective azolyl moiety is non-substituted. Also EP-A 0065485 discloses certain azolyl ketals, their preparation and use in crop protection and pharmaceutical products. Again, the respective azolyl moiety is non-substituted. From EP-A 0363582 certain azolyl dioxolanes and their use as microbicides are known. Also in this case, the respective azolyl moiety is non-substituted. Moreover, WO-A 2013/036866 and DE-A 1940388 disclose certain imidazolylmethyldioxolane derivatives which are useful in the pharmaceutical field.

Since the ecological and economic demands made on modern active ingredients, for example fungicides, are increasing constantly, for example with respect to activity spectrum, toxicity, selectivity, application rate, formation of residues and favourable manufacture, and there can also be problems, for example, with resistances, there is a constant need to develop novel fungicidal compounds and compositions which have advantages over the known compounds and compositions at least in some areas.

Accordingly, the present invention provides novel compounds of formula (I)

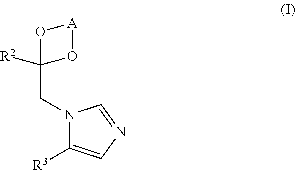

wherein
A represents a linear C₁-C₆-alkylene bridge which may be substituted by 1, 2 or up to the maximum possible number of identical or different groups R¹, wherein
R¹ represents halogen, C₁-C₆-alkyl, C₂-C₆-alkenyl, C₂-C₆-alkynyl, C₃-C₈-cycloalkyl, C₃-C₈-cycloalkyl-C₁-C₄-alkyl, C₁-C₆-alkoxy, C₁-C₆-alkylthio, phenyl, phenyl-C₁-C₄-alkyl, phenyl-C₂-C₄-alkenyl or phenyl-C₂-C₄-alkynyl;
   wherein the aliphatic moieties, excluding cycloalkyl moieties, of R¹ may carry 1, 2, 3 or up to the maximum possible number of identical or different groups W, wherein
   each R^{a} independently of one another is selected from halogen, CN, nitro, phenyl, C₁-C₄-alkoxy and C₁-C₄-haloalkoxy; wherein the phenyl may be substituted by 1, 2, 3, 4 or 5 substituents selected independently of one another from halogen, CN, nitro, C₁-C₄-alkyl, C₁-C₄-alkoxy, C₁-C₄-haloalkyl, C₁-C₄-haloalkoxy;
   and wherein the cycloalkyl and/or phenyl moieties of R¹ may carry 1, 2, 3, 4, 5 or up to the maximum number of identical or different groups R^{b}, wherein
   each R^{b} independently of one another is selected from halogen, CN, nitro, C₁-C₄-alkyl, C₁-C₄-alkoxy, C₁-C₄-haloalkyl and C₁-C₄-haloalkoxy;
   or two radicals R¹ bound on two adjacent carbon atoms, together with the carbon atoms to which they are bound, form a 3-, 4-, 5-, 6- or 7-membered saturated or unsaturated carbocyclic ring or a 3-, 4-, 5-, 6- or 7-membered saturated or unsaturated heterocyclic ring containing 1, 2, or 3 identical or different heteroatoms selected from O, S and N as ring members, where the carbocyclic or heterocyclic ring may carry 1, 2 or 3 substituents selected independently of one another from halogen, CN, nitro, C₁-C₄-alkyl, C₁-C₄-alkoxy, C₁-C₄-haloalkyl, and C₁-C₄-haloalkoxy;
R² represents naphthyl, 5-membered heteroaryl, or a substituent of formula Q,
   wherein
   the naphthyl and 5-membered heteroaryl is non-substituted or substituted by one or more group(s) selected from halogen, cyano, sulfanyl, pentafluoro-λ⁶-sulfanyl, C₁-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-haloalkenyl, C₂-C₈-alkynyl, C₂-C₈-haloalkynyl, C₂-C₈-alkenyloxy, C₂-C₈-haloalkenyloxy, C₃-C₈-alkynyloxy, C₃-C₈-haloalkynyloxy, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, benzyloxy, phenoxy, 4-halogen-substituted phenoxy, 4-(C₁-C₈-haloalkyl)-substituted phenoxy, benzylsulfanyl, phenylsulfanyl, or 6-membered heteroaryloxy, which is non-substituted or substituted by one or more group(s) selected from halogen and C₁-C₈-haloalkyl; and
   wherein Q represents a 6-membered aromatic cycle of formula (Q-I)

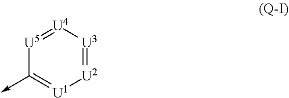

wherein
U¹ represents CX¹ or N;
U² represents CX² or N;
U³ represents CX³ or N;
U⁴ represents CX⁴ or N;
U⁵ represents CX⁵ or N;
   wherein X¹, X², X³, X⁴, and X⁵ independently from each other represent hydrogen, halogen, nitro, cyano, sulfanyl, pentafluoro-λ⁶-sulfanyl, C₁-C₈-haloalkyl having 1 to 5 halogen atoms, C₃-C₈-cycloalkyl, C₃-C₇-halocycloalkyl having 1 to 5 halogen atoms, C₃-C₇-cycloalkenyl, C₂-C₈-alkenyl, C₂-C₈-alkynyl, C₁-C₈-alkoxy, C₁-C₈-haloalkoxy having 1 to 5 halogen atoms, C₁-C₈-alkylsulfenyl, C₂-C₈-alkenyloxy, C₃-C₈-alkynyloxy, C₃-C₆-cycloalkoxy, C₁-C₈-alkylsulfinyl, C₁-C₈-alkylsulfonyl, tri(C₁-C₈-alkyl)-silyoxy, tri(C₁-C₈-alkyl)-silyl, aryl, aryloxy, arylsulfenyl, heteroaryl, heteroaryloxy,
   wherein the aryl, aryloxy, arylsulfenyl, heteroaryl, heteroaryloxy is non-substituted or substituted by one or more group(s) selected from halogen, cyano, sulfanyl, pentafluoro-λ⁶-sulfanyl, C₁-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-alkynyl, C₂-C₈-alkenyloxy, C₂-C₈-haloalkenyloxy, C₃-C₈-alkynyloxy, C₃-C₈-haloalkynyloxy, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, 6-membered heteroaryloxy, benzyloxy, phenyloxy, benzylsulfanyl, or phenylsulfanyl,
   wherein the benzyl, phenyl, 5-membered heteroaryl, 6-membered heteroaryl, 6-membered heteroaryloxy, benzyloxy, phenyloxy, benzylsulfanyl or phenylsulfanyl is non-substituted or substituted by one or more group(s) selected from halogen, CN, nitro, C₁-C₄-alkyl, C₁-C₈-haloalkyl, C₁-C₄-alkoxy, C₁-C₄-haloalkoxy or pentafluoro-λ⁶-sulfanyl;
and wherein at most two of U¹, U², U³, U⁴ or U⁵ can represent N;
or
U¹ and U² or U² and U³ or U³ and U⁴ form together an additional saturated or unsaturated 4 to 6-membered halogen- or C₁-C₈-alkyl-substituted or non-substituted ring;
R³ represents halogen, hydroxyl, cyano, isocyano, nitro, amino, sulfanyl, pentafluoro-λ⁶-sulfanyl, carboxaldehyde, hydroxycarbonyl, C₂-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-alkynyl, C₂-C₈-alkenyloxy, C₂-C₈-haloalkenyloxy, C₃-C₈-alkynyloxy, C₃-C₈-haloalkynyloxy, C₁-C₈-alkylamino, C₁-C₈-haloalkylamino, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylcarbonyl, C₁-C₈-haloalkylcarbonyl, arylcarbonyl, aryl-C₁-C₆-alkylcarbonyl, C₃-C₈-cycloalkylcarbonyl, C₃-C₈-halocycloalkylcarbonyl, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, aminothiocarbonyl, C₁-C₈-alkoxycarbonyl, C₁-C₈-haloalkoxycarbonyl, C₃-C₈-cycloalkoxycarbonyl, C₂-C₈-alkoxyalkylcarbonyl, C₂-C₈-haloalkoxyalkylcarbonyl, C₃-C₁₀-cycloallcoxyalkylcarbonyl, C₁-C₈-alkylaminocarbonyl, di-C₁-C₈-alkylaminocarbonyl, C₃-C₈-cycloalkylaminocarbonyl, C₁-C₈-alkylcarbonyloxy, C₁-C₈-haloalkylcarbonyloxy, C₃-C₈-cycloalkylcarbonyloxy, C₁-C₈-alkylcarbonylamino, C₁-C₈-haloalkylcarbonylamino, C₁-C₈-alkylaminocarbonyloxy, di-C₁-C₈-alkylaminocarbonyloxy, C₁-C₈-alkyloxycarbonyloxy, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy , C₁-C₈-haloalkylsulfonyloxy, C₁-C₈-alkylaminosulfamoyl, di-C₁-C₈-alkylaminosulfamoyl, (C₁-C₈-alkoxyimino)-C₁-C₈-alkyl, (C₃-C₇-cycloalkoxyimino)-C₁-C₈-alkyl, hydroxyimino-C₁-C₈-alkyl, (C₁-C₈-alkoxyimino)-C₃-C₇-cycloalkyl, hydroxyimino-C₃-C₇-cycloalkyl, (C₁-C₈-alkylimino)-oxy, (C₁-C₈-alkylimino)-oxy-C₁-C₈-alkyl, (C₃-C₇-cycloalkylimino)-oxy-C₁-C₈-alkyl, (C₁-C₆-alkylimino)-oxy-C₃-C₇-cycloalkyl, (C₁-C₈-alkenyloxyimino)-C₁-C₈-alkyl, (C₁-C₈-alkynyloxyimino)-C₁-C₈-alkyl, (benzyloxyimino)-C₁-C₈-alkyl, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, benzyloxy, phenyloxy, benzylsulfanyl, benzylamino, phenylsulfanyl, or phenylamino, wherein the benzyl, phenyl, 5-membered heteroaryl, 6-membered heteroaryl, benzyloxy or phenyloxy is non-substituted or substituted by one or more group(s) selected from halogen, hydroxyl, cyano, isocyano, amino, sulfanyl, pentafluoro-λ⁶-sulfanyl, carboxaldehyde, hydroxycarbonyl, C₁-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-alkynyl, C₂-C₈-alkenyloxy, C₂-C₈-haloalkenyloxy, C₃-C₈-alkynyloxy, C₃-C₈-haloalkynyloxy, C₁-C₈-alkylamino, C₁-C₈-haloalkylamino, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylcarbonyl, C₁-C₈-haloalkylcarbonyl, arylcarbonyl, aryl-C₁-C₆-alkylcarbonyl, C₃-C₈-cycloalkylcarbonyl, C₃-C₈-halocycloalkylcarbonyl, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, aminothiocarbonyl, C₁-C₈-alkoxycarbonyl, C₁-C₈-haloalkoxycarbonyl, C₃-C₈-cycloalkoxycarbonyl, C₂-C₈-alkoxyalkylcarbonyl, C₂-C₈-haloalkoxyalkylcarbonyl, C₃-C₁₀-cycloalkoxyalkylcarbonyl, C₁-C₈-alkylaminocarbonyl, di-C₁-C₈-alkylaminocarbonyl, C₃-C₈-cycloalkylaminocarbonyl, C₁-C₈-alkylcarbonyloxy, C₁-C₈-haloalkylcarbonyloxy, C₃-C₈-cycloalkylcarbonyloxy, C₁-C₈-alkylcarbonylamino, C₁-C₈-haloalkylcarbonylamino, C₁-C₈-alkylaminocarbonyloxy, di-C₁-C₈-alkylaminocarbonyloxy, C₁-C₈-alkyloxycarbonyloxy, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkylaminosulfamoyl, di-C₁-C₈-alkylaminosulfamoyl, (C₁-C₈-alkoxyimino)-C₁-C₈-alkyl, (C₃-C₇-cycloalkoxyimino)-C₁-C₈-alkyl, hydroxyimino-C₁-C₈-alkyl, (C₁-C₈-alkoxyimino)-C₃-C₇-cycloalkyl, hydroxyimino-C₃-C ₇-cycloalkyl, (C₁-C₈-alkylimino)-oxy, (C₁-C₈-alkylimino)-oxy-C₁-C₈-alkyl, (C₃-C₇-cycloalkylimino)-oxy-C₁-C₈-alkyl, (C₁-C₆-alkylimino)-oxy-C₃-C₇-cycloalkyl, (C₁-C₈-alkenyloxyimino)-C₁-C₈-alkyl, (C₁-C₈-alkynyloxyimino)-C₁-C₈-alkyl, (benzyloxyimino)-C₁-C₈-alkyl, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, benzyloxy, phenyloxy, benzylsulfanyl, benzylamino, phenylsulfanyl, or phenylamino;

and its salts or N-oxides.

The salts or N-oxides of the compounds of formula (I) also have fungicidal properties.

The formula (I) provides a general definition of the imidazole derivatives according to the invention. Preferred radical definitions for the formulae shown above and below are given below. These definitions apply to the end products of the formulae (I), (I-1), (I-1-Q-I-1), (I-1-Q-I-2) and (I-1-Q-I-3) and likewise to all intermediates.
A preferably represents a linear C₁-C₅-alkylene bridge which may be substituted by 1, 2 or up to the maximum possible number of identical or different groups R¹.
A more preferably represents a linear C₂-C₅-alkylene bridge which may be substituted by 1, 2 or up to the maximum possible number of identical or different groups R¹.
A more preferably represents a linear C₂- or C₃-alkylene bridge which may be substituted by 1, 2 or up to the maximum possible number of identical or different groups R¹.
A even more preferably represents an ethylene bridge which may be substituted by 1 or 2 identical or different groups R¹.
R¹ preferably represents halogen, C₁-C₄-alkyl, C₂-C₆-alkenyl, C₂-C₆-alkynyl, C₁-C₄-alkoxy, C₁-C₄-alkylthio, cyclopropyl, phenyl, benzyl, phenylethenyl or phenylethinyl,
   wherein the aliphatic moieties, excluding the cycloalkyl moieties, of R¹ may carry 1, 2, 3 or up to the maximum possible number of identical or different groups R^{a} which independently of one another are selected from
   R^{a} halogen, CN, nitro, phenyl, C₁-C₄-alkoxy and C₁-C₄-haloalkoxy; wherein the phenyl may be substituted by 1, 2, 3, 4 or 5 substituents selected independently of one another from halogen, CN, nitro, C₁-C₄-alkyl, C₁-C₄-alkoxy, C₁-C₄-haloalkyl, C₁-C₄-haloalkoxy;
   wherein the cycloalkyl and/or phenyl moieties of R¹ may carry 1, 2, 3, 4, 5 or up to the maximum number of identical or different groups R^{b} which independently of one another are selected from
   R^{b} halogen, CN, nitro, C₁-C₄-alkyl, C₁-C₄-alkoxy, C₁-C₄-haloalkyl and C₁-C₄-haloalkoxy.
R¹ more preferably represents fluoro, chloro, bromo, iodo, methyl, ethyl, propyl, isopropyl, butyl, methoxy, ethoxy, cyclopropyl, CF₃, allyl, CH₂C≡C—CH₃ or CH₂C≡CH,
   wherein the aliphatic groups R¹ may carry 1, 2, 3 or up to the maximum possible number of identical or different groups R^{a} which independently of one another are selected from
   R^{a} halogen, CN, nitro, phenyl, C₁-C₄-alkoxy and C₁-C₄-haloalkoxy; wherein the phenyl may be substituted by 1, 2, 3, 4 or 5 substituents selected from halogen, CN, nitro, C₁-C₄-alkyl, C₁-C₄-alkoxy, C₁-C₄-haloalkyl, C₁-C₄-haloalkoxy.
R¹ more preferably represents fluoro, chloro, bromo, iodo, methyl, ethyl, propyl, isopropyl, butyl, methoxy, ethoxy, methoxymethoxy, cyclopropyl, CF₃, allyl, CH₂C≡C—CH₃ or CH₂C≡CH.
R¹ even more preferably represents methyl, ethyl, n-propyl or CF₃.
R¹ represents in one preferred embodiment methyl.
R¹ represents in another preferred embodiment ethyl.
R¹ represents in a further preferred embodiment n-propyl.
R¹ represents in a further preferred embodiment CF₃.
A more preferably represents a linear C₂- or C₃-alkylene bridge which may be substituted by 1, 2 or up to the maximum possible number of identical or different groups R¹, wherein each R¹ is independently selected from C₁-C₄-alkyl, C₁-C₄-haloalkyl, C₁-C₄-alkoxy, C₁-C₄-alkoxy-C₁-C₄-alkoxy and C₁-C₄-haloalkoxy, and preferably from methyl, ethyl, n-propyl, CF₃, methoxy, ethoxy and methoxymethoxy, or two substituents R¹ bound on adjacent carbon atoms, together with the carbon atoms to which they are bound, form a cyclopentyl or cyclohexyl ring.
A more preferably represents a linear C₂- or C₃-alkylene bridge which may be substituted by 1 or 2 group(s) R¹, wherein each R¹ is independently from each other selected from C₁-C₄-alkyl and C₁-C₄-haloalkyl, preferably from methyl, ethyl, n-propyl and CF₃.
A more preferably represents a linear C₂-alkylene bridge which may be substituted by 1 or 2 group(s) R¹, wherein each R¹ is independently from each other selected from methyl, ethyl, n-propyl and CF₃.
A most preferably represents ethylene, 1,2-propylene, 1,2-butylene, 2,3-butylene or 1,2-pentylene, in particular ethylene or 1,2-propylene.
R² preferably represents naphthyl, thiazolyl, thienyl or a substituent of formula Q, more preferably naphthyl, 1,3-thiazol-5-yl, 1,3-thiazol-4-yl, 2-thienyl, 3-thienyl or a substituent of formula Q,
   wherein
   the naphthyl, thiazolyl, thienyl, 1,3-thiazol-5-yl, 1,3-thiazol-4-yl, 2-thienyl, 3-thienyl is non-substituted or substituted by one or more group(s) selected from halogen, cyano, sulfanyl, pentafluoro-λ⁶-sulfanyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-haloalkenyl, C₂-C₈-alkynyl, C₂-C₈-haloalkynyl, C₂-C₈-alkenyloxy, C₂-C₈-haloalkenyloxy, C₃-C₈-alkynyloxy, C₃-C₈-haloalkynyloxy, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, benzyloxy, phenoxy, 4-halogen-substituted phenoxy, 4-(C₁-C₈-haloalkyl)-substituted phenoxy, benzylsulfanyl, phenylsulfanyl, or 6-membered heteroaryloxy, which is non-substituted or substituted by one or more group(s) selected from halogen and C₁-C₈-haloalkyl; preferably is non-substituted or substituted by one or more group(s) selected from halogen, cyano, pentafluoro-λ⁶-sulfanyl, C₁-C₈-alkyl, C₁-C₈-haloalkyl, C₁-C₈-alkyloxy, C₁-C₈-haloalkyloxy, C₃-C₇-cycloalkyl, C₃-C₇-halocycloalkyl, C₂-C₈-alkenyl, C₂-C₈-alkynyl, C₁-C₈-alkylsulfanyl, C₁-C₈-haloalkylsulfanyl, benzyl, phenyl, 5-membered heteroaryl, 6-membered heteroaryl, benzyloxy, phenoxy, 4-halogen-substituted phenoxy, 4-(C₁-C₈-haloalkyl)-substituted phenoxy, benzylsulfanyl, phenylsulfanyl or 6-membered heteroaryloxy, which is non-substituted or substituted by one or more group(s) selected from halogen and C₁-C₈-haloalkyl, more preferably is non-substituted or substituted by one or more group(s) selected from halogen, pentafluoro-λ⁶-sulfanyl, C₁-C₈-alkyl, C₁-C₈-haloalkyl, C₁-C₈-alkyloxy, C₁-C₈-haloalkyloxy, C₁-C₈-haloalkylsulfanyl, phenyl, 5-membered heteroaryl, 6-membered heteroaryl, phenoxy, 4-halogen-substituted phenoxy, 4-(C₁-C₈-haloalkyl)-substituted phenoxy, phenylsulfanyl or pyridinyloxy, which is non-substituted or substituted by one or more group(s) selected from halogen and C₁-C₄-haloalkyl, more preferably is non-substituted or substituted by one or more group(s) selected from fluorine, chlorine, bromine, iodine, pentafluoro-λ⁶-sulfanyl, methyl, ethyl, n-propyl, isopropyl, n-, iso-, sec-, tert-butyl, difluoromethyl, trifluoromethyl, methoxy, trifluoromethoxy, chlorodifluoromethoxy, 1,1,2,2-tetrafluoroethoxy, methylsulfanyl, trifluoromethylsulfanyl, phenyl, pyridinyloxy, phenoxy, 4-fluorophenoxy, 4-chlorophenoxy, 4-bromophenoxy, 4-iodophenoxy, 4-(trifluoromethyl)-substituted phenoxy, or phenylsulfanyl, most preferably is substituted by one or more, preferably by one group(s) selected from 4-fluorophenoxy, 4-chlorophenoxy, 4-bromophenoxy, 4-iodophenoxy, and 4-(trifluoromethyl)-substituted phenoxy.
Q preferably represents a 6-membered aromatic cycle of formula (Q-I)

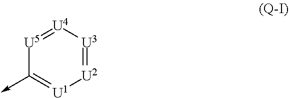

wherein U¹, U², U³, U⁴ or U⁵ are defined as outlined above and X¹, X², X³, X⁴ and X⁵ have the preferred, more preferred or most preferred meaning given below.
X¹, X², X³, X⁴ and X⁵ in the definitions for U¹, U², U³, U⁴ or U⁵ preferably represent independently from each other hydrogen, halogen, pentafluoro-λ⁶-sulfanyl, C₁-C₈-haloalkyl having 1 to 5 halogen atoms, C₁-C₈-alkoxy, C₁-C₈-haloalkoxy having 1 to 5 halogen atoms, C₁-C₈-alkylsulfanyl, C₃-C₈-cycloalkyl, C₃-C₇-halocycloalkyl having 1 to 5 halogen atoms, C₃-C₈-alkynyloxy, C₃-C₆-cycloalkoxy, aryloxy, and heteroaryloxy,
   wherein the aryloxy, and heteroaryloxy is non-substituted or substituted by one or more group(s) selected from halogen, cyano, sulfanyl, pentafluoro-λ⁶-sulfanyl, C₁-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-alkynyl, C₂-C₈-alkenyloxy, C₂-C₈-haloalkenyloxy, C₃-C₈-alkynyloxy, C₃-C₈-haloalkynyloxy, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, 6-membered heteroaryloxy, benzyloxy, phenyloxy, benzylsulfanyl, or phenylsulfanyl, preferably is non-substituted or substituted by one or more group(s) selected from halogen, pentafluoro-λ⁶-sulfanyl, C₁-C₈-haloalkyl, and C₁-C₈-haloalkyloxy.
X¹, X², X³, X⁴ and X⁵ in the definitions for U¹, U², U³, U⁴ or U⁵ more preferably represent independently from each other hydrogen, halogen, pentafluoro-λ⁶-sulfanyl, C₁-C₈-alkyl, C₁-C₈-haloalkyl having 1 to 5 halogen atoms, C₁-C₈-alkoxy, C₁-C₈-haloalkoxy having 1 to 5 halogen atoms, C₁-C₈-alkylsulfanyl, C₃-C₈-cycloalkyl, C₃-C₇-halocycloalkyl having 1 to 5 halogen atoms, C₃-C₈-alkynyloxy, C₃-C₆-cycloalkoxy, phenyloxy, and pyridinyloxy,
   wherein the phenyloxy, and pyridinyloxy is non-substituted or substituted by one or more group(s) selected from halogen, pentafluoro-λ⁶-sulfanyl, C₁-C₈-haloalkyl, and C₁-C₈-haloalkyloxy, preferably is non-substituted or substituted by one or more group(s) selected from halogen, pentafluoro-λ⁶-sulfanyl and C₁-C₄-haloalkyl, more preferably is non-substituted or substituted by one or more group(s) selected from fluorine, chlorine, bromine, iodine, pentafluoro-λ⁶-sulfanyl, difluoromethyl, trifluoromethyl.
X¹, X², X³, X⁴ and X⁵ in the definitions for U¹, U², U³, U⁴ or U⁵ more preferably represent independently from each other hydrogen, fluorine, chlorine, bromine, iodine, pentafluoro-λ⁶-sulfanyl, methyl, ethyl, n-propyl, isopropyl, n-, iso-, sec-, tert-butyl, difluoromethyl, trifluoromethyl, cyclopropyl, fluorocyclopropyl, chlorocyclopropyl, methoxy, trifluoromethoxy, chlorodifluoromethoxy, 1,1,2,2-tetrafluoroethoxy, methylsulfanyl, propargyloxy, cyclohexyloxy, phenyloxy, and pyridinyloxy,
   wherein the phenyloxy, and pyridinyloxy is non-substituted or substituted by one or more group(s) selected from halogen, pentafluoro-λ⁶-sulfanyl, C₁-C₈-haloalkyl, and C₁-C₈-haloalkyloxy, preferably is non-substituted or substituted by one or more group(s) selected from halogen, pentafluoro-λ⁶-sulfanyl and C₁-C₄-haloalkyl, more preferably is non-substituted or substituted by one or more group(s) selected from fluorine, chlorine, bromine, iodine, pentafluoro-λ⁶-sulfanyl, difluoromethyl, trifluoromethyl.
X¹, X², X³, X⁴ and X⁵ in the definitions for U¹, U², U³, U⁴ or U⁵ more preferably represent independently from each other hydrogen, fluorine, chlorine, bromine, iodine, pentafluoro-λ⁶-sulfanyl, methyl, ethyl, n-propyl, isopropyl, n-, iso-, sec-, tert-butyl, difluoromethyl, trifluoromethyl, cyclopropyl, fluorocyclopropyl, chlorocyclopropyl, methoxy, trifluoromethoxy, chlorodifluoromethoxy, 1,1,2,2-tetrafluoroethoxy, methylsulfanyl, propargyloxy, cyclohexyloxy, phenyloxy and pyridin-3-yloxy,
   wherein the phenyloxy and pyridin-3-yloxy is non-substituted or substituted by one or more group(s) selected from fluorine, chlorine, bromine, iodine, pentafluoro-λ⁶-sulfanyl, difluoromethyl, trifluoromethyl.
X¹ more preferably represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, most preferably represents hydrogen, fluorine, chlorine, difluoromethyl or trifluoromethyl.
X² more preferably represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, most preferably represents hydrogen.
X³ more preferably represents hydrogen, fluorine, pentafluoro-λ⁶-sulfanyl, methyl, ethyl, n-propyl, isopropyl, n-, iso-, sec-, tert-butyl, difluoromethyl, trifluoromethyl, cyclopropyl, fluorocyclopropyl, chlorocyclopropyl, methoxy, trifluoromethoxy, chlorodifluoromethoxy, 1,1,2,2-tetrafluoroethoxy, methylsulfanyl, propargyloxy, cyclohexyloxy, phenyloxy and pyridin-3-yloxy,
   wherein the phenyloxy and pyridin-3-yloxy is non-substituted or substituted by one or more group(s) selected from fluorine, chlorine, bromine, iodine, pentafluoro-λ⁶-sulfanyl, difluoromethyl, trifluoromethyl.
X³ more preferably represents phenyloxy or pyridin-3-yloxy, wherein the phenyloxy and pyridin-3-yloxy is substituted by one or more group(s) selected from fluorine, chlorine, bromine, iodine and trifluoromethyl.
X³ most preferably represents 4-fluorophenoxy, 4-chlorophenoxy, 4-bromophenoxy, 4-iodophenoxy, 4-(trifluoromethyl)phenoxy or pyridin-3-yloxy, wherein the pyridin-3-yloxy is substituted in 5- or 6-position by one group selected from fluorine, chlorine, bromine, iodine and trifluoromethyl.
X⁴ more preferably represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, most preferably represents hydrogen.
X⁵ more preferably represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy or methylsulfenyl, most preferably represents hydrogen, fluorine, chlorine, difluoromethyl or trifluoromethyl.
Q preferably represents a substituted 6-membered aromatic heterocycle containing one or two nitrogen atoms or a substituted 6-membered aromatic carbocycle. Substituted means that the cycle of the given formula comprises at least one of X¹, X², X³, X⁴ or X⁵ not being hydrogen.
Q more preferably represents a, preferably substituted, 6-membered aromatic cycle of formula (Q-I-1) to (Q-I-10)

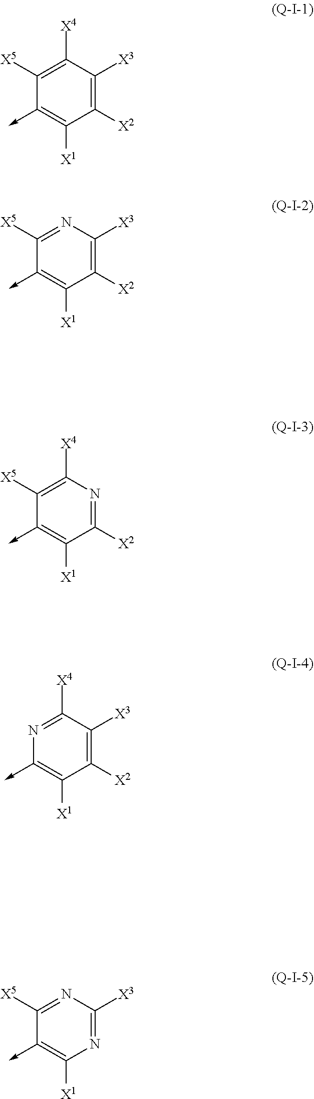

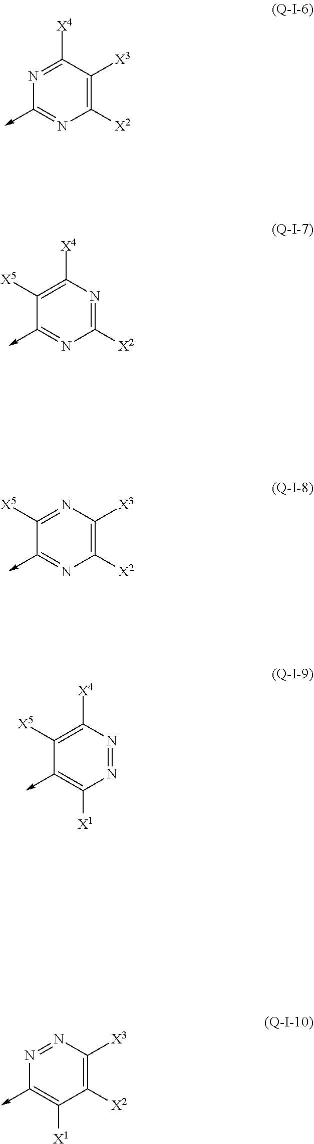

wherein X¹, X², X³, X⁴ or X⁵ have the same general, preferred, more preferred and most preferred definition as given above.
Q more preferably represents a, preferably substituted, phenyl, 3-pyridyl or 4-pyridyl of formula (Q-I-1) to (Q-I-3)

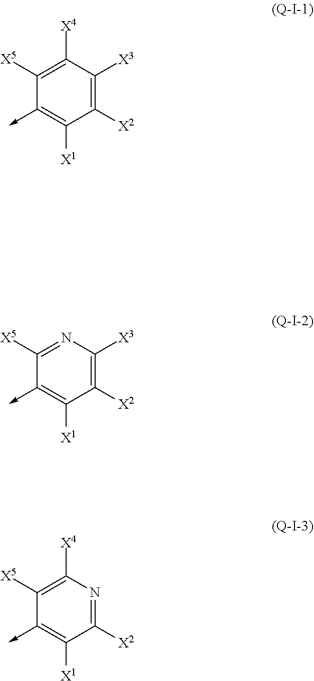

wherein X¹, X², X³, X⁴ or X⁵ have the same general, preferred, more preferred and most preferred definition as given above.
Q most preferably represents a, preferably substituted, phenyl or 3-pyridyl of formula (Q-I-1) or (Q-I-2)

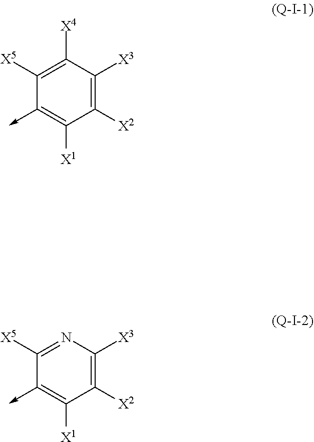

wherein X¹, X², X³, X⁴ or X⁵ have the same general, preferred, more preferred and most preferred definition as given above.

In preferred embodiments of the present invention Q represents a, preferably substituted, phenyl or 3-pyridyl of formula (Q-I-1a) or (Q-I-2a)

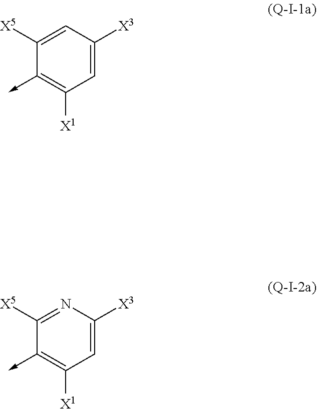

wherein X¹, X³ or X⁵ have the same general, preferred, more preferred and most preferred definition as given above.

In further preferred embodiments of the present invention the 3-pyridyl of formula (Q-I-2) is represented by formula (Q-I-2-1H)

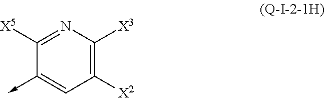

wherein
X² represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen;
X³ represents hydrogen, fluorine, pentafluoro-λ⁶-sulfanyl, methyl, ethyl, n-propyl, isopropyl, n-, iso-, sec-, tert-butyl, difluoromethyl, trifluoromethyl, cyclopropyl, fluorocyclopropyl, chlorocyclopropyl, methoxy, trifluoromethoxy, chlorodifluoromethoxy, 1,1,2,2-tetrafluoroethoxy, methylsulfanyl, propargyloxy, cyclohexyloxy, phenyloxy and pyridin-3-yloxy,
   wherein the phenyloxy and pyridin-3-yloxy is non-substituted or substituted by one or more group(s) selected from fluorine, chlorine, bromine, iodine, pentafluoro-λ⁶-sulfanyl, difluoromethyl, trifluoromethyl,
   preferably represents 4-fluorophenoxy, 4-chlorophenoxy, 4-bromophenoxy, 4-iodophenoxy, 4-(trifluoromethyl)phenoxy or pyridin-3-yloxy, wherein the pyridin-3-yloxy is substituted in 6-position by one group selected from fluorine, chlorine, bromine, iodine and trifluoromethyl; and
X⁵ represents fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably fluorine, chlorine, difluoromethyl or trifluoromethyl.

In further preferred embodiments of the present invention the 3-pyridyl of formula (Q-I-2) is represented by formula (Q-I-2-5H)

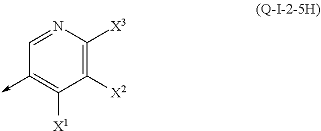

wherein
X¹ represents fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably fluorine, chlorine, difluoromethyl or trifluoromethyl;
X² represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen; and
X³ represents hydrogen, fluorine, pentafluoro-λ⁶-sulfanyl, methyl, ethyl, n-propyl, isopropyl, n-, iso-, sec-, tert-butyl, difluoromethyl, trifluoromethyl, cyclopropyl, fluorocyclopropyl, chlorocyclopropyl, methoxy, trifluoromethoxy, chlorodifluoromethoxy, 1,1,2,2-tetrafluoroethoxy, methylsulfanyl, propargyloxy, cyclohexyloxy, phenyloxy and pyridin-3-yloxy,
   wherein the phenyloxy and pyridin-3-yloxy is non-substituted or substituted by one or more group(s) selected from fluorine, chlorine, bromine, iodine, pentafluoro-λ⁶-sulfanyl, difluoromethyl, trifluoromethyl,
   preferably represents 4-fluorophenoxy, 4-chlorophenoxy, 4-bromophenoxy, 4-iodophenoxy, 4-(trifluoromethyl)phenoxy or pyridin-3-yloxy, wherein the pyridin-3-yloxy is substituted in 6-position by one group selected from fluorine, chlorine, bromine, iodine and trifluoromethyl.
R³ preferably represents halogen, hydroxyl, cyano, isocyano, nitro, carboxaldehyde, hydroxycarbonyl, C₁-C₈-cyanoalkyl, C₁-C₈-alkyloxy, C₁-C₈-haloalkyloxy, C₃-C₇-cycloalkyl, C₃-C₇-halocycloalkyl, C₂-C₈-alkenyl, C₂-C₈-alkynyl, C₂-C₈-alkenyloxy, C₂-C₈-haloalkenyloxy, C₃-C₈-alkynyloxy, C₃-C₈-haloalkynyloxy, C₁-C₈-alkylsulfanyl, C₁-C₈-haloalkylsulfanyl, C₁-C₈-alkylcarbonyl, C₁-C₈-haloalkylcarbonyl, arylcarbonyl, aryl-C₁-C₆-alkylcarbonyl, C₃-C₈-cycloalkylcarbonyl, C₃-C₈-halocycloalkylcarbonyl, aminothiocarbonyl, C₁-C₈-alkoxycarbonyl, C₁-C₈-haloalkoxycarbonyl, C₃-C₈-cycloalkoxycarbonyl, C₁-C₈-alkylcarbonyloxy, C₁-C₈-haloalkylcarbonyloxy, C₃-C₈-cycloalkylcarbonyloxy, benzyl, phenyl, 5-membered heteroaryl, 6-membered heteroaryl, benzyloxy, or phenyloxy, wherein the benzyl, phenyl, 5-membered heteroaryl, 6-membered heteroaryl, benzyloxy or phenyloxy may be optionally substituted by one or more group(s) selected from halogen, hydroxyl, cyano, isocyano, amino, sulfanyl, pentafluoro-λ⁶-sulfanyl, C₁-C₈-alkyl, C₁-C₈-haloalkyl, C₁-C₈-alkyloxy, C₁-C₈-haloalkyloxy, tri(C₁-C₈-alkyl)silyl, C₃-C₇-cycloalkyl, C₂-C₈-alkenyl, C₂-C₈-alkynyl.
R³ more preferably represents halogen, cyano, carboxaldehyde, hydroxycarbonyl, C₂-C₈-alkyl, C₁-C₈-haloalkyl, C₁-C₈-cyanoalkyl, C₁-C₈-alkyloxy, C₁-C₈-haloalkyloxy, C₃-C₇-cycloalkyl, C₃-C₇-halocycloalkyl, C₂-C₈-alkenyl, C₂-C₈-alkynyl, C₁-C₈-alkylsulfanyl, C₁-C₈-haloalkylsulfanyl, C₁-C₈-alkylcarbonyl, C₁-C₈-haloalkylcarbonyl, aminothiocarbonyl, C₁-C₈-alkoxycarbonyl, C₁-C₈-haloalkoxycarbonyl, benzyl, phenyl, 5-membered heteroaryl, 6-membered heteroaryl, benzyloxy, or phenyloxy, wherein the benzyl, phenyl, 5-membered heteroaryl, 6-membered heteroaryl, benzyloxy or phenyloxy may be optionally substituted by one or more group(s) selected from halogen, hydroxyl, cyano, amino, sulfanyl, pentafluoro-λ⁶-sulfanyl, C₁-C₈-alkyl, C₁-C₈-haloalkyl, C₁-C₈-alkyloxy, C₁-C₈-haloalkyloxy, C₃-C₇-cycloalkyl, C₂-C₈-alkenyl, C₂-C₈-alkynyl.
R³ more preferably represents halogen, cyano, carboxaldehyde, hydroxycarbonyl, C₂-C₄-alkyl, C₁-C₄-haloalkyl, C₁-C₄-cyanoalkyl, C₁-C₄-alkyloxy, C₁-C₄-haloalkyloxy, C₃-C₇-cycloalkyl, C₃-C₇-halocycloalkyl, C₂-C₅-alkenyl, C₂-C₅-alkynyl, C₁-C₄-alkylsulfanyl, C₁-C₄-haloalkylsulfanyl, C₁-C₄-alkylcarbonyl, C₁-C₄-haloalkylcarbonyl, aminothiocarbonyl, C₁-C₄-alkoxycarbonyl, C₁-C₄-haloalkoxycarbonyl, benzyl, phenyl, furyl, pyrrolyl, thienyl, pyridyl, benzyloxy, or phenyloxy, wherein the benzyl, phenyl, 5-membered heteroaryl, 6-membered heteroaryl, benzyloxy or phenyloxy may be optionally substituted by one or more group(s) selected from halogen, C₁-C₈-alkyl, C₁-C₈-haloalkyl, C₁-C₈-alkyloxy, C₁-C₈-haloalkyloxy.
R³ more preferably represents fluorine, chlorine, bromine, iodine, cyano, hydroxycarbonyl, carboxaldehyde, C₂-C₄-alkyl, C₁-C₄-haloalkyl, C₁-C₄-cyanoalkyl, C₁-C₄-alkyloxy, C₃-C₇-cycloalkyl, C₂-C₅-alkynyl, C₁-C₄-alkylsulfanyl, C₁-C₄-alkylcarbonyl, aminothiocarbonyl, C₁-C₄-alkoxycarbonyl, phenyl, or thienyl, wherein the phenyl or thienyl may be optionally substituted by one or more group(s) selected from halogen, C₁-C₈-alkyl, C₁-C₈-haloalkyl, C₁-C₈-alkyloxy, C₁-C₈-haloalkyloxy.
R³ more preferably represents fluorine, chlorine, bromine, iodine, cyano, hydroxycarbonyl, carboxaldehyde, trifluoromethyl, cyanomethyl, methoxy, methylsulfanyl, cyclopropyl, ethinyl, methylcarbonyl (acetyl), carboxyl, aminothiocarbonyl, methoxycarbonyl, ethoxycarbonyl, phenyl, or 2-thienyl.
R³ more preferably represents fluorine, chlorine, bromine, cyano, trifluoromethyl, or ethinyl.
R³ more preferably represents fluorine, chlorine, bromine, iodine, or cyano.
R³ more preferably represents chlorine, fluorine or cyano.
R³ most preferably represents cyano.

In one preferred embodiment of the invention R² represents naphthyl, thiazolyl or thienyl, preferably naphthyl, 1,3-thiazol-5-yl, 1,3-thiazol-4-yl, 2-thienyl or 3-thienyl, wherein

the naphthyl, thiazolyl, thienyl, 1,3-thiazol-5-yl, 1,3-thiazol-4-yl, 2-thienyl, 3-thienyl is non-substituted or substituted by one or more group(s) selected from halogen, cyano, sulfanyl, pentafluoro-λ⁶-sulfanyl, C₁-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-haloalkenyl, C₂-C₈-alkynyl, C₂-C₈-haloalkynyl, C₂-C₈-alkenyloxy, C₂-C₈-haloalkenyloxy, C₃-C₈-alkynyloxy, C₃-C₈-haloalkynyloxy, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, 6-membered heteroaryloxy, benzyloxy, phenoxy, 4-halogen-substituted phenoxy, 4-(C₁-C₈-haloalkyl)-substituted phenoxy, benzylsulfanyl or phenylsulfanyl; preferably is non-substituted or substituted by one or more group(s) selected from halogen, cyano, pentafluoro-λ⁶-sulfanyl, C₁-C₈-alkyl, C₁-C₈-haloalkyl, C₁-C₈-alkyloxy, C₁-C₈-haloalkyloxy, tri(C₁-C₈-alkyl)silyl, C₃-C₇-cycloalkyl, C₃-C₇-halocycloalkyl, C₂-C₈-alkenyl, C₂-C₈-alkynyl, C₁-C₈-alkylsulfanyl, C₁-C₈-haloalkylsulfanyl, benzyl, phenyl, 5-membered heteroaryl, 6-membered heteroaryl, 6-membered heteroaryloxy, benzyloxy, phenoxy, 4-halogen-substituted phenoxy, 4-(C₁-C₈-haloalkyl)-substituted phenoxy, benzylsulfanyl, or phenylsulfanyl, more preferably is non-substituted or substituted by one or more group(s) selected from halogen, pentafluoro-λ⁶-sulfanyl, C₁-C₈-alkyl, C₁-C₈-haloalkyl, C₁-C₈-alkyloxy, C₁-C₈-haloalkyloxy, C₁-C₈-haloalkylsulfanyl, phenyl, 5-membered heteroaryl, 6-membered heteroaryl, 6-membered heteroaryloxy, phenoxy, 4-halogen-substituted phenoxy, 4-(C₁-C₈-haloalkyl)-substituted phenoxy, or phenylsulfanyl, more preferably is non-substituted or substituted by one or more group(s) selected from fluorine, chlorine, bromine, iodine, pentafluoro-λ⁶-sulfanyl, methyl, ethyl, n-propyl, isopropyl, n-, iso-, sec-, tert-butyl, difluoromethyl, trifluoromethyl, methoxy, trifluoromethoxy, chlorodifluoromethoxy, 1,1,2,2-tetrafluoroethoxy, methylsulfanyl, trifluoromethylsulfanyl, phenyl, pyridinyloxy, phenoxy, 4-fluorophenoxy, 4-chlorophenoxy, 4-bromophenoxy, 4-iodophenoxy, 4-(trifluoromethyl)-substituted phenoxy, or phenylsulfanyl, most preferably is substituted by one or more, preferably by one group(s) selected from 4-fluorophenoxy, 4-chlorophenoxy, 4-bromophenoxy, 4-iodophenoxy, and 4-(trifluoromethyl)-substituted phenoxy.

In another preferred embodiment of the invention R² represents a substituent of formula Q, wherein Q is defined in general, preferred, more preferred and most preferred terms as outlined above.

Preferred embodiments according to the present invention are compounds of formula (I), wherein
R³ represents halogen or cyano; and

A, R¹, R^{a}, R^{b}, R², Q, U¹, U², U³, U⁴, U⁵, X¹, X², X³, X⁴, and X⁵ have the same general, preferred, more preferred and most preferred definition as given for formula (I).

In such preferred embodiments according to the present invention R³ preferably represents fluorine; chlorine; bromine; iodine or cyano, more preferably cyano, and A, R¹, R^{a}, R^{b}, R², Q, U¹, U², U³, U⁴, U⁵, X¹, X², X³, X⁴, and X⁵ have the same general, preferred, more preferred and most preferred definition as given for formula (I).

A preferred embodiment of the present invention relates to compounds of formula (I-1)

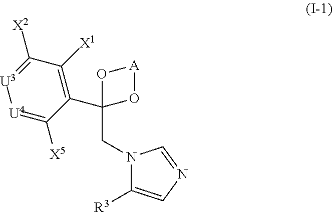

wherein A, R³, U³, U⁴, X¹, X² and X⁵ have the same general, preferred, more preferred and most preferred definition as given for formula (I).

A further preferred embodiment of the present invention relates to compounds of formula (I-1-Q-I-1)

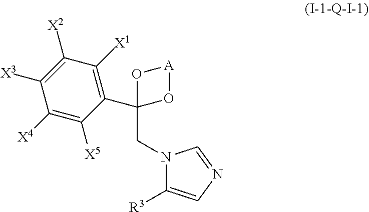

wherein A, R³, X¹, X², X³, X⁴ and X⁵ have the same general, preferred, more preferred and most preferred definition as given for formula (I).

Particularly preferred are compounds of formula (I-1-Q-I-1)

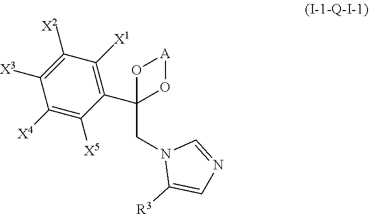

wherein
A represents ethylene, 1,2-propylene, 1,2-butylene, 2,3-butylene or 1,2-pentylene, preferably ethylene or 1,2-propylene;
R³ represents fluorine, chlorine, bromine, iodine, cyano, hydroxycarbonyl, carboxaldehyde, trifluoromethyl, cyanomethyl, methoxy, methylsulfanyl, cyclopropyl, ethinyl, methylcarbonyl (acetyl), carboxyl, aminothiocarbonyl, methoxycarbonyl, ethoxycarbonyl, phenyl, or 2-thienyl, preferably fluorine, chlorine, bromine, cyano, or trifluoromethyl;
X¹ represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen;
X² represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen;
X³ represents phenyloxy or pyridin-3-yloxy, wherein the phenyloxy and pyridin-3-yloxy is substituted by one or more group(s) selected from fluorine, chlorine, bromine, iodine and trifluoromethyl; preferably represents 4-fluorophenoxy, 4-chlorophenoxy, 4-bromophenoxy, 4-iodophenoxy, 4-(trifluoromethyl)phenoxy or pyridin-3-yloxy, wherein the pyridin-3-yloxy is substituted in 5- or 6-position by one group selected from fluorine, chlorine, bromine, iodine and trifluoromethyl;
X⁴ represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen; and
X⁵ represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen, fluorine, chlorine, difluoromethyl or trifluoromethyl.

A further preferred embodiment of the present invention relates to compounds of the formula (I-1-Q-I-2)

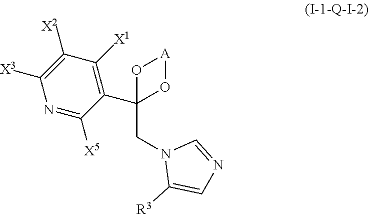

wherein A, R³, X¹, X², X³ and X⁵ have the same general, preferred, more preferred and most preferred definition as given for formula (I).

Particularly preferred are compounds of formula (I-1-Q-I-2)

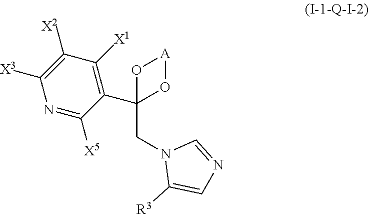

wherein
A represents ethylene, 1,2-propylene, 1,2-butylene, 2,3-butylene or 1,2-pentylene, preferably ethylene or 1,2-propylene;
R³ represents fluorine, chlorine, bromine, iodine, cyano, hydroxycarbonyl, carboxaldehyde, trifluoromethyl, cyanomethyl, methoxy, methylsulfanyl, cyclopropyl, ethinyl, methylcarbonyl (acetyl), carboxyl, aminothiocarbonyl, methoxycarbonyl, ethoxycarbonyl, phenyl, or 2-thienyl, preferably fluorine, chlorine, bromine, cyano, or trifluoromethyl;
X¹ represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy or methylsulfenyl, preferably hydrogen, fluorine, chlorine, difluoromethyl or trifluoromethyl;
X² represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen;
X³ represents phenyloxy or pyridin-3-yloxy, wherein the phenyloxy and pyridin-3-yloxy is substituted by one or more group(s) selected from fluorine, chlorine, bromine, iodine and trifluoromethyl; preferably represents 4-fluorophenoxy, 4-chlorophenoxy, 4-bromophenoxy, 4-iodophenoxy, 4-(trifluoromethyl)phenoxy or pyridin-3-yloxy, wherein the pyridin-3-yloxy is substituted in 5- or 6-position by one group selected from fluorine, chlorine, bromine, iodine and trifluoromethyl; and
X⁵ represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen, fluorine, chlorine, difluoromethyl or trifluoromethyl.

A further preferred embodiment of the present invention relates to compounds of formula (I-1-Q-I-3)

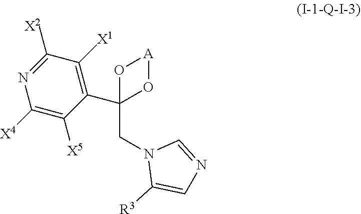

wherein A, R³, X¹, X², X⁴ and X⁵ have the same general, preferred, more preferred and most preferred definition as given for formula (I).

Particularly preferred are compounds of formula (I-1-Q-I-3)

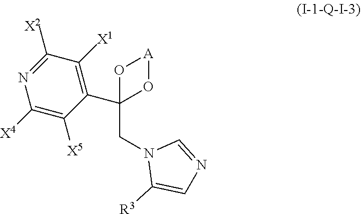

wherein
A represents ethylene, 1,2-propylene, 1,2-butylene, 2,3-butylene or 1,2-pentylene, preferably ethylene or 1,2-propylene;
R³ represents fluorine, chlorine, bromine, iodine, cyano, hydroxycarbonyl, carboxaldehyde, trifluoromethyl, cyanomethyl, methoxy, methylsulfanyl, cyclopropyl, ethinyl, methylcarbonyl (acetyl), carboxyl, aminothiocarbonyl, methoxycarbonyl, ethoxycarbonyl, phenyl, or 2-thienyl, preferably fluorine, chlorine, bromine, cyano, or trifluoromethyl;
X¹ represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen;
X² represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen;
X⁴ represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen; and
X⁵ represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, preferably hydrogen, fluorine, chlorine, difluoromethyl or trifluoromethyl.

The radical definitions and explanations given above in general terms or stated within preferred ranges can, however, also be combined with one another as desired, i.e. including between the particular ranges and preferred ranges. They apply both to the end products and correspondingly to precursors and intermediates. In addition, individual definitions may not apply.

Preference is given to those compounds of the formula (I) in which each of the radicals have the abovementioned preferred definitions.

Particular preference is given to those compounds of the formula (I) in which each of the radicals have the abovementioned more preferred definitions.

Very particular preference is given to those compounds of the formula (I) in which each of the radicals have the above mentioned most preferred definitions.

In the definitions of the symbols given in the above formulae, collective terms were used which are generally representative of the following substituents:

The definition C₁-C₈-alkyl comprises the largest range defined here for an alkyl radical. Specifically, this definition comprises the meanings methyl, ethyl, n-, isopropyl, n-, iso-, sec-, tert-butyl, and also in each case all isomeric pentyls, hexyls, heptyls and octyls, such as methyl, ethyl, propyl, 1-methylethyl, butyl, 1-methylpropyl, 2-methylpropyl, 1,1-dimethylethyl, n-pentyl, 1-methylbutyl, 2-methylbutyl, 3-methylbutyl, 1,2-dimethylpropyl, 1,1-dimethylpropyl, 2,2-dimethylpropyl, 1-ethylpropyl, n-hexyl, 1-methylpentyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-3-methylpropyl, n-heptyl, 1-methylhexyl, 1-ethylpentyl, 2-ethylpentyl, 1-propylbutyl, octyl, 1-methylheptyl, 2-methylheptyl, 1-ethylhexyl, 2-ethylhexyl, 1-propylpentyl and 2-propylpentyl, in particular propyl, 1-methylethyl, butyl, 1-methylbutyl, 2-methylbutyl, 3-methylbutyl, 1,1-dimethylethyl, 1,2-dimethylbutyl, 1,3-dimethylbutyl, pentyl, 1-methylbutyl, 1-ethylpropyl, hexyl, 3-methylpentyl, heptyl, 1-methylhexyl, 1-ethyl-3-methylbutyl, 1-methylheptyl, 1,2-dimethylhexyl, 1,3-dimethyloctyl, 4-methyloctyl, 1,2,2,3-tetramethylbutyl, 1,3,3-trimethylbutyl, 1,2,3-trimethylbutyl, 1,3-dimethylpentyl, 1,3-dimethylhexyl, and 5-methyl-3-hexyl, 2-methyl-4-heptyl. A preferred range is C₁-C₄-alkyl, such as methyl, ethyl, n-, isopropyl, n-, iso-, sec-, tert-butyl. The definition C₁-C₃-alkyl comprises methyl, ethyl, n-, isopropyl.

The definition halogen comprises fluorine, chlorine, bromine and iodine. Halogen-substitution is generally indicated by the prefix halo, halogen or halogeno.

Halogen-substituted alkyl—referred to as haloalkyl, halogenalkyl or halogenoalkyl—represents, for example, C₁-C₈-alkyl as defined above substituted by one or more halogen substituents which can be the same or different. Preferably C₁-C₈-haloalkyl represents chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1-fluoro-1-methylethyl, 2-fluoro-1,1-dimethylethyl, 2-fluoro-1-fluoromethyl-1-methylethyl, 2-fluoro-1,1-di(fluoromethyl)-ethyl, 3-chloro-1-methylbutyl, 2-chloro-1-methylbutyl, 1-chlorobutyl, 3,3-dichloro-1-methylbutyl, 3-chloro-1-methylbutyl, 1-methyl-3-trifluoromethylbutyl, 3-methyl-1-trifluoromethylbutyl.

Mono- or multiple fluorinated C₁-C₄-alkyl represents, for example, C₁-C₄-alkyl as defined above substituted by one or more fluorine substituent(s). Preferably mono- or multiple fluorinated C₁-C₄-alkyl represents fluoromethyl, difluoromethyl, trifluoromethyl, 1-fluoroethyl, 2-fluoroethyl, 2,2-difluoroethyl, 2,2,2-trifluoroethyl, pentafluoroethyl, 1-fluoro-1-methylethyl, 2-fluoro-1,1-dimethylethyl, 2-fluoro-1-fluoromethyl-1-methylethyl, 2-fluoro-1,1-di(fluoromethyl)-ethyl, 1-methyl-3-trifluoromethylbutyl, 3-methyl-1-trifluoromethylbutyl.

The definition C₂-C₈-alkenyl comprises the largest range defined here for an alkenyl radical. Specifically, this definition comprises the meanings ethenyl, n-, isopropenyl, n-, iso-, sec-, tert-butenyl, and also in each case all isomeric pentenyls, hexenyls, heptenyls, octenyls, 1-methyl-1-propenyl, 1-ethyl-1-butenyl, 2,4-dimethyl-1-pentenyl, 2,4-dimethyl-2-pentenyl. Halogen-substituted alkenyl—referred to as haloalkenyl, halogenalkenyl or halogenoalkenyl—represents, for example, C₂-C₈-alkenyl as defined above substituted by one or more halogen substituents which can be the same or different. A preferred range is C₂-C₄-alkenyl, such as ethenyl, n-, isopropenyl, n-, iso-, sec- or tert-butenyl.

The definition C₂-C₈-alkynyl comprises the largest range defined here for an alkynyl radical. Specifically, this definition comprises the meanings ethynyl, n-, isopropynyl, n-, iso-, sec-, tert-butynyl, and also in each case all isomeric pentynyls, hexynyls, heptynyls, octynyls. Halogen-substituted alkynyl—referred to as haloalkynyl, halogenalkynyl or halogenoalkynyl—represents, for example, C₂-C₈-alkynyl as defined above substituted by one or more halogen substituents which can be the same or different. A preferred range is C₂-C₄-alkynyl, such as ethynyl, n-, isopropynyl, n-, iso-, sec- or tert-butynyl.

The definition C₃-C₇-cycloalkyl comprises monocyclic saturated hydrocarbyl groups having 3 to 7 carbon ring members, such as cyclopropyl, cyclobutyl, cyclopentyl, cyclohexyl and cycloheptyl.

The definition halogen-substituted cycloalkyl and halocycloalkyl comprises monocyclic saturated hydrocarbyl groups having 3 to 7 carbon ring members, such as 1-fluoro-cyclopropyl and 1-chloro-cyclopropyl.

The definition bicycloalkyl comprises spirocyclic alkyl wherein two substituents at the same carbon atom of a C₃-C₇-cycloalkyl can form together with the carbon atom to which they are attached a C₃-C₇-cycloalkyl, this definition comprises for example the meaning spiro[2.2]pentyl. The definition bicycloalkyl also comprises bicyclic alkyls wherein two substituents at different adjacent or non-adjacent carbon atoms of a C₃-C₇-cycloalkyl can form together with the carbon atoms to which they are attached a C₃-C₇-cycloalkyl, this definition comprises for example the meaning bicyclo[2.2.1]heptane-2-yl, bicyclo[2.2.1]heptane-7-yl, bicyclo[4.1.0]heptane-2-yl, bicyclo[4.1.0]heptane-3-yl, bicyclo[4.1.0]heptane-7-yl. The definition bicycloalkyl also comprises bicyclic alkyls wherein two substituents at different adjacent or non-adjacent carbon atoms of a C₃-C₇-cycloalkyl can form an alkylene bridge between the carbon atoms to which they are attached, this definition comprises for example the meaning bicyclo[2.2.1]hept-2-ene-2-yl, bicyclo[2.2.1]hept-2-ene-5-yl, bicyclo[2.2.1]hept-2-ene-7-yl.

The definition aryl comprises aromatic, mono-, bi- or tricyclic carbocycles, for example phenyl, naphthyl, anthracenyl (anthryl), phenanthracenyl (phenanthryl).

The definition hetaryl or heteroaryl comprises unsaturated, benzoannulated or not benzoannulated heterocyclic 5- to 10-membered ring containing up to 4 heteroatoms selected from N, O and S. Preferably The definition hetaryl or heteroaryl comprises unsubstituted or substituted, unsaturated heterocyclic 5- to 7-membered ring containing up to 4 heteroatoms selected from N, O and S: for example 2-furyl, 3-furyl, 2-thienyl, 3-thienyl, 2-pyrrolyl, 3-pyrrolyl, 1-pyrrolyl, 3-pyrazolyl, 4-pyrazolyl, 5-pyrazolyl, 1-pyrazolyl, 1H-imidazol-2-yl, 1H-imidazol-4-yl, 1H-imidazol-5-yl, 1H-imidazol-1-yl, 2-oxazolyl, 4-oxazolyl, 5-oxazolyl, 2-thiazolyl, 4-thiazolyl, 5-thiazolyl, 3-isoxazolyl, 4-isoxazolyl, 5-isoxazolyl, 3-isothiazolyl, 4-isothiazolyl, 5-isothiazolyl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-3-yl, 1H-1,2,4-triazol-5-yl, 1H-1,2,4-triazol-1-yl, 4H-1,2,4-triazol-3-yl, 4H-1,2,4-triazol-4-yl, 1H-tetrazol-1-yl, 1H-tetrazol-5-yl, 2H-tetrazol-2-yl, 2H-tetrazol-5-yl, 1,2,4-oxadiazol-3-yl, 1,2,4-oxadiazol-5-yl, 1,2,4-thiadiazol-3-yl, 1,2,4-thiadiazol-5-yl, 1,3,4-oxadiazol-2-yl, 1,3,4-thiadiazol-2-yl, 1,2,3-oxadiazol-4-yl, 1,2,3-oxadiazol-5-yl, 1,2,3-thiadiazol-4-yl, 1,2,3-thiadiazol-5-yl, 1,2,5-oxadiazol-3-yl, 1,2,5-thiadiazol-3-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,4-triazin-6-yl.

The definition 5-membered heteroaryl comprises an unsaturated heterocyclic 5-membered ring containing up to 4 heteroatoms selected from N, O and S: for example 2-furyl, 3-furyl, 2-thienyl, 3-thienyl, 2-pyrrolyl, 3-pyrrolyl, 1-pyrrolyl, 3-pyrazolyl, 4-pyrazolyl, 5-pyrazolyl, 1-pyrazolyl, 1H-imidazol-2-yl, 1H-imidazol-4-yl, 1H-imidazol-5-yl, 1H-imidazol-1-yl, 2-oxazolyl, 4-oxazolyl, 5-oxazolyl, 2-thiazolyl, 4-thiazolyl, 5-thiazolyl, 3-isoxazolyl, 4-isoxazolyl, 5-isoxazolyl, 3-isothiazolyl, 4-isothiazolyl, 5-isothiazolyl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-3-yl, 1H-1,2,4-triazol-5-yl, 1H-1,2,4-triazol-1-yl, 4H-1,2,4-triazol-3-yl, 4H-1,2,4-triazol-4-yl, 1H-tetrazol-1-yl, 1H-tetrazol-5-yl, 2H-tetrazol-2-yl, 2H-tetrazol-5-yl, 1,2,4-oxadiazol-3-yl, 1,2,4-oxadiazol-5-yl, 1,2,4-thiadiazol-3-yl, 1,2,4-thiadiazol-5-yl, 1,3,4-oxadiazol-2-yl, 1,3,4-thiadiazol-2-yl, 1,2,3-oxadiazol-4-yl, 1,2,3-oxadiazol-5-yl, 1,2,3-thiadiazol-4-yl, 1,2,3-thiadiazol-5-yl, 1,2,5-oxadiazol-3-yl, 1,2,5-thiadiazol-3-yl.

The definition 6-membered heteroaryl comprises an unsaturated heterocyclic 6-membered ring containing up to 4 heteroatoms selected from N, O and S: for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,4-triazin-6-yl.

The definition heterocycloalkyl comprises saturated or partially unsaturated mono-, bi- or tricyclic ring systems consisting of C-atoms and containing up to 4 heteroatoms selected from N, O and S: for example aziridinyl, pyrrolidinyl, dihydropyridyl, piperidinyl, piperazinyl, morpholinyl, thiomorpholinyl, tetrahydrofuranyl, tetrahydrothiofuranyl, tetrahydropyranyl, pyranyl, isoxazolidinyl, isoxazolinyl, pyrazolinyl, dihydropyrrolyl, tetrahydropyridinyl, dioxolanyl, dioxanyl, oxathiolanyl, oxathianyl, dithiolanyl, dithianyl. The term partially unsaturated refers to ring systems that are neither saturated, i.e. comprising no double bound, nor fully unsaturated, i.e. comprising the maximum possible number of double bonds. In other words, partially unsaturated ring systems comprise at least one double bond, but not the maximum possible number of double bonds.

Optionally substituted radicals may be mono- or polysubstituted, where in the case of polysubstitution, the substituents may be identical or different.

Unless indicated otherwise, a group or a substituent which is substituted according to the invention preferably can be substituted by one or more group(s) selected from the list consisting of halogen, SH, nitro, hydroxyl, cyano, amino, sulfanyl, pentafluoro-λ⁶-sulfanyl, formyl, formyloxy, formylamino, carbamoyl, N-hydroxycarbamoyl, carbamate, (hydroxyimino)-C₁-C₆-alkyl, C₁-C₈-halogenalkyl, C₁-C₈-alkyloxy, C₁-C₈-halogenalkyloxy, C₁-C₈-alkylthio, C₁-C₈-halogenalkylthio, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, tri(C₁-C₈-alkyl)silyl-C₃-C₇-cycloalkyl, C₁-C₈-halogenoalkyl, C₃-C₇-halogenocycloalkyl, C₂-C₈-alkenyl, C₂-C₈-alkynyl, C₂-C₈-alkenyloxy, C₂-C₈-halogenalkenyloxy, C₂-C₈-alkynyloxy, C₁-C₈-alkylamino, di-C₁-C₈-alkylamino, C₁-C₈-halogenalkylamino, di-C₁-C₈-halogenalkylamino, C₁-C₈-alkylaminoalkyl, di-C₁-C₈-alkylaminoalkyl, C₁-C₈-alkoxy, C₁-C₈-halogenoalkoxy, C₁-C₈-cyanoalkoxy, C₄-C₈-cycloalkylalkoxy, C₃-C₆-cycloalkoxy, C₂-C₈-alkoxyalkoxy, C₁-C₈-alkylcarbonylalkoxy, C₁-C₈-alkylsulfanyl, C₁-C₈-halogenoalkylsulfanyl, C₂-C₈-alkenyloxy, C₂-C₈-halogenoalkenyloxy, C₃-C₈-alkynyloxy, C₃-C₈-halogenoalkynyloxy, C₁-C₈-alkylcarbonyl, C₁-C₈-halogenoalkylcarbonyl, C₃-C₈-cycloalkylcarbonyl, C₃-C₈-halogenocycloalkylcarbonyl, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl, C₃-C₈-cycloalkoxycarbonyl, C₂-C₈-alkoxyalkylcarbonyl, C₂-C₈-halogenoalkoxyalkylcarbonyl, C₃-C₁₀-cycloalkoxyalkylcarbonyl, C₁-C₈-alkylaminocarbonyl, di-C₁-C₈-alkylaminocarbonyl, C₃-C₈-cycloalkylaminocarbonyl, C₁-C₈-alkylcarbonyloxy, C₁-C₈-halogenoalkylcarbonyloxy, C₃-C₈-cycloalkylcarbonyloxy, C₁-C₈-alkylcarbonylamino, C₁-C₈-halogenoalkylcarbonylamino, C₁-C₈-alkylaminocarbonyloxy, di-C₁-C₈-alkylaminocarbonyloxy, C₁-C₈-alkyloxycarbonyloxy, C₁-C₈-alkylsulfinyl, C₁-C₈-halogenoalkylsulfinyl, C₁-C₈-alkylsulfonyl, C₁-C₈-halogenoalkylsulfonyl, C₁-C₈-alkylsulfonyloxy, C₁-C₈-halogenoalkylsulfonyloxy, C₁-C₈-alkylaminosulfamoyl, di-C₁-C₈-alkylaminosulfamoyl, (C₁-C₈-alkoxyimino)-C₁-C₈-alkyl, (C₃-C₇-cycloalkoxyimino)-C₁-C₈-alkyl, hydroxyimino-C₁-C₈-alkyl, (C₁-C₈-alkoxyimino)-C₃-C₇-cycloalkyl, hydroxyimino-C₃-C₇-cycloalkyl, (C₁-C₈-alkylimino)-oxy, (C₁-C₈-alkylimino)-oxy-C₁-C₈-alkyl, (C₃-C₇-cycloalkylimino)-oxy-C₁-C₈-alkyl, (C₁-C₆-alkylimino)-oxy-C₃-C₇-cycloalkyl, (C₁-C₈-alkenyloxyimino)-C₁-C₈-alkyl, (C₁-C₈-alkynyloxyimino)-C₁-C₈-alkyl, (benzyloxyimino)-C₁-C₈-alkyl, C₁-C₈-alkoxyalkyl, C₁-C₈-alkylthioalkyl, C₁-C₈-alkoxyalkoxyalkyl, C₁-C₈-halogenoalkoxyalkyl, benzyl, phenyl, 5-membered heteroaryl, 6-membered heteroaryl, benzyloxy, phenyloxy, benzylsulfanyl, benzylamino, phenoxy, phenylsulfanyl, or phenylamino, wherein the benzyl, phenyl, 5-membered heteroaryl, 6-membered heteroaryl, benzyloxy or phenyloxy may be optionally substituted by one or more group(s) selected from the aforementioned list.

Depending on the nature of the substituents, the compounds according to the invention can be present as mixtures of different possible isomeric forms, in particular of stereoisomers, such as, for example, E and Z, threo and eiythro, and also optical isomers, and, if appropriate, also of tautomers. What is claimed are both the E and the Z isomers, and also the threo and eiythro, and the optical isomers, any mixtures of these isomers, and the possible tautomeric forms.

Depending on the nature of the substituents, the compounds of the present invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions) and to the mixtures of all the possible stereoisomers, in all proportions. The diastereoisomers and/or the optical isomers can be separated according to the methods which are known per se by the man ordinary skilled in the art.

Depending on the nature of the substituents, the compounds of the present invention can also exist in one or more geometric isomer forms depending on the number of double bonds in the compound. The invention thus relates equally to all geometric isomers and to all possible mixtures, in all proportions. The geometric isomers can be separated according to general methods, which are known per se by the man ordinary skilled in the art.

Depending on the nature of the substituents, the compounds of the present invention can also exist in one or more geometric isomer forms depending on the relative position (syn/anti or cis/trans) of the substituents of a ring. The invention thus relates equally to all syn/anti (or cis/trans) isomers and to all possible syn/anti (or cis/trans) mixtures, in all proportions. The syn/anti (or cis/trans) isomers can be separated according to general methods, which are known per se by the man ordinary skilled in the art.

The compounds of formula (I) wherein Q is substituted by a hydroxy, a sulfanyl or an amino substituent may be found in its tautomeric form resulting from the shift of the proton of said hydroxy, sulfanyl or amino group. All tautomeric forms of such compounds of the present invention) wherein Q is substituted by a hydroxy, a sulfanyl or an amino substituent are also part of the present invention.

Illustration of the Processes and Intermediates

The present invention furthermore relates to processes for preparing compounds of formula (I). The present invention furthermore relates to intermediates and the preparation thereof.

The compounds (I) can be obtained by various routes in analogy to prior art processes known (see e.g. WO-A 2010/146114; J. Agric. Food Chem. (2009) 57, 4854-4860; EP-A 0 275 955; DE-A 40 03 180; WO-A 2010/146116; WO-A 2013/007767 and references cited therein) and by synthesis routes shown schematically below and in the experimental part of this application. Unless indicated otherwise, the radicals A, Q, R¹, and R³ have the meanings given above for the compounds of formula (I). These definitions apply not only to the end products of the formula (I) but likewise to all intermediates.

Process A (Scheme 1):

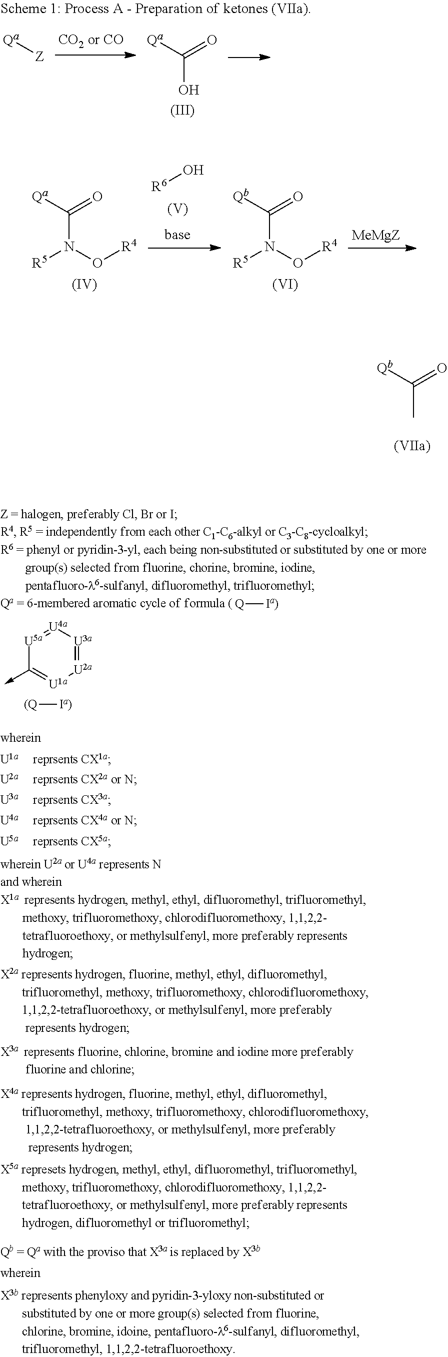

Compounds of formula (II) (Scheme 1) can be converted by means of methods described in the literature to the corresponding compounds (III). Compounds (II), wherein Z stands for halogen, preferably Cl, Br or I, are optionally reacted with carbon dioxide or formate salts to obtain compounds (III). This transformation is performed in the presence of reagents or catalysts such as lithium, magnesium, n-butyllithium, methyllithium or nickel (e.g. Organic & Biomolecular Chemistry, 8(7), 1688-1694; 2010; WO-A 2003/033504; Organometallics, 13(11), 4645-7; 1994 and references cited therein). Alternatively, compound (II) is reacted in a hydroxycarbonylation reaction with carbon monoxide or a formate salt, preferentially in the presence of a catalyst such as Pd(OAc)₂ and Co(OAc)₂ (e.g. Dalton Transactions, 40(29), 7632-7638; 2011; Synlett, (11), 1663-1666; 2006 and references cited therein).

Weinreb amides of formula (IV) can be obtained by reaction of acid (III) with chlorinating agents such as thionyl chloride or oxalyl chloride, followed by treatment with alkoxyalkylamine, preferentially methoxymethylamine. Alternatively, the conversion of acid (III) to Weinreb amide (IV) can be carried out in the presence of reagents such as carbodiimides (e.g. WO-A 2011/076744), diimidazolyl ketone CDI, N-alkoxy-N-alkylcarbamoyl chlorides (e.g. Bulletin of the Korean Chemical Society 2002, 23, 521-524), S,S-di-2-pyridyl dithiocarbonates (e.g. Bulletin of the Korean Chemical Society 2001, 22, 421-423), trichloromethyl chloroformate (e.g. Synthetic communications 2003, 33, 4013-4018) or peptide coupling reagent HATU. Compounds of formula (VI) are obtained by reaction of Weinreb amide (IV) and alcohols of formula (V), optionally in the presence of a base such as K₂CO₃, Cs₂CO₃, NEt₃, K₃PO₄ or DABCO and a solvent such as DMF or DMSO. Those reactions may be performed in the presence of a metal catalyst such as CuI in the presence of TMEDA. Ketones of formula (VIIa) can be obtained by reaction of compounds (VI) with magnesium halides MeMgQ such as methylmagnesium bromide or methylmagnesium chloride, preferentially in a solvent such as THF.

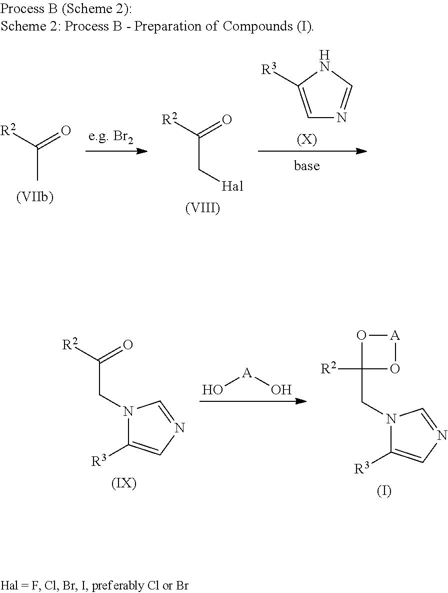

Ketones of formula (VIIb) are commercially available or can be made by means of methods described in the literature (e.g. WO-A 2010/146114, J. Agric. Food Chem. (2009) 57, 4854-4860). In case R² is represented by Q^{b}, they can also be made according to the method described in Scheme 1.

Ketones of formula (VIIb) can then be halogenated, for instance with Cl₂, Br₂, ammonium dichloroiodates, such as for instance benzyltrimethylammonium dichloroiodate, or ammonium tribromides, such as tetra-n-butylammonium tribromide, in order to obtain α-haloketones (VIII). The reactions are preferably carried out in an organic solvent such as diethyl ether, methyl tert-butyl ether, methanol, dichloromethane, 1,2-dichloroethane or acetic acid. The halogen in the α-position, preferably Cl or Br, can be subsequently substituted by an imidazole of formula (X) to arrive at a compound of formula (IX). Preferably, this transformation is being conducted in the presence of a base, such as Na₂CO₃, K₂CO₃, K₃PO₄, Cs₂CO₃, NaOH, KOtBu, NaH or mixtures thereof, preferably in the presence of an organic solvent, such as tetrahydrofuran, dimethylformamide, acetonitrile or toluene.

Imidazoles (IX) can then be converted into the corresponding dioxolanes (I) by reaction with the corresponding diol, preferably in the presence of Lewis or Broensted acid catalyst, such as for instance para-toluenesulfonic acid, triflic acid, tetrabutylammonium tribromide (R. Gopinath, Sk. J. Hague, B. K. Patel, J. Org. Chem., 2002, 67, 5842-5845.), zirconium tetrachloride (H. Firouzabadi, N. Iranpoor, B. Karimi, Synlett, 1999, 321-323) or cerium(III) trifluoromethanesulfonate (F. Ono, H. Takenaka, T Fujikawa, M. Mori, T. Sato, Synthesis, 2009, 1318-1322.), optionally in the presence of trialkyl orthoformate. The reactions are preferably carried out in an organic solvent such as toluene, cyclohexane, DMF, ethyl acetate, DMSO, methanol, or dichloromethane.

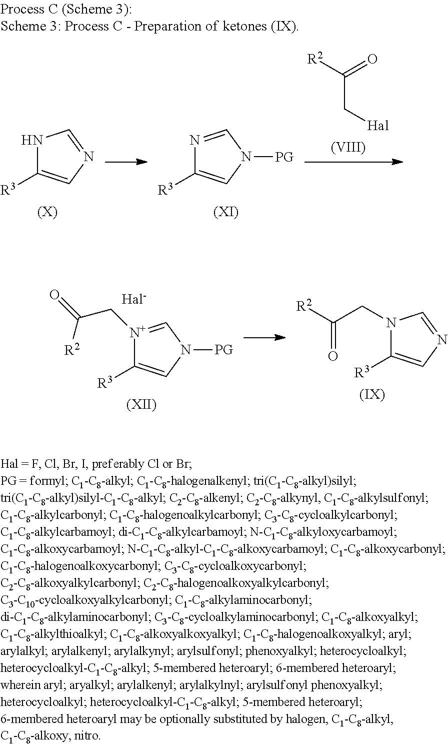

Alternatively, ketones of formula (IX) can be obtained from imidazoles of formula (X) according to the method described in Scheme 3.

Imidazoles (X), which are commercially available or can be obtained by means of methods described in the literature, can be converted into imidazoles of formula (XI) by means of methods described in the literature (see e.g “Protective groups in organic synthesis”, Wiley Interscience, 1999; 3^{rd} edition, T. Greene & P. Wuts, p. 615-632 and references cited therein, Journal of organic chemistry (2013), 78, 12220-12223). The reaction is optionally conducted in the presence of a base, such as potassium carbonate, triethylamine, and/or potassium tert-butoxide, optionally in the presence of a Lewis acid, such as magnesium dichloride or BF₃/Et₂O, optionally in the presence of a metal oxide, such as zinc oxide or barium oxide.

The imidazoles of formula (XI) can consequently be converted into imidazolium salts of formula (XII) by means of methods described in the literature (see e.g “Protective groups in organic synthesis”, Wiley Interscience, 1999; 3^{rd} edition, T. Greene & P. Wuts, p. 615-632 and references cited therein, Journal of organic chemistry (2013), 78, 12220-12223). The reaction is optionally conducted in the presence of a base, such as potassium carbonate, triethylamine, and/or potassium tert-butoxide, optionally in the presence of a Lewis acid, such as magnesium dichloride or BF₃/Et₂O, optionally in the presence of a metal oxide, such as zinc oxide or barium oxide.

Finally, imidazolium salts of formula (XII) can be converted into ketones of formula (IX) by means of methods described in the literature (see e.g “Protective groups in organic synthesis”, Wiley Interscience, 1999; 3^{rd} edition, T. Greene & P. Wuts, p. 615-632 and references cited therein, Journal of organic chemistry (2013), 78, 12220-12223).

As the solvent, all common solvents inert under the reaction conditions, such as for example nitriles (such as e.g. acetonitrile, propionitrile) or alcohols (such as e.g. methanol, ethanol), can be used and the reaction can be effected in mixtures of two or more of these solvents.

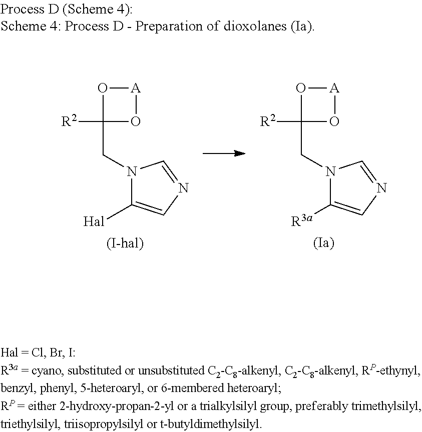

The compounds (I-hal) obtainable for example according to process B can be converted by means of methods described in the literature to the corresponding compounds (Ia) via a coupling reaction with either:
for R^{3a}=cyano: a cyanide source such as KCN, NaCN, Zn(CN)₂, CuCN, K₃Fe(CN)₆, K₄Fe(CN)₆ or acetone cyanohydrin, which are commercially available (see e.g. P. Anbarasan, T. Schareina, M. Beller, Chem. Soc. Rev. 2011, 40, 5049; F. Burg, J. Egger, J. Deutsch, N. Guimond, Org. Process Res. Dev. 2016, 20, 1540-1545; J. R. Coombs, K. J. Fraunhoffer, J. M. Stevens, S. R. Wisniewski, M. Yu, J. Org. Chem. 2017, 82, 7040-7044 and references therein).
for R^{3a}=C₂-C₈-alkynyl or R^{P}-ethynyl: a terminal alkyne which is commercially available or can be prepared by known processes (see e.g. R. Chinchilla, C. Najera Chem. Soc. Rev., 2011, 40, 5084-5121 and references therein) or a suitable precursor such as calcium carbide.
for R^{3a}=C₂-C₈-alkenyl: an alkene which is commercially available or can be prepared by known processes (see e.g. I. P. Beletskaya, A. V. Cheprakov, Chem. Rev. 2000, 100, 3009-3066 and references therein)
for R^{3a}=C₂-C₈-alkyl, benzyl, phenyl, 5-heteroalyl, or 6-membered heteroalyl: a boronic acid of R^{3a}, boronic ester, borinic acid, borane or trifluoroborate salt which are commercially available or can be prepared by known processes (see e.g. C. Torborg, M. Beller, Adv. Synth. Catal. 2009, 351, 3027; G. A. Molander, D. L. Sandrock, Curr. Opin. Drug. Discov. Devel. 2009 12(6):811-823; F. S. Han, Chem. Soc. Rev., 2013, 42, 5270-98 and references therein),

In each case preferably in the presence of one or several additives and a catalyst.

The catalyst, preferably a transition metal catalyst, may consist of
either a preformed copper, nickel or palladium complex [e.g. tetrakis-(triphenylphosphine) palladium(0), bis-(triphenylphosphine) palladium dichloride (II), tris(dibenzylideneacetone) dipalladium(0), bis(dibenzylideneacetone) palladium(0), allylpalladium(II) chloride dimer, palladium(π-cinnamyl) chloride dimer, 1,1′-bis(diphenylphosphino)ferrocene-palladium (II) chloride, [(TMEDA)Ni(o-tolyl)Cl] or bis(1,5-cyclooctadiene)nickel(0)];
or a mixture of a copper, nickel or palladium salt [e.g. palladium (II) chloride, palladium (II) acetate, nickel(II) bromide 2-methoxyethyl ether complex, nickel(II) bromide ethylene glycol dimethyl ether complex] with a ligand or salt [e.g. triphenylphosphine, tri-tert-butylphosphine, tri-tert-butylphosphonium tetrafluoroborate, tricyclohexylphosphine, 2-(dicyclohexylphosphino)biphenyl, 2-(di-tert-butylphosphino)biphenyl, 2-(dicyclohexylphosphino)-2′-(N,N-dimethylamino)biphenyl, 2-(tert-butylphosphino)-2′-(N,N-dimethylamino)biphenyl, 2-di-tert-butylphosphino-2′,4′,6′-triisopropylbiphenyl 2-dicyclohexylphosphino-2′,4′,6′-triisopropylbiphenyl, 2-dicyclohexylphosphino-2,6′-dimethoxybiphenyl, 2-dicyclohexylphosphino-2′,6′-diisopropoxybiphenyl, triphenyl-phosphine, tris-(o-tolyl)phosphine, sodium 3-(diphenylphosphino)benzenesulfonate, tris-2-(methoxy-phenyl)phosphine, 2,2′-bis (diphenylphosphino)-1,1′-binaphthyl, 1,4-bis(diphenylphosphino)butane, 1,2-bis(diphenylphosphino) ethane, 1,4-bis(dicyclohexylphosphino)butane, 1,2-bis(dicyclohexylphosphino)-ethane, 2-(dicyclohexylphosphino)-2′-(N,N-dimethylamino)-biphenyl, 1,1′-bis(diphenylphosphino)-ferrocene, (R)-(−)-1-[(S)-2-diphenyl-phosphino)ferrocenyl]ethyldicyclohexylphosphine, tris-(2,4-tert-butyl-phenyl)phosphite, di(1-adamantyl)-2-morpholinophenylphosphine or 1,3-bis(2,4,6-trimethylphenyl)imidazolium chloride].

It is also advantageous to choose the appropriate catalyst and/or ligand from commercial sources such as the catalogues “Metal Catalysts for Organic Synthesis” by Strem Chemicals or “Phosphorous Ligands and Compounds” by Strem Chemicals.

Suitable additives for carrying out Process D can be inorganic and organic bases which are customary for such reactions. Preference is given to using alkaline earth metal or alkali metal hydroxides, such as sodium hydroxide, calcium hydroxide, potassium hydroxide or other ammonium hydroxide derivatives; alkaline earth metal, alkali metal or ammonium fluorides such as potassium fluoride, caesium fluoride or tetrabutylammonium fluoride; alkaline earth metal or alkali metal carbonates, such as sodium carbonate, potassium carbonate, potassium bicarbonate, sodium bicarbonate or caesium carbonate; alkali metal or alkaline earth metal acetates, such as sodium acetate, lithium acetate, potassium acetate or calcium acetate; alkali metal or alkaline earth metal phosphate, such as tripotassium phosphate; alkali metal alcoholates, such as potassium tert-butoxide or sodium tert-butoxide; tertiary amines, such as trimethylamine, triethylamine, tributylamine, N,N-dicyclohexylmethylamine, N,N-diisopropylethylamine, N-methylpiperidine, N,N-dimethylaminopyridine, diazabicyclooctane (DABCO), diazabicyclononene (DBN) or diazabicycloundecene (DBU); and also aromatic bases, such as pyridine, picolines, lutidines or collidines. In the specific cases wherein R^{3a}=C₂-C₈-alkynyl, an additional additive can be a copper(I) salt, such as e.g. CuI or CuBr usually in substoichiometric amount.

Suitable solvents for carrying out process D can be customary inert organic solvents. Preference is given to using optionally halogenated, aliphatic, alicyclic or aromatic hydrocarbons, such as petroleum ether, pentane, hexane, heptane, cyclohexane, methylcyclohexane, benzene, toluene, xylene or decalin; chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 2-methyltetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; ureas, such as 1,3-dimethyl-3,4,5,6-tetrahydro-2(1H)-pyrimidinone; esters, such as methyl acetate or ethyl acetate, sulfoxides, such as dimethyl sulfoxide, or sulfones, such as sulfolane; and a mixture thereof.

It can also be advantageous to carry out process D in the presence of a co-solvent such as water or an alcohol, such as methanol, ethanol, propanol, isopropanol or tent-butanol

Process D may be performed in an inert atmosphere such as argon or nitrogen atmosphere. When carrying out process D, 1 mole or an excess of compound of formula (Ia) and from 1 to 5 moles of base and from 0.01 to 20 mole percent of a palladium complex can be employed per mole of boronic acid, boronic ester or alkynes. It is also possible to employ the reaction components in other ratios. Work-up is carried out by known methods.

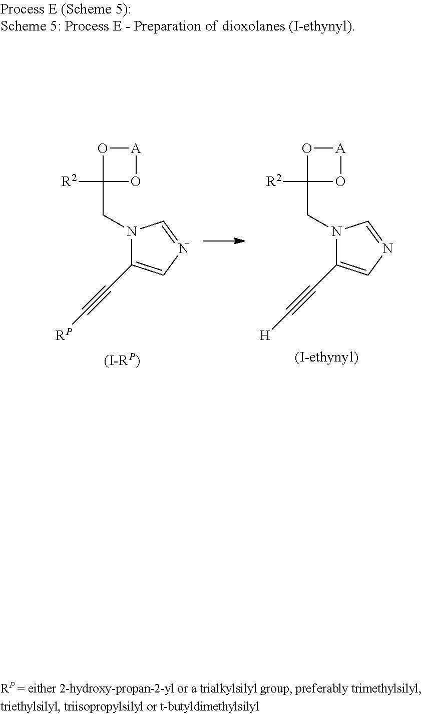

The compounds (I-R^{P}) obtainable for example according to process D can be converted by means of methods described in the literature (see e.g. T. W. Greene, P. G. M. Wuts, “Protective Groups in Organic Synthesis, 3^{rd} Edition”, 1999, Wiley Interscience, John Wiley & Sons Inc., New York) to the corresponding compounds (1-ethynyl) via a deprotection step, consisting of either:
for R^{P}=trimethylsilyl, triethylsilyl, triisopropylsilyl, t-butyldimethylsilyl: by treatment with a fluoride source (e.g. tetra-n-butylammonium fluoride, KF or CsF) or an organic or inorganic base (e.g. potassium or sodium carbonate, potassium or sodium hydroxide, potassium tert-butoxide, methyllithium or n-butyllithium),
for R^{P}=2-hydroxy-propan-2-yl: by treatment with an organic or inorganic base (e.g. potassium or sodium carbonate, potassium or sodium hydroxide, potassium tert-butoxide, methyllithium or n-butyllithium),

in a suitable solvent. Suitable solvents for carrying out process E can be customary inert organic solvents. Preference is given to using optionally halogenated, aliphatic, alicyclic or aromatic hydrocarbons, such as petroleum ether, pentane, hexane, heptane, cyclohexane, methylcyclohexane, benzene, toluene, xylene or decalin; chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 2-methyltetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; ureas, such as 1,3-dimethyl-3,4,5,6-tetrahydro-2(1H)-pyrimidinone; esters, such as methyl acetate or ethyl acetate, sulfoxides, such as dimethyl sulfoxide, or sulfones, such as sulfolane; and a mixture thereof. It can also be advantageous to carry out process E in the presence of a co-solvent such as water or an alcohol such as methanol, ethanol, propanol, isopropanol or tent-butanol

The preferred compounds of the formulae (I-1), (I-1-Q-I-1), (I-1-Q-I-2) and (I-1-Q-I-3) can also be obtained according to the processes A to E according to the invention. Unless indicated otherwise, the radicals A, R¹, R² R³ and Q have the meanings given above for the compounds of formulae (I-1), (I-1-Q-I-1), (I-1-Q-I-2) and (I-1-Q-I-3). These definitions apply not only to the end products of the formulae (I-1), (I-1-Q-I-1), (I-1-Q-I2) and (I-1-Q-I-3) but likewise to all intermediates.

General

The processes A to E according to the invention for preparing compounds of the formula (I) are optionally performed using one or more reaction auxiliaries.

Useful reaction auxiliaries are, as appropriate, inorganic or organic bases or acid acceptors. These preferably include alkali metal or alkaline earth metal acetates, amides, carbonates, hydrogencarbonates, hydrides, hydroxides or alkoxides, for example sodium acetate, potassium acetate or calcium acetate, lithium amide, sodium amide, potassium amide or calcium amide, sodium carbonate, potassium carbonate or calcium carbonate, sodium hydrogencarbonate, potassium hydrogencarbonate or calcium hydrogencarbonate, lithium hydride, sodium hydride, potassium hydride or calcium hydride, lithium hydroxide, sodium hydroxide, potassium hydroxide or calcium hydroxide, n-butyllithium, sec-butyllithium, tert-butyllithium, lithium diisopropylamide, lithium bis(trimethylsilyl)amide, sodium methoxide, ethoxide, n- or i-propoxide, n-, s- or t-butoxide or potassium methoxide, ethoxide, n- or i-propoxide, n-, s- or t-butoxide; and also basic organic nitrogen compounds, for example trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]-octane (DABCO), 1,5-diazabicyclo[4.3.0]-non-5-ene (DBN) or 1,8-diazabicyclo [5.4.0]-undec-7-ene (DBU).

Further useful reaction auxiliaries are, as appropriate, inorganic or organic acids. These preferably include inorganic acids, for example hydrogen fluoride, hydrogen chloride, hydrogen bromide and hydrogen iodide, sulphuric acid, phosphoric acid and nitric acid, and acidic salts such as NaHSO₄ and KHSO₄, or organic acids, for example, formic acid, carbonic acid and alkanoic acids such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, saturated or mono- or diunsaturated C₆-C₂₀ fatty acids, alkylsulphuric monoesters, alkylsulphonic acids (sulphonic acids having straight-chain or branched alkyl radicals having 1 to 20 carbon atoms), arylsulphonic acids or aryldisulphonic acids (aromatic radicals, such as phenyl and naphthyl, which bear one or two sulphonic acid groups), alkylphosphonic acids (phosphonic acids having straight-chain or branched alkyl radicals having 1 to 20 carbon atoms), arylphosphonic acids or aryldiphosphonic acids (aromatic radicals, such as phenyl and naphthyl, which bear one or two phosphonic acid radicals), where the alkyl and aryl radicals may bear further substituents, for example p-toluenesulphonic acid, salicylic acid, p-aminosalicylic acid, 2-phenoxybenzoic acid, 2-acetoxybenzoic acid, etc.

The processes A to E according to the invention are optionally performed using one or more diluents. Useful diluents are virtually all inert organic solvents. Unless otherwise indicated for the above described processes A to C, these preferably include aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers such as diethyl ether, dibutyl ether and methyl tert-butyl ether, glycol dimethyl ether and diglycol dimethyl ether, tetrahydrofuran and dioxane, ketones such as acetone, methyl ethyl ketone, methyl isopropyl ketone and methyl isobutyl ketone, esters, such as methyl acetate and ethyl acetate, nitriles, for example acetonitrile and propionitrile, amides, for example dimethylformamide, dimethylacetamide and N-methylpyrrolidone, and also dimethyl sulphoxide, tetramethylenesulphone and hexamethylphosphoramide and DMPU.

In the processes according to the invention, the reaction temperatures can be varied within a relatively wide range. In general, the temperatures employed are between −78° C. and 250° C., preferably temperatures between −78° C. and 150° C.

The reaction time varies as a function of the scale of the reaction and of the reaction temperature, but is generally between a few minutes and 48 hours.

The processes according to the invention are generally performed under standard pressure. However, it is also possible to work under elevated or reduced pressure.

For performance of the processes according to the invention, the starting materials required in each case are generally used in approximately equimolar amounts. However, it is also possible to use one of the components used in each case in a relatively large excess.

After a reaction has ended, the compounds are optionally separated from the reaction mixture by one of the customary separation techniques. If necessary, the compounds are purified by recrystallization or chromatography.

If appropriate, in the processes A to E according to the invention also salts and/or N-oxides of the starting compounds can be used.

The compounds of the formula (I) according to the invention can be converted into physiologically acceptable salts, e.g. as acid addition salts or metal salt complexes.

Depending on the nature of the substituents defined above, the compounds of the formula (I) have acidic or basic properties and can form salts, if appropriate also inner salts, or adducts with inorganic or organic acids or with bases or with metal ions. If the compounds of the formula (I) carry amino, alkylamino or other groups which induce basic properties, these compounds can be reacted with acids to give salts, or they are directly obtained as salts in the synthesis. If the compounds of the formula (I) carries hydroxyl, carboxyl or other groups which induce acidic properties, these compounds can be reacted with bases to give salts. Suitable bases are, for example, hydroxides, carbonates, bicarbonates of the alkali metals and alkaline earth metals, in particular those of sodium, potassium, magnesium and calcium, furthermore ammonia, primary, secondary and tertiary amines having (C₁-C₄)-alkyl groups, mono-, di- and trialkanolamines of (C₁-C₄)-alkanols, choline and also chlorocholine.

The salts obtainable in this manner also have fungicidal properties.

Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as NaHSO₄ and KHSO₄. Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, maleic acid, fumaric acid, tartaric acid, sorbic acid oxalic acid, alkylsulphonic acids (sulphonic acids having straight-chain or branched alkyl radicals of 1 to 20 carbon atoms), arylsulphonic acids or aryldisulphonic acids (aromatic radicals, such as phenyl and naphthyl, which carry one or two sulphonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulphonic acid, 1,5-naphthalenedisulphonic acid, salicylic acid, p-amino salicylic acid, 2-phenoxybenzoic acid, 2-acetoxybenzoic acid, etc.

Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others. Particular preference is given to the metal ions of the elements of the fourth period. Here, the metals can be present in various valencies that they can assume.

The acid addition salts of the compounds of the formula (I) can be obtained in a simple manner by customary methods for forming salts, for example by dissolving a compound of the formula (I) in a suitable inert solvent and adding the acid, for example hydrochloric acid, and be isolated in a known manner, for example by filtration, and, if required, be purified by washing with an inert organic solvent.

Suitable anions of the salts are those which are preferably derived from the following acids: hydrohalic acids, such as, for example, hydrochloric acid and hydrobromic acid, furthermore phosphoric acid, nitric acid and sulphuric acid.

The metal salt complexes of compounds of the formula (I) can be obtained in a simple manner by customary processes, for example by dissolving the metal salt in alcohol, for example ethanol, and adding the solution to the compound of the formula (I). Metal salt complexes can be isolated in a known manner, for example by filtration, and, if required, be purified by recrystallization.

Salts of the intermediates can also be prepared according to the processes mentioned above for the salts of compounds of formula (I).

N-oxides of compounds of the formula (I) or intermediates thereof can be obtained in a simple manner by customary processes, for example by N-oxidation with hydrogen peroxide (H₂O₂), peracids, for example peroxy sulfuric acid or peroxy carboxylic acids, such as meta-chloroperoxybenzoic acid or peroxymonosulfuric acid (Caro's acid).

Methods and Uses

The invention also relates to a method for controlling unwanted microorganisms, characterized in that the compounds of the formula (I) are applied to the microorganisms and/or in their habitat.

The invention further relates to seed which has been treated with at least one compound of the formula (I).

The invention finally provides a method for protecting seed against unwanted microorganisms by using seed treated with at least one compound of the formula (I).

The compounds of the formula (I) have potent microbicidal activity and can be used for control of unwanted microorganisms, such as fungi and bacteria, in crop protection and in the protection of materials.

The compounds of the formula (I) have very good fungicidal properties and can be used in crop protection, for example for control of Plasmodiophoromycetes, Oomycetes, Chytridiomycetes, Zygomycetes, Ascomycetes, Basidiomycetes and Deuteromycetes.

Bactericides can be used in crop protection, for example, for control of Pseudomonadaceae, Rhizobiaceae, Enterobacteriaceae, Corynebacteriaceae and Streptomycetaceae.

The compounds of the formula (I) can be used for curative or protective control of phytopathogenic fungi. The invention therefore also relates to curative and protective methods for controlling phytopathogenic fungi by the use of the inventive active ingredients or compositions, which are applied to the seed, the plant or plant parts, the fruit or the soil in which the plants grow.

Plants

All plants and plant parts can be treated in accordance with the invention. Plants are understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights. Plant parts are understood to mean all parts and organs of plants above and below the ground, such as shoot, leaf, flower and root, examples of which include leaves, needles, stalks, stems, flowers, fruit bodies, fruits and seeds, and also roots, tubers and rhizomes. The plant parts also include harvested material and vegetative and generative propagation material, for example cuttings, tubers, rhizomes, slips and seeds.

Plants which can be treated in accordance with the invention include the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelhferae sp., Cruciferae sp., Chenopodiaceae sp., Cucurbitaceae sp. (for example cucumber), Alliaceae sp. (for example leek, onion), Papilionaceae sp. (for example peas); major crop plants, such as Gramineae sp. (for example maize, turf, cereals such as wheat, lye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); useful plants and ornamental plants for gardens and wooded areas; and genetically modified varieties of each of these plants.

Pathogens

Non-limiting examples of pathogens of fungal diseases which can be treated in accordance with the invention include:

diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator;

diseases caused by rust disease pathogens, for example Gymnosporangium species, for example Gymnosporangium sabinae; Hemileia species, for example Hemileia vastatrix; Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus;

diseases caused by pathogens from the group of the Oomycetes, for example Albugo species, for example Albugo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum;

leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidial form: Drechslera, syn: Helminthosporium) or Cochliobolus miyabeanus; Colletotrichum species, for example Colletotrichum lindemuthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans; Magnaporthe species, for example Magnaporthe grisea; Microdochium species, for example Microdochium nivale; Mycosphaerella species, for example Mycosphaerella graminicola, Mycosphaerella arachidicola or Mycosphaerella fijiensis; Phaeosphaeria species, for example Phaeosphaeria nodorum; Pyrenophora species, for example Pyrenophora teres or Pyrenophora tritici repentis; Ramularia species, for example Ramularia collo-cygni or Ramularia areola; Rhynchosporium species, for example Rhynchosporium secalis; Septoria species, for example Septoria apii or Septoria lycopersici; Stagonospora species, for example Stagonospora nodorum; Typhula species, for example Typhula incarnata; Venturia species, for example Venturia inaequalis;

root and stem diseases caused, for example, by Corticium species, for example Corticium graminearum; Fusarium species, for example Fusarium oxysporum; Gaeumannomyces species, for example Gaeumannomyces graminis; Plasmodiophora species, for example Plasmodiophora brassicae; Rhizoctonia species, for example Rhizoctonia solani; Sarocladium species, for example Sarocladium olyzae; Sclerotium species, for example Sclerotium oryzae; Tapesia species, for example Tapesia acuformis; Thielaviopsis species, for example Thielaviopsis basicola;

ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Stagnospora species, for example Stagnospora nodorum;

diseases caused by smut fungi, for example Sphacelotheca species, for example Sphacelotheca reiliana; Tilletia species, for example Tilletia caries or Tilletia controversa; Urocystis species, for example Urocystis occulta; Ustilago species, for example Ustilago nuda;

fruit rot caused, for example, by Aspergillus species, for example Aspergillus flavus; Botrytis species, for example Botrytis cinerea; Penicillium species, for example Penicillium expansum or Penicillium purpurogenum; Rhizopus species, for example Rhizopus stolonifer; Sclerotinia species, for example Sclerotinia sclerotiomm; Verticilium species, for example Verticilium alboatmm;

seed- and soil-borne rot and wilt diseases, and also diseases of seedlings, caused, for example, by Alternaria species, for example Alternaria brassicicola; Aphanomyces species, for example Aphanomyces euteiches; Ascochyta species, for example Ascochyta lentis; Aspergillus species, for example Aspergillus flavus; Cladosporium species, for example Cladosporium herbarum; Cochliobolus species, for example Cochliobolus sativus (conidial form: Drechslera, Bipolaris Syn: Helminthosporium); Colletotrichum species, for example Colletotrichum coccodes; Fusarium species, for example Fusarium culmorum; Gibberella species, for example Gibberella zeae; Macrophomina species, for example Macrophomina phaseolina; Microdochium species, for example Microdochium nivale; Monographella species, for example Monographella nivalis; Penicillium species, for example Penicillium expansum; Phoma species, for example Phoma lingam; Phomopsis species, for example Phomopsis sojae; Phytophthora species, for example Phytophthora cactorum; Pyrenophora species, for example Pyrenophora graminea; Pyricularia species, for example Pyricularia oryzae; Pythium species, for example Pythium ultimum; Rhizoctonia species, for example Rhizoctonia solani; Rhizopus species, for example Rhizopus olyzae; Sclerotium species, for example Sclerotium rolfsii; Septoria species, for example Septoria nodorum; Typhula species, for example Typhula incarnata; Verticillium species, for example Verticillium dahliae;

cancers, galls and witches' broom caused, for example, by Nectria species, for example Nectria galligena;

wilt diseases caused, for example, by Monilinia species, for example Monilinia laxa;

deformations of leaves, flowers and fruits caused, for example, by Exobasidium species, for example Exobasidium vexans; Taphrina species, for example Taphrina deformans;

degenerative diseases in woody plants, caused, for example, by Esca species, for example Phaeomoniella chlamydospora, Phaeoacremonium aleophilum or Fomitiporia mediterranea; Ganoderma species, for example Ganoderma boninense;

diseases of flowers and seeds caused, for example, by Botiytis species, for example Botiytis cinerea;

diseases of plant tubers caused, for example, by Rhizoctonia species, for example Rhizoctonia solani; Helminthosporium species, for example Helminthosporium solani;

diseases caused by bacterial pathogens, for example Xanthomonas species, for example Xanthomonas campestris pv. olyzae; Pseudomonas species, for example Pseudomonas syringae pv. lachiymans; Erwinia species, for example Erwinia amylovora.

Preference is given to controlling the following diseases of soya beans:

Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaemlina leaf spot (Leptosphaemlina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botiyosum), target spot (Coiynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxyspomm, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolomm), stem canker (Diaporthe phaseolomm var. caulivora), phytophthora rot (Phytophthora megasperma), brown stem rot (Phialophora gregata), pythium rot (Pythium aphanidermatum, Pythium irregulare, Pythium debalyanum, Pythium myriotylum, Pythium ultimum), rhizoctonia root rot, stem decay, and damping-off (Rhizoctonia solani), sclerotinia stem decay (Sclerotinia sclerotiomm), sclerotinia southern blight (Sclerotinia rolfsii), thielaviopsis root rot (Thielaviopsis basicola).

Plant Growth Regulation

In some cases, the compounds of the formula (I) can, at particular concentrations or application rates, also be used as growth regulators or agents to improve plant properties, or as microbicides, for example as fungicides, antimycotics, bactericides, viricides (including compositions against viroids) or as compositions against MLO (Mycoplasma-like organisms) and RLO (Rickettsia-like organisms).

The compounds of the formula (I) intervene in physiological processes of plants and can therefore also be used as plant growth regulators. Plant growth regulators may exert various effects on plants. The effect of the substances depends essentially on the time of application in relation to the developmental stage of the plant, and also on the amounts of active ingredient applied to the plants or their environment and on the type of application. In each case, growth regulators should have a particular desired effect on the crop plants.

Growth regulating effects, comprise earlier germination, better emergence, more developed root system and/or improved root growth, increased ability of tillering, more productive tillers, earlier flowering, increased plant height and/or biomass, shorting of stems, improvements in shoot growth, number of kernels/ear, number of ears/m², number of stolons and/or number of flowers, enhanced harvest index, bigger leaves, less dead basal leaves, improved phyllotaxy, earlier maturation/earlier fruit finish, homogenous riping, increased duration of grain filling, better fruit finish, bigger fruit/vegetable size, sprouting resistance and reduced lodging.

Increased or improved yield is referring to total biomass per hectare, yield per hectare, kernel/fruit weight, seed size and/or hectolitre weight as well as to improved product quality, comprising:

improved processability relating to size distribution (kernel, fruit, etc.), homogenous riping, grain moisture, better milling, better vinification, better brewing, increased juice yield, harvestability, digestibility, sedimentation value, falling number, pod stability, storage stability, improved fiber length/strength/uniformity, increase of milk and/or meet quality of silage fed animals, adaptation to cooking and frying;

further comprising improved marketability relating to improved fruit/grain quality, size distribution (kernel, fruit, etc.), increased storage/shelf-life, firmness/softness, taste (aroma, texture, etc.), grade (size, shape, number of berries, etc.), number of berries/fruits per bunch, crispness, freshness, coverage with wax, frequency of physiological disorders, colour, etc.;

further comprising increased desired ingredients such as e.g. protein content, fatty acids, oil content, oil quality, aminoacid composition, sugar content, acid content (pH), sugar/acid ratio (Brix), polyphenols, starch content, nutritional quality, gluten content/index, energy content, taste, etc.;

and further comprising decreased undesired ingredients such as e.g. less mycotoxines, less aflatoxins, geosmin level, phenolic aromas, lacchase, polyphenol oxidases and peroxidases, nitrate content etc.

Plant growth-regulating compounds can be used, for example, to slow down the vegetative growth of the plants. Such growth depression is of economic interest, for example, in the case of grasses, since it is thus possible to reduce the frequency of grass cutting in ornamental gardens, parks and sport facilities, on roadsides, at airports or in fruit crops. Also of significance is the inhibition of the growth of herbaceous and woody plants on roadsides and in the vicinity of pipelines or overhead cables, or quite generally in areas where vigorous plant growth is unwanted.

Also important is the use of growth regulators for inhibition of the longitudinal growth of cereal. This reduces or completely eliminates the risk of lodging of the plants prior to harvest. In addition, growth regulators in the case of cereals can strengthen the culm, which also counteracts lodging The employment of growth regulators for shortening and strengthening culms allows the deployment of higher fertilizer volumes to increase the yield, without any risk of lodging of the cereal crop.

In many crop plants, vegetative growth depression allows denser planting, and it is thus possible to achieve higher yields based on the soil surface. Another advantage of the smaller plants obtained in this way is that the crop is easier to cultivate and harvest.

Reduction of the vegetative plant growth may also lead to increased or improved yields because the nutrients and assimilates are of more benefit to flower and fruit formation than to the vegetative parts of the plants.

Alternatively, growth regulators can also be used to promote vegetative growth. This is of great benefit when harvesting the vegetative plant parts. However, promoting vegetative growth may also promote generative growth in that more assimilates are formed, resulting in more or larger fruits.

Furthermore, beneficial effects on growth or yield can be achieved through improved nutrient use efficiency, especially nitrogen (N)-use efficiency, phosphorus (P)-use efficiency, water use efficiency, improved transpiration, respiration and/or CO2 assimilation rate, better nodulation, improved Ca-metabolism etc.

Likewise, growth regulators can be used to alter the composition of the plants, which in turn may result in an improvement in quality of the harvested products. Under the influence of growth regulators, parthenocarpic fruits may be formed. In addition, it is possible to influence the sex of the flowers. It is also possible to produce sterile pollen, which is of great importance in the breeding and production of hybrid seed.

Use of growth regulators can control the branching of the plants. On the one hand, by breaking apical dominance, it is possible to promote the development of side shoots, which may be highly desirable particularly in the cultivation of ornamental plants, also in combination with an inhibition of growth. On the other hand, however, it is also possible to inhibit the growth of the side shoots. This effect is of particular interest, for example, in the cultivation of tobacco or in the cultivation of tomatoes.

Under the influence of growth regulators, the amount of leaves on the plants can be controlled such that defoliation of the plants is achieved at a desired time. Such defoliation plays a major role in the mechanical harvesting of cotton, but is also of interest for facilitating harvesting in other crops, for example in viticulture. Defoliation of the plants can also be undertaken to lower the transpiration of the plants before they are transplanted.

Furthermore, growth regulators can modulate plant senescence, which may result in prolonged green leaf area duration, a longer grain filling phase, improved yield quality, etc.

Growth regulators can likewise be used to regulate fruit dehiscence. On the one hand, it is possible to prevent premature fruit dehiscence. On the other hand, it is also possible to promote fruit dehiscence or even flower abortion to achieve a desired mass (“thinning”) In addition it is possible to use growth regulators at the time of harvest to reduce the forces required to detach the fruits, in order to allow mechanical harvesting or to facilitate manual harvesting.

Growth regulators can also be used to achieve faster or else delayed ripening of the harvested material before or after harvest. This is particularly advantageous as it allows optimal adjustment to the requirements of the market. Moreover, growth regulators in some cases can improve the fruit colour. In addition, growth regulators can also be used to synchronize maturation within a certain period of time. This establishes the prerequisites for complete mechanical or manual harvesting in a single operation, for example in the case of tobacco, tomatoes or coffee.

By using growth regulators, it is additionally possible to influence the resting of seed or buds of the plants, such that plants such as pineapple or ornamental plants in nurseries, for example, germinate, sprout or flower at a time when they are normally not inclined to do so. In areas where there is a risk of frost, it may be desirable to delay budding or germination of seeds with the aid of growth regulators, in order to avoid damage resulting from late frosts.

Finally, growth regulators can induce resistance of the plants to frost, drought or high salinity of the soil. This allows the cultivation of plants in regions which are normally unsuitable for this purpose.

Resistance Induction/Plant Health and Other Effects

The compounds of the formula (I) also exhibit a potent strengthening effect in plants. Accordingly, they can be used for mobilizing the defences of the plant against attack by undesirable microorganisms.

Plant-strengthening (resistance-inducing) substances in the present context are substances capable of stimulating the defence system of plants in such a way that the treated plants, when subsequently inoculated with undesirable microorganisms, develop a high degree of resistance to these microorganisms.

Further, in context with the present invention plant physiology effects comprise the following:

Abiotic stress tolerance, comprising tolerance to high or low temperatures, drought tolerance and recovery after drought stress, water use efficiency (correlating to reduced water consumption), flood tolerance, ozone stress and UV tolerance, tolerance towards chemicals like heavy metals, salts, pesticides etc.

Biotic stress tolerance, comprising increased fungal resistance and increased resistance against nematodes, viruses and bacteria. In context with the present invention, biotic stress tolerance preferably comprises increased fungal resistance and increased resistance against nematodes.

Increased plant vigor, comprising plant health/plant quality and seed vigor, reduced stand failure, improved appearance, increased recovery after periods of stress, improved pigmentation (e.g. chlorophyll content, stay-green effects, etc.) and improved photosynthetic efficiency.

Mycotoxins

In addition, the compounds of the formula (I) can reduce the mycotoxin content in the harvested material and the foods and feeds prepared therefrom. Mycotoxins include particularly, but not exclusively, the following: deoxynivalenol (DON), nivalenol, 15-Ac-DON, 3-Ac-DON, T2- and HT2-toxin, fumonisins, zearalenon, moniliformin, fusarin, diaceotoxyscirpenol (DAS), beauvericin, enniatin, fusaroproliferin, fusarenol, ochratoxins, patulin, ergot alkaloids and aflatoxins which can be produced, for example, by the following fungi: Fusarium spec., such as F. acuminatum, F. asiaticum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides etc., and also by Aspergillus spec., such as A. flavus, A. parasiticus, A. nomius, A. ochraceus, A. clavatus, A. terreus, A. versicolor, Penicillium spec., such as P. verrucosum, P. viridicatum, P. citrinum, P. expansum, P. claviforme, P. roqueforti, Claviceps spec., such as C. purpurea, C. fusiformis, C. paspali, C. africana, Stachybotrys spec. and others.

Material Protection

The compounds of the formula (I) can also be used in the protection of materials, for protection of industrial materials against attack and destruction by phytopathogenic fungi.

In addition, the compounds of the formula (I) can be used as antifouling compositions, alone or in combinations with other active ingredients.

Industrial materials in the present context are understood to mean inanimate materials which have been prepared for use in industry. For example, industrial materials which are to be protected by inventive compositions from microbial alteration or destruction may be adhesives, glues, paper, wallpaper and board/cardboard, textiles, carpets, leather, wood, fibers and tissues, paints and plastic articles, cooling lubricants and other materials which can be infected with or destroyed by microorganisms. Parts of production plants and buildings, for example cooling-water circuits, cooling and heating systems and ventilation and air-conditioning units, which may be impaired by the proliferation of microorganisms may also be mentioned within the scope of the materials to be protected. Industrial materials within the scope of the present invention preferably include adhesives, sizes, paper and card, leather, wood, paints, cooling lubricants and heat transfer fluids, more preferably wood.

The compounds of the formula (I) may prevent adverse effects, such as rotting, decay, discoloration, decoloration or formation of mould.

In the case of treatment of wood the compounds of the formula (I) may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting a composition according to the invention; this includes for example direct application, spraying, dipping, injection or any other suitable means.

In addition, the compounds of the formula (I) can be used to protect objects which come into contact with saltwater or brackish water, especially hulls, screens, nets, buildings, moorings and signalling systems, from fouling

The compounds of the formula (I) can also be employed for protecting storage goods. Storage goods are understood to mean natural substances of vegetable or animal origin or processed products thereof which are of natural origin, and for which long-term protection is desired. Storage goods of vegetable origin, for example plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The inventive compositions may prevent adverse effects, such as rotting, decay, discoloration, decoloration or formation of mould.

Microorganisms capable of degrading or altering the industrial materials include, for example, bacteria, fungi, yeasts, algae and slime organisms. The compounds of the formula (I) preferably act against fungi, especially moulds, wood-discoloring and wood-destroying fungi (Ascomycetes, Basidiomycetes, Deuteromycetes and Zygomycetes), and against slime organisms and algae. Examples include microorganisms of the following genera: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride; Ophiostoma spp., Ceratocystis spp., Humicola spp., Petriella spp., Trichurus spp., Coriolus spp., Gloeophyllum spp., Pleurotus spp., Poria spp., Serpula spp. and Tyromyces spp., Cladosporium spp., Paecilomyces spp. Mucor spp., Escherichia, such as Escherichia coli; Pseudomonas, such as Pseudomonas aeruginosa; Staphylococcus, such as Staphylococcus aureus, Candida spp. and Saccharomyces spp., such as Saccharomyces cerevisae.

Formulations

The present invention further relates to a composition for controlling unwanted microorganisms, comprising at least one of the compounds of the formula (I). These are preferably fungicidal compositions which comprise agriculturally suitable auxiliaries, solvents, carriers, surfactants or extenders.

According to the invention, a carrier is a natural or synthetic, organic or inorganic substance with which the active ingredients are mixed or combined for better applicability, in particular for application to plants or plant parts or seed. The carrier, which may be solid or liquid, is generally inert and should be suitable for use in agriculture.

Useful solid carriers include: for example ammonium salts and natural rock flours, such as kaolins, clays, talc, chalk, quartz, attapulgite, montmorillonite or diatomaceous earth, and synthetic rock flours, such as finely divided silica, alumina and silicates; useful solid carriers for granules include: for example, crushed and fractionated natural rocks such as calcite, marble, pumice, sepiolite and dolomite, and also synthetic granules of inorganic and organic flours, and granules of organic material such as paper, sawdust, coconut shells, maize cobs and tobacco stalks; useful emulsifiers and/or foam-formers include: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE and/or -POP ethers, acid and/or POP POE esters, alkylaryl and/or POP POE ethers, fat and/or POP POE adducts, POE- and/or POP-polyol derivatives, POE- and/or POP-sorbitan or -sugar adducts, alkyl or aryl sulphates, alkyl- or arylsulphonates and alkyl or aryl phosphates or the corresponding PO-ether adducts. Additionally suitable are oligo- or polymers, for example those derived from vinylic monomers, from acrylic acid, from EO and/or PO alone or in combination with, for example, (poly)alcohols or (poly)amines. It is also possible to use lignin and its sulphonic acid derivatives, unmodified and modified celluloses, aromatic and/or aliphatic sulphonic acids and also their adducts with formaldehyde.

The active ingredients can be converted to the customary formulations, such as solutions, emulsions, wettable powders, water- and oil-based suspensions, powders, dusts, pastes, soluble powders, soluble granules, granules for broadcasting, suspoemulsion concentrates, natural products impregnated with active ingredient, synthetic substances impregnated with active ingredient, fertilizers and also microencapsulations in polymeric substances.

The active ingredients can be applied as such, in the form of their formulations or the use forms prepared therefrom, such as ready-to-use solutions, emulsions, water- or oil-based suspensions, powders, wettable powders, pastes, soluble powders, dusts, soluble granules, granules for broadcasting, suspoemulsion concentrates, natural products impregnated with active ingredient, synthetic substances impregnated with active ingredient, fertilizers and also microencapsulations in polymeric substances. Application is accomplished in a customary manner, for example by watering, spraying, atomizing, broadcasting, dusting, foaming, spreading-on and the like. It is also possible to deploy the active ingredients by the ultra-low volume method or to inject the active ingredient preparation/the active ingredient itself into the soil. It is also possible to treat the seed of the plants.

The formulations mentioned can be prepared in a manner known per se, for example by mixing the active ingredients with at least one customary extender, solvent or diluent, emulsifier, dispersant and/or binder or fixing agent, wetting agent, a water repellent, if appropriate siccatives and UV stabilizers and if appropriate dyes and pigments, antifoams, preservatives, secondary thickeners, stickers, gibberellins and also other processing auxiliaries.

The present invention includes not only formulations which are already ready for use and can be deployed with a suitable apparatus to the plant or the seed, but also commercial concentrates which have to be diluted with water prior to use.

The compounds of the formula (I) may be present as such or in their (commercial) formulations and in the use forms prepared from these formulations as a mixture with other (known) active ingredients, such as insecticides, attractants, sterilants, bactericides, acaricides, nematicides, fungicides, growth regulators, herbicides, fertilizers, safeners and/or semiochemicals.

The auxiliaries used may be those substances which are suitable for imparting particular properties to the composition itself or and/or to preparations derived therefrom (for example spray liquors, seed dressings), such as certain technical properties and/or also particular biological properties. Typical auxiliaries include: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which may optionally also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

Liquefied gaseous extenders or carriers are understood to mean liquids which are gaseous at standard temperature and under standard pressure, for example aerosol propellants such as halohydrocarbons, or else butane, propane, nitrogen and carbon dioxide.

In the formulations it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids. Further additives may be mineral and vegetable oils.

If the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Compositions comprising compounds of the formula (I) may additionally comprise further components, for example surfactants. Suitable surfactants are emulsifiers and/or foam formers, dispersants or wetting agents having ionic or nonionic properties, or mixtures of these surfactants. Examples thereof are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydroly sates, lignosulphite waste liquors and methylcellulose. The presence of a surfactant is necessary if one of the active ingredients and/or one of the inert carriers is insoluble in water and when application is effected in water. The proportion of surfactants is between 5 and 40 per cent by weight of the inventive composition.

It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

Further additives may be perfumes, mineral or vegetable, optionally modified oils, waxes and nutrients (including trace nutrients), such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability.

If appropriate, other additional components may also be present, for example protective colloids, binders, adhesives, thickeners, thixotropic substances, penetrants, stabilizers, sequestering agents, complex formers. In general, the active ingredients can be combined with any solid or liquid additive commonly used for formulation purposes.

The formulations contain generally between 0.05 and 99% by weight, 0.01 and 98% by weight, preferably between 0.1 and 95% by weight, more preferably between 0.5 and 90% of active ingredient, most preferably between 10 and 70 per cent by weight.

The formulations described above can be used for controlling unwanted microorganisms, in which the compositions comprising compounds of the formula (I) are applied to the microorganisms and/or in their habitat.

Mixtures

Compounds of the formula (I) can be used as such or in formulations thereof and can be mixed with known fungicides, bactericides, acaricides, nematicides or insecticides, in order thus to broaden, for example, the activity spectrum or to prevent development of resistance.

Useful mixing partners include, for example, known fungicides, insecticides, acaricides, nematicides or else bactericides (see also Pesticide Manual, 14th ed.).

A mixture with other known active ingredients, such as herbicides, or with fertilizers and growth regulators, safeners and/or semiochemicals, is also possible.

Hence, the invention further relates to mixtures and formulations, comprising at least one compound of formula (I) and at least a further active compound, preferably selected from fungicides, bactericides, acaricides, nematicides, insecticides, herbicides, fertilizers, growth regulators, safeners and/or semiochemicals, more preferably from fungicides, insecticides, herbicides, growth regulators and/or safeners, most preferably from fungicides.

Preferably the at least one further active compound is a fungicide selected from the following groups
(1) inhibitors of the ergosterol synthesis,
(2) inhibitors of the respiratory chain at complex I or II,
(3) inhibitors of the respiratory chain at complex III,
(4) inhibitors of the mitosis and cell division,
(5) compounds capable of having a multisite action,
(6) compounds capable of inducing a host defense,
(7) inhibitors of the amino acid and/or protein biosynthesis,
(8) inhibitors of the ATP production,
(9) inhibitors of the cell wall synthesis,
(10) inhibitors of the lipid and membrane synthesis,
(11) inhibitors of the melanin biosynthesis,
(12) inhibitors of the nucleic acid synthesis,
(13) inhibitors of the signal transduction,
(14) compounds capable of acting as uncoupler,
(15) other fungicides.

More preferably the at least one further active compound is selected from the group consisting of (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) Pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro -4-(4-chlorophenoxy)phenyl]-4-methyl-1,3-dioxolan-2-yllmethyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel (2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy -2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy -2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy -2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy -2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy -2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy -2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N-(2,5-dimethyl-4-{3-[(2,2,2-trifluoroethy)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) Mefentrifluconazole, (1.082) Ipfentrifluconazole, (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) Isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of syn-epimeric racemate 1RS,4SR,9RS and anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) Pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3 S)-7-fluoro-1,1,3-trimethyl-2,3-dihydro -1H-inden-4-yl]-1-methyl-1H-pyrazole-4-carboxamide, (2.033) 5,8-difluoro-N-(2-(2-fluoro-4-{[4-(trifluoromethyl)pyridin-2-yl]oxy}phenyl)ethyl]quinazolin-4-amine, (2.034) N-(2-cyclopentyl-5-fluorobenzyl)-N-cyclopropyl-3-(difluoromethyl)-5-fluoro-1-methyl-1H-pyrazole-4-carboxamide, (2.035) N-(2-tert-butyl-5-methylbenzyl)-N-cyclopropyl-3-(difluoromethyl)-5-fluoro-1-methyl-1H-pyrazole-4-carboxamide, (2.036) N-(2-tert-butylbenzyl)-N-cyclopropyl-3-(difluoromethyl)-5-fluoro-1-methyl-1H-pyrazole-4-carboxamide, (2.037) N-(5-chloro-2-ethylbenzyl)-N-cyclopropyl-3-(difluoromethyl)-5-fluoro-1-methyl-1H-pyrazole-4-carboxamide, (2.038) N-(5-chloro -2-isopropylbenzyl)-N-cyclopropyl-3-(difluoromethyl)-5-fluoro -1-methyl-1H-pyrazole-4-carboxamide, (2.039) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041) N-[1-(2,4-dichlorophenyl)-1-methoxypropan-2-yl]-3-(difluoromethyl)-1-methyl-1H-pyrazole-4-carboxamide, (2.042) N-[2-chloro-6-(trifluoromethyl)benzyl]-N-cyclopropyl-3-(difluoromethyl)-5-fluoro-1-methyl-1H-pyrazole-4-carboxamide, (2.043) N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045) N-cyclopropyl-3-(difluoromethyl)-5-fluoro-1-methyl-N-[5-methyl-2-(trifluoromethyl)benzyl]-1H-pyrazole-4-carboxamide, (2.046) N-cyclopropyl-3-(difluoromethyl)-5-fluoro-N-(2-fluoro-6-isopropylbenzyl)-1-methyl-1H-pyrazole-4-carboxamide, (2.047) N-cyclopropyl-3-(difluoromethyl)-5-fluoro-N-(2-isopropyl-5-methylbenzyl)-1-methyl-1H-pyrazole-4-carboxamide, (2.048) N-cyclopropyl-3-(difluoromethyl)-5-fluoro-N-(2-isopropylbenzyl)-1-methyl-1H-pyrazole-4-carbothioamide, (2.049) N-cyclopropyl-3-(difluoromethyl)-5-fluoro-N-(2-isopropylbenzyl)-1-methyl-1H-pyrazole-4-carboxamide, (2.050) N-cyclopropyl-3-(difluoromethyl)-5-fluoro -N-(5-fluoro-2-isopropylbenzyl)-1-methyl-1H-pyrazole-4-carboxamide, (2.051) N-cyclopropyl-3-(difluoromethyl)-N-(2-ethyl-4,5-dimethylbenzyl)-5-fluoro-1-methyl-1H-pyrazole-4-carboxamide, (2.052) N-cyclopropyl-3-(difluoromethyl)-N-(2-ethyl-5-fluorobenzyl)-5-fluoro-1-methyl-1H-pyrazole-4-carboxamide, (2.053) N-cyclopropyl-3-(difluoromethyl)-N-(2-ethyl-5-methylbenzyl)-5-fluoro-1-methyl-1H-pyrazole-4-carboxamide, (2.054) N-cyclopropyl-N-(2-cyclopropyl-5-fluorobenzyl)-3-(difluoromethyl)-5-fluoro-1-methyl-1H-pyrazole-4-carboxamide, (2.055) N-cyclopropyl-N-(2-cyclopropyl-5-methylbenzyl)-3-(difluoromethyl)-5-fluoro-1-methyl-1H-pyrazole-4-carboxamide, (2.056) N-cyclopropyl-N-(2-cyclopropylbenzyl)-3-(difluoromethyl)-5-fluoro-1-methyl-1H-pyrazole-4-carboxamide, (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027) N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate, (4.001) carbenclazim, (4.002) diethofencarb, (4.003) ethaboxam, (4.004) fluopicolide, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro -4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro -4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023) N-(2-bromo -6-fluorophenyl)-4-(2-chloro-4-fluorophenyl)-1,3-dimethyl-1H-pyrazol-5-amine, (4.024) N-(2-bromophenyl)-4-(2-chloro-4-fluorophenyl)-1,3-dimethyl-1H-pyrazol-5-amine, (4.025) N-(4-chloro-2,6-difluorophenyl)-4-(2-chloro-4-fluorophenyl)-1,3-dimethyl-1H-pyrazol-5-amine. (5.001) bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) metiram zinc, (5.017) oxine-copper, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile, (6.001) acibenzolar-S-methyl, (6.002) isotianil, (6.003) probenazole, (6.004) tiadinil, (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinolone, (8.001) silthiofam, (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one, (10.001) propamocarb, (10.002) propamocarb hydrochloride, (10.003) tolclofos-methyl, (11.001) tricyclazole, (11.002) 2,2,2-trifluoroethyl {3-methyl-1-[(4-methylbenzoyl)amino]butan-2-yl}carbamate, (12.001) benalaxyl, (12.002) benalaxyl-M (kiralaxyl), (12.003) metalaxyl, (12.004) metalaxyl-M (mefenoxam), (13.001) fludioxonil, (13.002) iprodione, (13.003) procymidone, (13.004) proquinazid, (13.005) quinoxyfen, (13.006) vinclozolin, (14.001) fluazinam, (14.002) meptyldinocap, (15.001) Abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) Oxathiapiprolin, (15.023) oxyfenthiin, (15.024) pentachlorophenol and salts, (15.025) phosphorous acid and its salts, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro -6-(prop-2-yn-1-yloxy)phenyl]-4,5-dihydro-1,2-oxazol-3-yl}-1,3-thiazol-2-yl)ethanone, (15.037) 2-[3,5-bis(difluoromethyl)-1H-pyrazol-1-yl]-1-[4-(4-{5-[2-fluoro -6-(prop-2-yn-1-yloxy)phenyl]-4,5-dihydro-1,2-oxazol-3-1,3-thiazol-2-yl)piperidin-1-yl]ethanone, (15.038) 2-[6-(3-fluoro -4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts, (15.046) 3-(4,4,5-trifluoro-3,3-dimethyl-3,4-dihydroisoquinolin-1-yl)quinoline, (15.047) 3-(4,4-difluoro -3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butanoic acid, (15.050) 5-amino-1,3,4-thiadiazole-2-thiol, (15.051) 5-chloro-N-phenyl-N-(prop-2-yn-1-yl)thiophene-2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, and (15.062) 5-fluoro-4-imino-3-methyl-1-[(4-methylphenyl)sulfonyl]-3,4-dihydropyrimidin-2(1H)-one.

Seed Treatment

The invention furthermore includes a method for treating seed.

A further aspect of the present invention relates in particular to seeds (dormant, primed, pregerminated or even with emerged roots and leaves) treated with at least one of the compounds of the formula (I). The inventive seeds are used in methods for protection of seeds and emerged plants from the seeds from phytopathogenic harmful fungi. In these methods, seed treated with at least one inventive active ingredient is used.

The compounds of the formula (I) are also suitable for the treatment of seeds and young seedlings. A large part of the damage to crop plants caused by harmful organisms is triggered by the infection of the seeds before sowing or after germination of the plant. This phase is particularly critical since the roots and shoots of the growing plant are particularly sensitive, and even small damage may result in the death of the plant. Accordingly, there is great interest in protecting the seed and the germinating plant by using appropriate compositions.

It is also desirable to optimize the amount of the active ingredient used so as to provide the best possible protection for the seeds, the germinating plants and emerged seedlings from attack by phytopathogenic fungi, but without damaging the plants themselves by the active ingredient used. In particular, methods for the treatment of seed should also take into consideration the intrinsic phenotypes of transgenic plants in order to achieve optimum protection of the seed and the germinating plant with a minimum of crop protection compositions being employed.

The present invention therefore also relates to a method for protecting seeds, germinating plants and emerged seedlings against attack by animal pests and/or phytopathogenic harmful microorganisms by treating the seeds with an inventive composition. The invention also relates to the use of the compositions according to the invention for treating seeds for protecting the seeds, the germinating plants and emerged seedlings against animal pests and/or phytopathogenic microorganisms. The invention further relates to seeds which has been treated with an inventive composition for protection from animal pests and/or phytopathogenic microorganisms.

One of the advantages of the present invention is that the treatment of the seeds with these compositions not only protects the seed itself, but also the resulting plants after emergence, from animal pests and/or phytopathogenic harmful microorganisms. In this way, the immediate treatment of the crop at the time of sowing or shortly thereafter protect plants as well as seed treatment in prior to sowing. It is likewise considered to be advantageous that the inventive active ingredients or compositions can be used especially also for transgenic seed, in which case the plant which grows from this seed is capable of expressing a protein which acts against pests, herbicidal damage or abiotic stress. The treatment of such seeds with the inventive active ingredients or compositions, for example an insecticidal protein, can result in control of certain pests. Surprisingly, a further synergistic effect can be observed in this case, which additionally increases the effectiveness for protection against attack by pests, microorganisms, weeds or abiotic stress.

The compounds of the formula (I) are suitable for protection of seed of any plant variety which is used in agriculture, in the greenhouse, in forests or in horticulture. More particularly, the seed is that of cereals (such as wheat, barley, lye, millet and oats), oilseed rape, maize, cotton, soybeen, rice, potatoes, sunflower, beans, coffee, beet (e.g. sugar beet and fodder beet), peanut, vegetables (such as tomato, cucumber, onions and lettuce), lawns and ornamental plants. Of particular significance is the treatment of the seed of wheat, soybean, oilseed rape, maize and rice.

As also described below, the treatment of transgenic seed with the inventive active ingredients or compositions is of particular significance. This refers to the seed of plants containing at least one heterologous gene which allows the expression of a polypeptide or protein, e.g. having insecticidal properties. These heterologous genes in transgenic seeds may originate, for example, from microorganisms of the species Bacillus, Rhizobium, Pseudomonas, Serratia, Trichoderma, Clavibacter, Glomus or Gliocladium. These heterologous genes preferably originates from Bacillus sp., in which case the gene product is effective against the European corn borer and/or the Western corn rootworm. Particularly preferably, the heterologous genes originate from Bacillus thuringiensis.

In the context of the present invention, the inventive composition is applied to seeds either alone or in a suitable formulation. Preferably, the seed is treated in a state in which it is sufficiently stable for no damage to occur in the course of treatment. In general, seeds can be treated at any time between harvest and some time after sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again, or seeds just after priming, or seeds stored in primed conditions or pre-germinated seeds, or seeds sown on nursery trays, tapes or paper.

When treating the seeds, it generally has to be ensured that the amount of the inventive composition applied to the seed and/or the amount of further additives is selected such that the germination of the seed is not impaired, or that the resulting plant is not damaged This must be ensured particularly in the case of active ingredients which can exhibit phytotoxic effects at certain application rates.

The compounds of the formula (I) can be applied directly, i.e. without containing any other components and without having been diluted. In general, it is preferable to apply the compositions to the seed in the form of a suitable formulation. Suitable formulations and methods for seed treatment are known to those skilled in the art. The compounds of the formula (I) can be converted to the customary formulations relevant to on-seed applications, such as solutions, emulsions, suspensions, powders, foams, slurries or combined with other coating compositions for seed, such as film forming materials, pelleting materials, fine iron or other metal powders, granules, coating material for inactivated seeds, and also ULV formulations.

These formulations are prepared in a known manner, by mixing the active ingredients or active ingredient combinations with customary additives, for example customary extenders and solvents or diluents, dyes, wetting agents, dispersants, emulsifiers, antifoams, preservatives, secondary thickeners, adhesives, gibberellins, and also water.

Useful dyes which may be present in the seed dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of active agrochemical ingredients. Usable with preference are alkylnaphthalenesulphonates, such as diisopropyl- or diisobutylnaphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Usable with preference are nonionic or anionic dispersants or mixtures of nonionic or anionic dispersants. Useful nonionic dispersants include especially ethylene oxide/propylene oxide block polymers, alkylphenol polyglycol ethers and tristryrylphenol polyglycol ether, and the phosphated or sulphated derivatives thereof. Suitable anionic dispersants are especially lignosulphonates, polyacrylic acid salts and arylsulphonate/formaldehyde condensates.

Antifoams which may be present in the seed dressing formulations usable in accordance with the invention are all foam-inhibiting substances conventionally used for the formulation of active agrochemical ingredients. Silicone antifoams and magnesium stearate can be used with preference.

Preservatives which may be present in the seed dressing formulations usable in accordance with the invention are all substances usable for such purposes in agrochemical compositions. Examples include dichlorophen and benzyl alcohol hemiformal.

Secondary thickeners which may be present in the seed dressing formulations usable in accordance with the invention are all substances usable for such purposes in agrochemical compositions. Preferred examples include cellulose derivatives, acrylic acid derivatives, xanthan, modified clays and finely divided silica.

Adhesives which may be present in the seed dressing formulations usable in accordance with the invention are all customary binders usable in seed dressing products. Preferred examples include polyvinylpyrrolidone, polyvinyl acetate, polyvinyl alcohol and tylose.

The formulations for on-seed applications usable in accordance with the invention can be used to treat a wide variety of different kinds of seed either directly or after prior dilution with water. For instance, the concentrates or the preparations obtainable therefrom by dilution with water can be used to dress the seed of cereals, such as wheat, barley, rye, oats, and triticale, and also seeds of maize, soybean, rice, oilseed rape, peas, beans, cotton, sunflowers, and beets, or else a wide variety of different vegetable seeds. The formulations usable in accordance with the invention, or the dilute preparations thereof, can also be used for seeds of transgenic plants. In this case, additional synergistic effects may also occur in interaction with the substances formed by expression.

For treatment of seeds with the formulations usable in accordance with the invention, or the preparations prepared therefrom by adding water, all mixing units usable customarily for on-seed applications are useful. Specifically, the procedure in on-seed applications is to place the seeds into a mixer, to add the particular desired amount of the formulations, either as such or after prior dilution with water, and to mix everything until all applied formulations are distributed homogeneously on the seeds. If appropriate, this is followed by a drying operation.

The application rate of the formulations usable in accordance with the invention can be varied within a relatively wide range. It is guided by the particular content of the active ingredients in the formulations and by the seeds. The application rates of each single active ingredient is generally between 0.001 and 15 g per kilogram of seed, preferably between 0.01 and 5 g per kilogram of seed.

Antimycotic Effects

In addition, the compounds of the formula (I) also have very good antimycotic effects. They have a very broad antimycotic activity spectrum, especially against dermatophytes and yeasts, moulds and diphasic fungi (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The enumeration of these fungi by no means constitutes a restriction of the mycotic spectrum covered, and is merely of illustrative character.

The compounds can be used also to control important fungal pathogens in fish and crustacea farming, e.g. saprolegnia diclina in trouts, saprolegnia parasitica in crayfish.

The compounds of the formula (I) can therefore be used both in medical and in non-medical applications.

The compounds of the formula (I) can be used as such, in the form of their formulations or the use forms prepared therefrom, such as ready-to-use solutions, suspensions, wettable powders, pastes, soluble powders, dusts and granules. Application is accomplished in a customary manner, for example by watering, spraying, atomizing, broadcasting, dusting, foaming, spreading-on and the like. It is also possible to deploy the active ingredients by the ultra-low volume method or to inject the active ingredient preparation/the active ingredient itself into the soil. It is also possible to treat the seed of the plants.

GMO

As already mentioned above, it is possible to treat all plants and their parts in accordance with the invention. In a preferred embodiment, wild plant species and plant cultivars, or those obtained by conventional biological breeding methods, such as crossing or protoplast fusion, and also parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts” or “parts of plants” or “plant parts” have been explained above. More preferably, plants of the plant cultivars which are commercially available or are in use are treated in accordance with the invention. Plant cultivars are understood to mean plants which have new properties (“traits”) and have been obtained by conventional breeding, by mutagenesis or by recombinant DNA techniques. They can be cultivars, varieties, bio- or genotypes.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference—RNAi—technology or microRNA—miRNA—technology). A heterologous gene that is located in the genome is also called a transgene. A transgene that is defined by its particular location in the plant genome is called a transformation or transgenic event.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content and composition for example cotton or starch, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stresses).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance.

Plants or plant cultivars (obtained by plant biotechnology methods such as genetic engineering) which may also be treated according to the invention show altered quantity, quality and/or storage-stability of the harvested product and/or altered properties of specific ingredients of the harvested product.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics.

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering.

Plants or plant cultivars (that can be obtained by plant biotechnology methods such as genetic engineering) which may also be treated according to the invention are plants, such as Tobacco plants, with altered post-translational protein modification patterns.

Application Rates

When using the compounds of the formula (I) as fungicides, the application rates can be varied within a relatively wide range, depending on the kind of application. The application rate of the inventive active ingredients is
in the case of treatment of plant parts, for example leaves: from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used);
in the case of seed treatment: from 0.1 to 200 g per 100 kg of seed, preferably from 1 to 150 g per 100 kg of seed, more preferably from 2.5 to 25 g per 100 kg of seed, even more preferably from 2.5 to 12.5 g per 100 kg of seed;
in the case of soil treatment: from 0.1 to 10 000 g/ha, preferably from 1 to 5000 g/ha.

These application rates are merely by way of example and are not limiting for the purposes of the invention.

### PREPARATION EXAMPLES

### Preparation of 5-chloro-1-[[2-[4-(4-chlorophenoxy)-2-(bifluoromethyl)phenyl]-1,3-dioxolan-2-yl]methyl]imidazole (I-04)

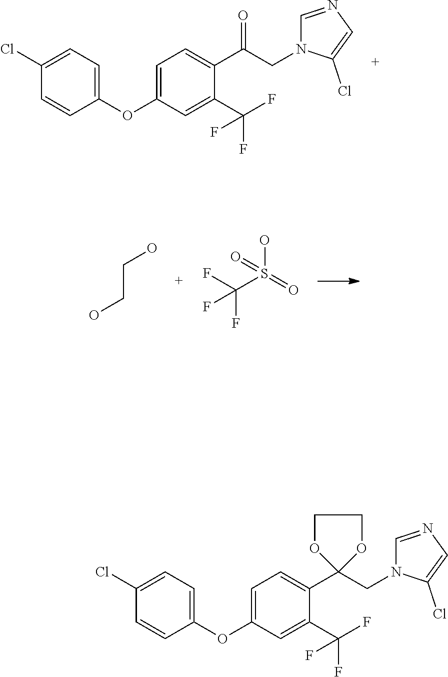

A mixture of 2-(5-chloroimidazol-1-yl)-1-[4-(4-chlorophenoxy)-2-(trifluoromethyl)phenyl]ethanone (0.43 g, 1.04 mmol), 1,2-ethanediol (1.2 mL, 20.7 mmol), triflic acid (0.46 mL, 5.2 mmol) in toluene (5.0 mL) was heated at 100° C. for 20 h, before the reaction was cooled to room temperature (rt; 21° C.) and quenched by addition of water. The mixture was extracted with dichloromethane, washed with brine, dried over MgSO₄, and concentrated. Preparative HPLC yielded 5-chloro-1-[[2-[4-(4-chlorophenoxy)-2-(trifluoromethyl)phenyl]-1,3-dioxolan-2-yl]methyl]imidazole (65.9 mg, 14% yield, 100% pure) as colorless oil.

MS (ESI): 459.2 ([M+H]⁺)

### Preparation of 6-(4-chlorophenoxy)-3-[2-[(5-fluoroimidazol-1-yl)methyl]-1,3-dioxolan-2-yl]-2-(trifluoromethyl)pyridine (I-18)

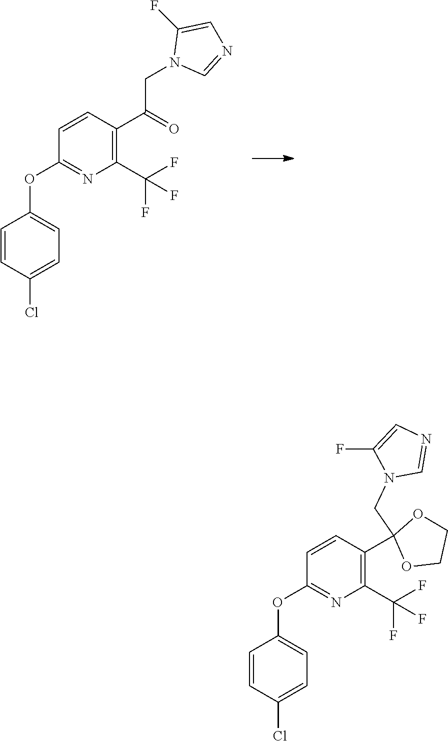 [SMILES: O=C(Cn1cncc1F)c1ccc(Oc2ccc(Cl)cc2)nc1C(F)(F)F ; Fc1cncn1CC1(c2ccc(Oc3ccc(Cl)cc3)nc2C(F)(F)F)OCCO1]

A mixture of 1-[6-(4-chlorophenoxy)-2-(trifluoromethyl)-3-pyridyl]-2-(5-fluoroimidazol-1-yl)ethanone (1.00 g, 2.50 mmol), 1,2-ethanediol (2.8 mL, 50.0 mmol), triflic acid (1.1 mL, 12.5 mmol) in toluene (6.0 mL) was heated at 110° C. for 60 h, before the reaction was cooled to rt and quenched by addition of water. The mixture was extracted with dichloromethane, washed with brine, dried over MgSO₄, and concentrated. Preparative HPLC yielded 6-(4-chlorophenoxy)-3-[2-[(5-fluoroimidazol-1-yl]methyl]-1,3-dioxolan-2-yl]-2-(trifluoromethyl)pyridine (246 mg, 21% yield, 96% pure) as colorless oil.

MS (ESI): 444.07 ([M+H]⁺)

### Preparation of 3-[2-[(5-fluoroimidazol-1-yl)methyl]-1,3-dioxolan-2-yl]-2-(trifluoromethyl)-6-[[6-(trifluoromethyl)-3-pyridyl]oxy]pyridine (I-19)

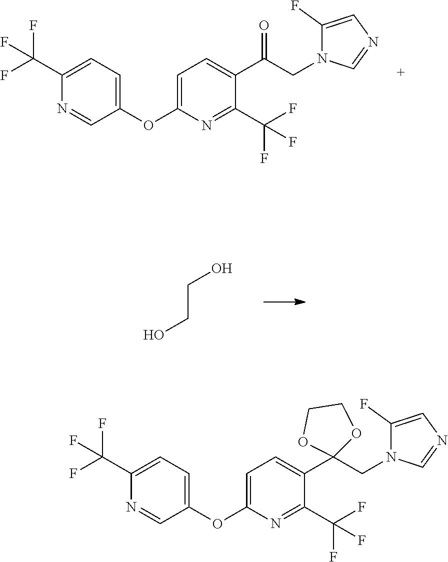

A mixture of 2-(5-fluoroimidazol-1-yl)-1-[2-(trifluoromethyl)-6-[[6-(trifluoromethyl)-3-pyridyl]oxy]-3-pyridyl]ethanone (1.00 g, 2.28 mmol), 1,2-ethanediol (2.8 g, 45.5 mmol), triflic acid (1.0 mL, 11.4 mmol) in toluene (12.0 mL) was heated at 110° C. for 20 h, before the reaction was cooled to rt and quenched by addition of water. The mixture was extracted with dichloromethane, washed with brine, filtered over ChemElut, and concentrated. Preparative HPLC yielded 3-[2-[(5-fluoroimidazol-1-yl)methyl]-1,3-dioxolan-2-yl]-2-(trifluoromethyl)-6-[[6-(trifluoromethyl)-3-pyridyl]oxy]pyridine (163 mg, 15% yield, 99% pure) as colorless oil.

MS (ESI): 479.09 ([M+H]⁺)

### Synthesis of 2-(5-fluoroimidazol-1-yl)-1-[2-(trifluoromethyl)-6-[[6-(trifluoromethyl)-3-pyridyl]oxy]-3-pyridyl]ethanone

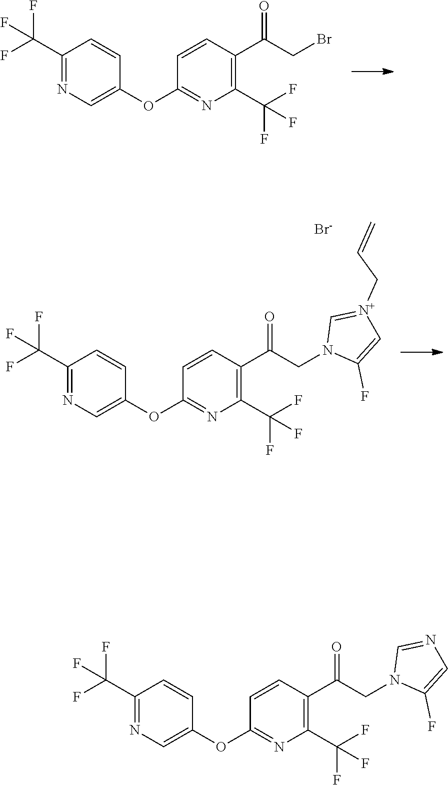 [SMILES: O=C(CBr)c1ccc(Oc2ccc(C(F)(F)F)nc2)nc1C(F)(F)F ; C=CC[n+]1cc(F)n(CC(=O)c2ccc(Oc3ccc(C(F)(F)F)nc3)nc2C(F)(F)F)c1 ; [Br-] ; O=C(Cn1cncc1F)c1ccc(Oc2ccc(C(F)(F)F)nc2)nc1C(F)(F)F]

Step 1: A solution of 2-bromo-1-[2-(trifluoromethyl)-6-[[6-(trifluoromethyl)-3-pyridyl]oxy]-3-pyridyl]ethanone (3.82 g, 8.90 mmol) and 1-allyl-4-fluoro-imidazole (1.23 g, 8.90 mmol) in acetonitrile (40.0 mL) was heated at 80° C. for 60 h, and then the solvent was evaporated. The remaining oil was treated with diisopropylether until precipitation occurred, the solid was filtered and washed with little cold diisopropylether, and dried to give 2-(3-allyl-5-fluoro-imidazol-3-ium-1-yl)-1-[2-(trifluoromethyl)-6-[[6-(trifluoromethyl)-3-pyridyl]oxyl-3-pyridyl]ethanone bromide (2.76 g, 55% yield, 99% pure) as a light brown solid.

MS (ESI): 475.10 ([M−Br]⁺)

Step 2: A solution of 2-(3-allyl-5-fluoro-imidazol-3-ium-1-yl)-1-[2-(trifluoromethyl)-6-1-[[6-(trifluoromethyl)-3-pyridyl]oxy]-3-pyridyl]ethanone bromide (2.76 g, 4.97 mmol), morpholine (0.52 mL, 5.95 mmol), tetrakis(triphenylphosphine)palladium(0) (114.9 mg, 0.10 mmol) in acetonitrile (40 mL) was stirred at rt for 4 h, before the mixture was quenched with water, filtered over ChemElut, washed with dichloromethane, and concentrated. Flash column chromatography (dichloromethane/methanol) yielded 2-(5-fluoroimidazol-1-yl)-1-[2-(trifluoromethyl)-6-1-[[6-(trifluoromethyl)-3-pyridyl]oxy]-3-pyridyl]ethanone (1.72 g, 79% yield, 99% pure) as a pale yellow solid.

MS (ESI): 435.06 ([M+H]⁺)

### Synthesis of 2-bromo-1-[2-(trifluoromethyl)-6-1-[[6-(trifluoromethyl)-3-pyridyl]oxy]-3-pyridyl]ethanone

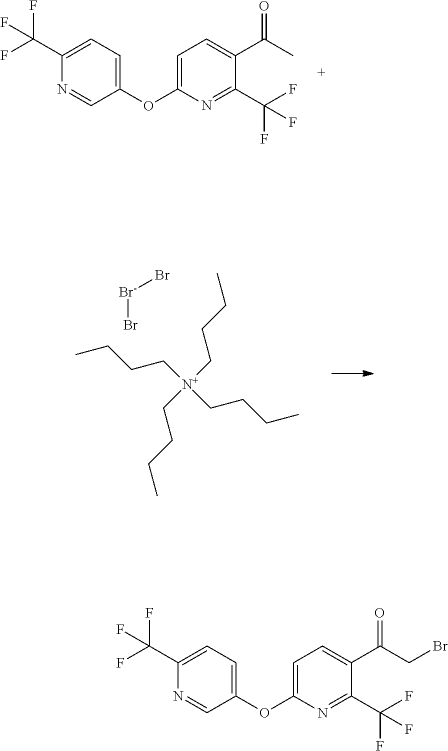

A solution of 1-[2-(trifluoromethyl)-6-1-[[6-(trifluoromethyl)-3-pyridyl]oxy]-3-pyridyl]ethanone (7.30 g, 20.8 mmol) and tetra-n-butylammonium perbromide (10.0 g, 20.8 mmol) in acetonitrile (200 mL) was stirred at rt for 20 h, then concentrated and purified via flash column chromatography (heptane/ethyl acetate) to give 2-bromo-1-[2-(trifluoromethyl)-6-1-[[6-(trifluoromethyl)-3-pyridyl]oxy]-3-pyridyl]ethanone (7.68 g, 66%, 77% pure) as a yellow solid.

MS (ESI): 428.96 ([M+H]⁺)

### Synthesis of 1-[2-(trifluoromethyl)-6-1-[[6-(trifluoromethyl)-3-pyridyl]oxy]-3-pyridyl]ethanone

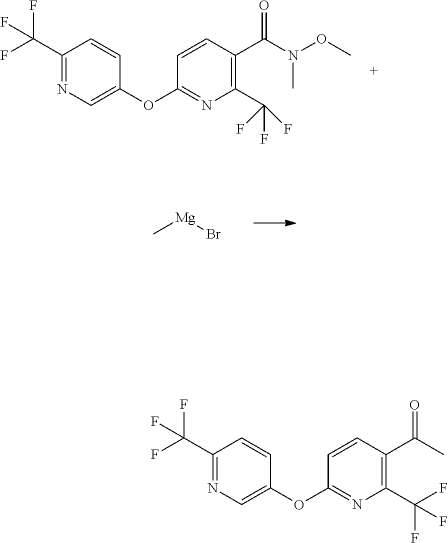

To a solution of N-methoxy-N-methyl-2-(trifluoromethyl)-6-[[6-(trifluoromethyl)-3-pyridyfloxylpyridine-3-carboxamide (12.9 g, 32.7 mmol) in THF (200 mL) at 0° C. was added a solution of methylmagnesium bromide (21.8 mL, 3 M in diethyl ether, 65.4 mmol) dropwise, and the reaction mixture was allowed to reach rt, where the mixture was stirred for another 3 h. LCMS analysis revealed that the reaction was not complete at that point, so another 3 mL of methylmagnesium bromide were added at rt, and the mixture was stirred for another 2 h at rt. The reaction was then quenched by addition of saturated aqueous ammonium chloride solution and water, the organic phase was separated, passed over a paper phase separation filter, and concentrated to give 1-[2-(trifluoromethyl)-6-[[6-(trifluoromethyl)-3-pyridyl]oxy]-3-pyridyllethanone (10.9 g, 91% yield, 95% pure) as a brown solid.

MS (ESI): 351.05 ([M+H]⁺)

### Synthesis of N-methoxy-N-methyl-2-(trifluoromethyl)-6-[[6-(trifluoromethyl)-3-pyridyl]oxy]pyridine-3-carboxamide

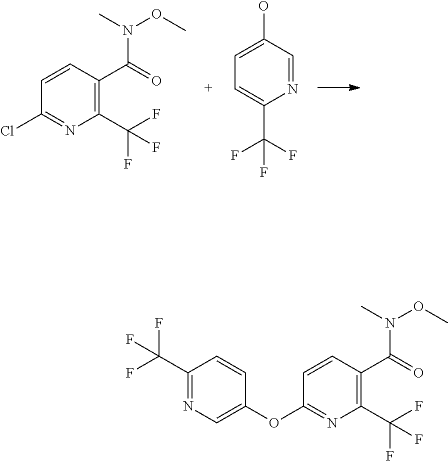 [SMILES: CON(C)C(=O)c1ccc(Cl)nc1C(F)(F)F ; Oc1ccc(C(F)(F)F)nc1 ; CON(C)C(=O)c1ccc(Oc2ccc(C(F)(F)F)nc2)nc1C(F)(F)F]

A solution of 6-chloro-N-methoxy-N-methyl-2-(trifluoromethyl)pyridine-3-carboxamide (10.0 g, 37.2 mmol), 6-trifluoromethyl-3-pyridinol (6.07 g, 37.2 mmol), potassium carbonate (12.9 g, 93.1 mmol), 1,2-bis(dimethylamino)ethane (0.86 g, 7.45 mmol) and CuI (0.71 g, 3.72 mmol) in dimethyl sulfoxide (50 mL) was heated at 100° C. for 12 h, then concentrated. The resulting oil was taken up in ethyl acetate and water, extracted with ethyl acetate, dried (Na₂SO₄), filtered, and concentrated to give N-methoxy-N-methyl-2-(trifluoromethyl)-6-[[6-(trifluoromethyl)-3-pyridyl]oxy]pyridine-3-carboxamide (12.9 g, 85% yield, 97% pure) as a brown oil. MS (ESI): 396.07 ([M+H]⁺)

### Preparation of 5-chloro-1-[[2-[2-chloro-4-(4-chlorophenoxy)phenyl]-1,3-dioxolan-2-yl]methyl]-imidazole (I-01)

### Preparation of 2-chloro-1-[2-chloro-4-(4-chlorophenoxy)phenyl]ethanone

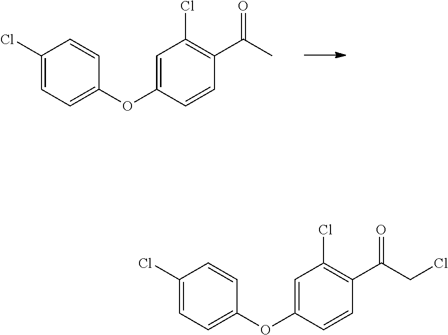 [SMILES: CC(=O)c1ccc(Oc2ccc(Cl)cc2)cc1Cl ; O=C(CCl)c1ccc(Oc2ccc(Cl)cc2)cc1Cl]

To a stirred solution of 1-[2-chloro-4-(4-chlorophenoxy)phenyl]ethanone (1.00 g; 3.55 mmol) and methanol (456 mg; 14.2 mmol; 4.0 eq) in dry dichloromethane (12 mL) at room temperature was added dropwise a solution of sulfuryl chloride (672 mg; 4.98 mmol; 1.4 eq) in dichloromethane (2 mL). The resulting mixture was stirred at room temperature for 40 h, then refluxed for 2 h. Additional sulfuryl chloride (240 mg; 1.78 mmol; 0.5 eq) was added via syringe, and the mixture further stirred at room temperature for 22 h. Thereafter the reaction mixture was diluted with saturated aqueous sodium bicarbonate, extracted with dichloromethane, the combined organic layers were dried and concentrated to dryness in vacuo. The oily residue was purified by chromatography over silica gel, eluted with a mixture of n-heptane/ethyl acetate (100:0 to 95:5). After evaporation of the solvent 553 mg (49%) of 2-chloro-1-[2-chloro-4-(4-chlorophenoxy)phenyl]ethanone were obtained as pale yellow oil.

MS (ESI): 315.0 ([M+H]⁺)

### Preparation of 1-[2-chloro-4-(4-chlorophenoxy)phenyl]-2-(5-chloroimidazol-1-yl)ethanone

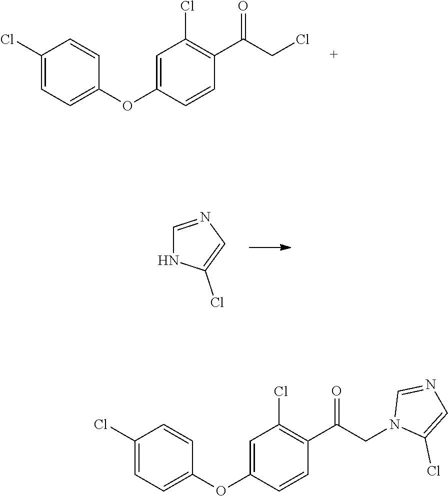 [SMILES: O=C(CCl)c1ccc(Oc2ccc(Cl)cc2)cc1Cl ; Clc1cnc[nH]1 ; O=C(Cn1cncc1Cl)c1ccc(Oc2ccc(Cl)cc2)cc1Cl]

A solution of 2-chloro-1-[2-chloro-4-(4-chlorophenoxy)phenyl]ethanone (550 mg; 1.74 mmol) and 4-chloroimidazole (214 mg; 2.09 mmol; 1.20 eq) in dry acetonitrile (5.0 mL) was stirred at 80° C. for 20 h, then at 130° C. for 1 h under microwave irradiation. Thereafter the reaction mixture was allowed to cool down to room temperature, diluted with water, the organic layer was washed with saturated aqueous sodium bicarbonate, dried (MgSO₄) and concentrated to dryness in vacuo. The oily residue was purified by chromatography over silica gel, eluted with a mixture of n-heptane/ethyl acetate (100:0 to 0:100). After evaporation of the solvents, a second purification by preparative HPLC was performed. Evaporation of the solvents in vacuo afforded 141 mg (21%) of 1-[2-chloro-4-(4-chlorophenoxy)phenyl]-2-(5-chloroimidazol-1-yl)ethanone as a colourless solid.

MS (ESI): 381.0 ([M+H]⁺)

### Preparation of 5-chloro-1-[[2-[2-chloro-4-(4-chlorophenoxy)phenyl]-1,3-dioxolan-2-yl]methyl]imidazole (I-01)

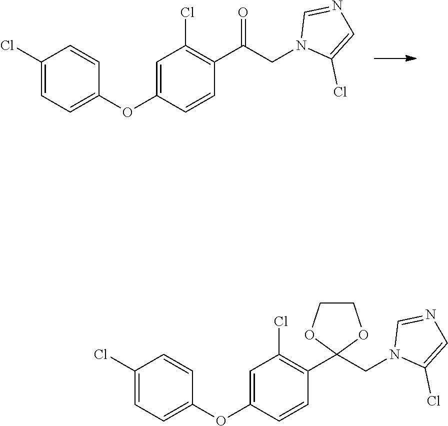 [SMILES: O=C(Cn1cncc1Cl)c1ccc(Oc2ccc(Cl)cc2)cc1Cl ; Clc1ccc(Oc2ccc(C3(Cn4cncc4Cl)OCCO3)c(Cl)c2)cc1]

Under an atmosphere of argon, trifluoromethanesulfonic acid (0.23 mL; 393 mg; 2.62 mmol; 5.0 eq) was added dropwise at 0-5° C. (ice/brine bath) to a solution of 1-[2-chloro-4-(4-chlorophenoxy)phenyl]-2-(5-chloroimidazol-1-yl)ethanone (220 mg; 0.52 mmol) and ethylene glycol (0.58 mL; 650 mg; 10.4 mmol; 20.0 eq) in anhydrous toluene (3.0 mL). The resulting mixture was allowed to warm up to room temperature, then refluxed for 20 h. Thereafter the reaction mixture was allowed to cool down to room temperature, diluted with ethyl acetate, washed with saturated aqueous sodium bicarbonate, the organic layers were dried (MgSO₄) and concentrated to dryness in vacuo. The oily residue was purified by chromatography over silica gel, eluted with a mixture of n-heptane/ethyl acetate (100:0 to 80:20). After evaporation of the solvents, a second purification by preparative HPLC was performed. Evaporation of the solvents in vacuo afforded 128 mg (57%) of 5-chloro-1-[[2-[2-chloro-4-(4-chlorophenoxy)phenyl]-1,3-dioxolan-2-yl]methyl]imidazole as a colourless solid.

MS (ESI): 425.0 ([M+H]⁺)

### Preparation of 2-(5-bromoimidazol-1-yl)-1-[2-chloro-4-(4-chlorophenoxy)phenyl]ethanone (I-02)

### Preparation of 2-bromo-1-[2-chloro-4-(4-chlorophenoxy)phenyl]ethanone

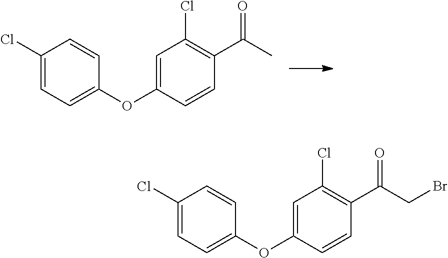 [SMILES: CC(=O)c1ccc(Oc2ccc(Cl)cc2)cc1Cl ; O=C(CBr)c1ccc(Oc2ccc(Cl)cc2)cc1Cl]

To a stirred solution of 1-[2-chloro-4-(4-chlorophenoxy)phenyl]ethanone (10.0 g; 35.5 mmol) in dry tetrahydrofuran (250 mL), cooled to 0-5° C. (ice/brine bath), was added phenyltrimethylammonium tribromide (14.0 g; 37.3 mmol; 1.05 eq). The resulting mixture was stirred at room temperature for 20 h. Thereafter the reaction mixture was diluted with water, extracted with ethyl acetate, the combined organic layers were washed with saturated aqueous sodium bicarbonate, saturated aqueous sodium thiosulfate, then finally with water. The organic layer was dried (MgSO₄) and concentrated to dryness in vacuo. The oily residue was purified by chromatography over silica gel, eluted with a mixture of n-heptane/ethyl acetate (100:0 to 95:5). After evaporation of the solvent 11.2 g (75%) of 2-bromo-1-[2-chloro-4-(4-chlorophenoxy)phenyl]ethanone were obtained as pale yellow oil.

MS (ESI): 358.9 ([M+H]⁺)

### Preparation of 1-allyl-4-bromo-imidazole

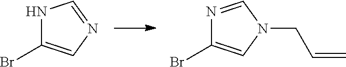

To a solution of 4-bromo-1H-imidazole (1.00 g, 6.80 mol) in dry DMF (10 mL) was added solid potassium carbonate (1.13 g; 8.16 mmol; 1.20 eq). After stirring 40 min at room temperature, a solution of allyl bromide (882 mg; 7.14 mmol; 1.05 eq) in dry DMF (10 mL) was added dropwise. The resulting mixture was stirred at room temperature for 6 h. Thereafter the reaction mixture was diluted with water, extracted with ethyl acetate, the combined organic layers were dried (MgSO₄) and concentrated in vacuo. The oily residue was purified by chromatography over silica gel, eluted with a mixture of n-heptane/ethyl acetate (100:0 to 80:20). After evaporation of the solvent 892 mg (63%) of a mixture comprising 90 mol % 1-allyl-4-bromo-imidazole and 10 mol % of its 5-bromo regioisomer were obtained as pale yellow oil.

MS (EI): 187.0 ([M]⁺)

### Preparation of 2-(5-bromoimidazol-1-yl)-1-[2-chloro-4-(4-chlorophenoxy)phenyl]ethanone

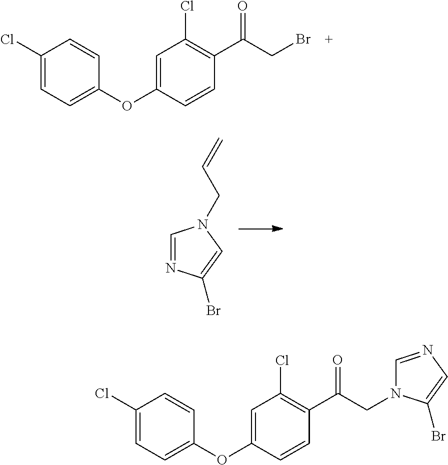

A solution of 1-allyl-4-bromo-imidazole (750 mg; 3.80 mmol) and 2-bromo-1-[2-chloro-4-(4-chlorophenoxy)phenyl]ethanone (1.51 g; 4.19 mmol; 1.10 eq) in dry acetonitrile (10 mL) was refluxed under stirring for 20 h. Thereafter the reaction mixture was allowed to cool down to room temperature, and concentrated to dryness in vacuo. After dilution in a minimum of dichloromethane, pentane was carefully added until a precipitation took place. After stirring for 30 min at room temperature, the solid was filtered off and dried in vacuo to provide 1.92 g (92%) of intermediate imidazolium bromide as a colourless solid.

MS (ESI): 465.0 ([M−Br]⁺)

To a solution of this imidazolium bromide (1.90 g; 3.47 mmol) in degassed anhydrous dichloromethane (100 mL) was added, under an atmosphere of argon, solid tetrakis(triphenylphosphine)palladium(0) (120 mg; 100 μmol; 0.03 eq) followed by morpholine (0.36 mL; 363 mg; 4.16 mmol; 1.20 eq). The resulting mixture was stirred under an atmosphere of argon at room temperature for 18 h. Thereafter the reaction mixture was filtered over a plug of celite and the filtrate was concentrated to dryness in vacuo. The oily residue was purified by chromatography over silica gel, eluted with a mixture of n-heptane/ethyl acetate (100:0 to 0:100). After evaporation of the solvents, a second purification by preparative HPLC was performed. Evaporation of the solvents in vacuo afforded 228 mg (15%) of 2-(5-bromoimidazol-1-yl)-1-[2-chloro-4-(4-chlorophenoxy)phenyl]ethanone as a colourless solid.

MS (ESI): 424.9 ([M+H]⁺)

### Preparation of 5-bromo-1-[[2-[2-chloro-4-(4-chlorophenoxy)phenyl]-1,3-dioxolan-2-yl]methyl]imidazole (I-02)

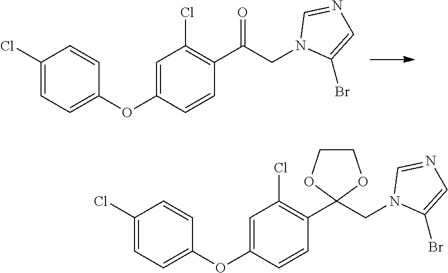 [SMILES: O=C(Cn1cncc1Br)c1ccc(Oc2ccc(Cl)cc2)cc1Cl ; Clc1ccc(Oc2ccc(C3(Cn4cncc4Br)OCCO3)c(Cl)c2)cc1]

Under an atmosphere of argon, trifluoromethanesulfonic acid (555 mg; 3.69 mmol; 5.0 eq) was added dropwise at 0-5° C. (ice/brine bath) to a solution of 2-(5-bromoimidazol-1-yl)-1-[2-chloro-4-(4-chlorophenoxy)phenyl]ethanone (350 mg; 0.73 mmol) and ethylene glycol (1.84 g; 29.5 mmol; 40.0 eq) in anhydrous toluene (3.0 mL). The resulting mixture was allowed to warm up to room temperature, then refluxed for 20 h. Thereafter the reaction mixture was allowed to cool down to room temperature, diluted with ethyl acetate, washed with aqueous sodium hydroxide (1 M), the combined organic layers were dried (MgSO₄) and concentrated to dryness in vacuo. The oily residue was purified by chromatography over silica gel, eluted with a mixture of dichloromethane/methanol (100:0 to 90:10). After evaporation of the solvents, a second purification by preparative HPLC was performed. Evaporation of the solvents in vacuo afforded 211 mg (61%) of 5-bromo-1-[[2-[2-chloro-4-(4-chlorophenoxy)phenyl]-1,3-dioxolan-2-yl]methyl]imidazole as a colourless solid.

MS (ESI): 469.0 ([M+H]⁺)

### Preparation of 1-[[2-[2-chloro-4-(4-chlorophenoxy)phenyl]-4-methyl-1,3-dioxolan-2-yl]methyl]-5-fluoro-imidazole (I-12)

### Preparation of 1-allyl-4-fluoro-imidazole

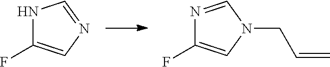

To a solution of 5-fluoro-1H-imidazole (9.40 g, 109 mmol) in anhydrous THF (250 mL), cooled to 0-5° C. (ice/brine bath), was added solid sodium hydride (60% wt dispersion in mineral oil; 5.24 g; 131.0 mmol; 1.20 eq). After stirring for 20 min at 0-5° C., a solution of allyl bromide (10.1 mL; 14.2 g; 114.6 mmol; 1.05 eq) in anhydrous THF (50 mL) was added dropwise. The resulting mixture was stirred at room temperature for 20 h. Thereafter the reaction mixture was carefully diluted with water, extracted with ethyl acetate, the combined organic layers were dried (MgSO₄) and concentrated in vacuo. The oily residue was purified by chromatography over silica gel, eluted with a mixture of n-heptane/ethyl acetate (100:0 to 50:50). After evaporation of the solvent 11.4 g (78%) of 1-allyl-4-fluoro-imidazole were obtained as pale yellow oil.

MS (EI): 127.0 ([M]⁺)

### Preparation of 1-[2-chloro-4-(4-chlorophenoxy)phenyl]-2-(5-fluoroimidazol-1-yl)ethanone

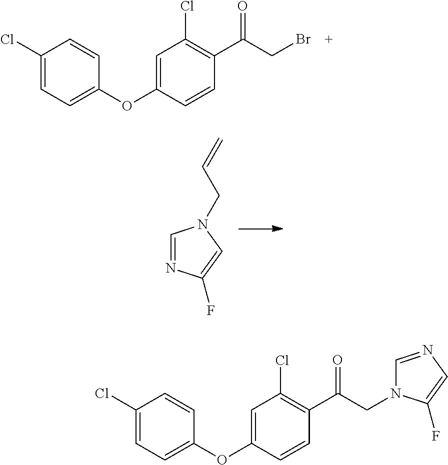

A solution of 1-allyl-4-fluoro-imidazole (800 mg; 6.34 mmol) and 2-bromo-1-[2-chloro-4-(4-chlorophenoxy)phenyl]ethanone (2.79 g; 6.98 mmol; 1.10 eq) in dry acetonitrile (10 mL) was refluxed under stirring for 20 h. Thereafter the reaction mixture was allowed to cool down to room temperature and concentrated to dryness in vacuo. After dilution in a minimum of dichloromethane, pentane was carefully added until a precipitation took place. After stirring for 30 min at room temperature, the solid was filtered off and dried in vacuo to provide 2.63 g (85%) of intermediate imidazolium bromide as a colourless solid.

MS (ESI): 405.0 ([M−Br]⁺)

To a solution of this imidazolium bromide (2.60 g; 5.34 mmol) in degassed anhydrous dichloromethane (100 mL) was added, under an atmosphere of argon, solid tetrakis(triphenylphosphine)palladium(0) (124 mg; 107 μmol; 0.02 eq) followed by morpholine (0.56 mL; 559 mg; 6.42 mmol; 1.20 eq). The resulting mixture was stirred under an atmosphere of argon at room temperature for 18 h. Thereafter the reaction mixture was filtered over a plug of celite. The filtrate was diluted with water, extracted with dichloromethane, the organic layer was dried (MgSO₄) and concentrated to dryness in vacuo. The residue was purified by chromatography over silica gel, eluted with a mixture of n-heptane/ethyl acetate (100:0 to 0:100). Evaporation of the solvents in vacuo afforded 1.80 g (92%) of 1-[2-chloro-4-(4-chlorophenoxy)phenyl]-2-(5-fluoroimidazol-1-yl)ethanone as an off-white solid.

MS (ESI): 365.0 ([M+H]⁺)

### Preparation of 1-[[2-[2-chloro-4-(4-chlorophenoxy)phenyl]-4-methyl-1,3-dioxolan-2-yl]methyl]-5-fluoro-imidazole (I-12)

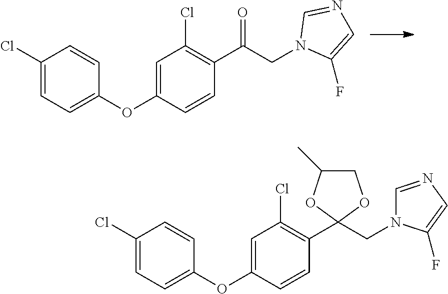 [SMILES: O=C(Cn1cncc1F)c1ccc(Oc2ccc(Cl)cc2)cc1Cl ; CC1COC(Cn2cncc2F)(c2ccc(Oc3ccc(Cl)cc3)cc2Cl)O1]

Under an atmosphere of argon, trifluoromethanesulfonic acid (485 μL; 822 mg; 5.47 mmol; 5.0 eq) was added dropwise at 0-5° C. (ice/brine bath) to a solution of 1-[2-chloro-4-(4-chlorophenoxy)phenyl]-2-(5-fluoroimidazol-1-yl)ethanone (400 mg; 1.09 mmol) and (2S)-propane-1,2-diol (3.33 g; 43.8 mmol; 40.0 eq) in anhydrous toluene (3.0 mL). The resulting mixture was allowed to warm up to room temperature, then refluxed for 20 h. Thereafter the reaction mixture was allowed to cool down to room temperature, diluted with ethyl acetate, washed with saturated aqueous sodium bicarbonate, the combined organic layers were dried (MgSO₄) and concentrated to dryness in vacuo. The residue was purified by chromatography over silica gel, eluted with a mixture of dichloromethane/methanol (100:0 to 90:10). Evaporation of the solvents in vacuo afforded 271 mg (54%) of 1-[[2-[2-chloro-4-(4-chlorophenoxy)phenyl]-4-methyl-1,3-dioxolan-2-yl]methyl]-5-fluoro-imidazole (approx. 59:41 mixture of diastereoisomers) as a colourless solid.

MS (ESI): 423.1 ([M+H]⁺)

### Preparation of 1-[(2-{2-chloro-4-[4-(trifluoromethyl)phenoxy]phenyl}-1,3-dioxolan-2-yl)methyl]-5-ethynyl-1H-imidazole (I-30)

### Step 1: Preparation of 1-[(2-{2-chloro-4-[4-(trifluoromethyl)phenoxy]phenyl}-1,3-dioxolan-2-yl)methyl]-5-[(trimethylsilyl)ethynyl]-1H-imidazole

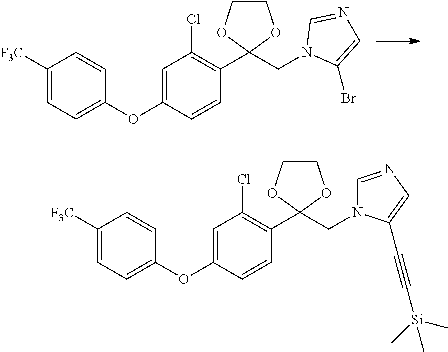 [SMILES: FC(F)(F)c1ccc(Oc2ccc(C3(Cn4cncc4Br)OCCO3)c(Cl)c2)cc1 ; C[Si](C)(C)C#Cc1cncn1CC1(c2ccc(Oc3ccc(C(F)(F)F)cc3)cc2Cl)OCCO1]

A solution of 5-bromo-1-[(2-{2-chloro-4-[4-(trifluoromethyl)phenoxy]phenyl}-1,3-dioxolan-2-yl)methyl]-1H-imidazole (556 mg; 1.08 mmol) and trimethylamine (528 μL; 383 mg; 3.78 mmol; 3.5 eq) in thy THF (10 mL) was filled in a reactor, degassed and covered by an argon atmosphere. To this solution were rapidly added ethynyl(trimethyl)silane (420 μL; 297 mg; 3.02 mmol; 2.8 eq), copper(I) iodide (41 mg; 0.21 mmol; 0.20 eq) and tetrakis(triphenylphosphine)palladium(0) (125 mg; 0.10 mmol; 0.1 eq). The reactor was sealed and the content stirred at 50° C. for 24 h. Thereafter the reaction mixture was diluted with water and dichloromethane, filtered over ChemElut, the organic layer was concentrated to dryness in vacuo. The brown oily residue was purified by chromatography over silica gel, eluted with a mixture of dichloromethane/ethyl acetate (100:0 to 70:30). Evaporation of the solvents in vacuo yielded 201 mg (36%) of the desired compound.

MS (ESI): 522.1 ([M+H]⁺)

### Step 2: Preparation of 1-[(2-{2-chloro-4-[4-(trifluoromethyl)phenoxy]phenyl}-1,3-dioxolan-2-yl)methyl]-5-ethynyl-1H-imidazole (I-30)

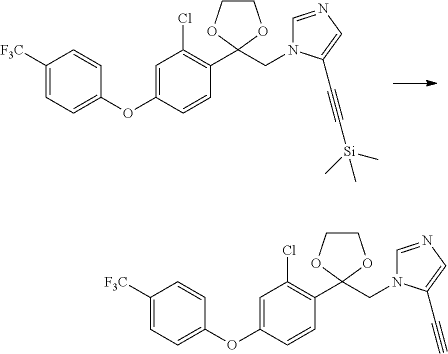 [SMILES: C[Si](C)(C)C#Cc1cncn1CC1(c2ccc(Oc3ccc(C(F)(F)F)cc3)cc2Cl)OCCO1 ; C#Cc1cncn1CC1(c2ccc(Oc3ccc(C(F)(F)F)cc3)cc2Cl)OCCO1]

To a solution of 1-[(2-{2-chloro-4-[4-(trifluoromethyl)phenoxy]phenyl}-1,3-dioxolan-2-yl]methyl]-5-[(trimethylsilyl)ethynyl]-1H-imidazole (190 mg; 0.36 mmol) in anhydrous THF (5 mL) was added tetra-n-butylammonium fluoride (1 M solution in anhydrous THF, 547 μL; 0.55 mmol; 1.5 eq). The resulting mixture was stirred at room temperature for 2 h. Thereafter the reaction mixture was diluted with EtOAc and washed with water. The aqueous layer was extracted with EtOAc, the combined organic layers were dried over MgSO₄ and concentrated to dryness in vacuo to afford 153 mg (93%) of the target compound as a colourless solid.

MS (ESI): 449.1 ([M+H]⁺)

### Preparation of 1-{[2-{2-chloro-4-[4-(trifluoromethyl)phenoxy]phenyl}-4-methyl-1,3-dioxolan-2-yl]methyl}-1H-imidazole-5-carbonitrile (I-32)

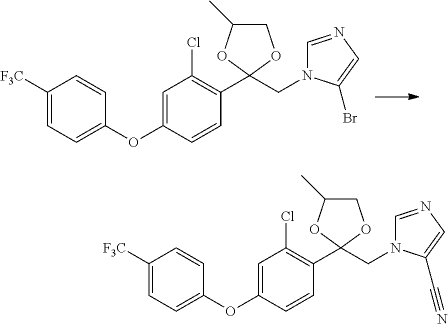 [SMILES: CC1COC(Cn2cncc2Br)(c2ccc(Oc3ccc(C(F)(F)F)cc3)cc2Cl)O1 ; CC1COC(Cn2cncc2C#N)(c2ccc(Oc3ccc(C(F)(F)F)cc3)cc2Cl)O1]

To a solution of 5-bromo-1-{[2-{2-chloro-4-[4-(trifluoromethyl)phenoxy]phenyl}-4-methyl-1,3-dioxolan-2-yl]methyl}-1H-imidazole (200 mg; 0.38 mmol), zinc(II) cyanide (45.4 mg; 0.38 mmol; 1.0 eq) and palladium(n-cinnamyl) chloride dimer (10.0 mg, 0.01 mmol, 0.05 eq) in degassed N,N-dimethylacetamide (2.0 mL) under an atmosphere of argon, was added diisopropylethylamine (0.067 mL; 0.38 mmol; 1.0 eq) via syringe. The reactor was sealed and the resulting mixture was stirred at 120° C. for 20 h. Thereafter the reaction mixture was diluted with brine, extracted with EtOAc, the organic layer was washed with saturated aqueous potassium carbonate and then finally with water. The aqueous layer was extracted with EtOAc, the combined organic layers were dried over MgSO₄ and concentrated to dryness in vacuo. The residue was purified by chromatography over silica gel, eluted with a mixture of heptane/ethyl acetate (100:0 to 50:50). Evaporation of the solvents in vacuo afforded 132 mg (70%) of the target compound (approx. 65:35 mixture of diastereoisomers) as a colourless solid.

MS (ESI): 464.5 ([M+H]⁺)

The following tables illustrate in a non-limiting manner examples of compounds according to the invention.

Table 1
Compounds according to formula (I)

| | | | | (I) |
|---|---|---|---|---|
| 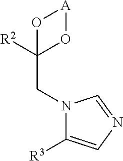 | | | | |
| Ex N° | A | R² | R³ | LogP |
| I-01 | —CH₂CH₂— | 2-chloro-4-(4-chlorophenoxy)phenyl | chloro | 3.29^{[a]} |
| I-02 | —CH₂CH₂— | 2-chloro-4-(4-chlorophenoxy)phenyl | bromo | 3.35^{[a]} |
| I-03 | —CH₂CH₂— | 2-chloro-4-(4-iodophenoxy)phenyl | chloro | 3.82^{[a]} |
| I-04 | —CH₂CH₂— | 4-(4-chlorophenoxy)-2-(trifluoromethyl)phenyl | chloro | 3.75^{[a]} |
| I-05 | —CH₂CH₂— | 4-[(6-chloropyridin-3-yl)oxy]-2-(trifluoromethyl)phenyl | chloro | 2.58^{[a]} |
| I-06 | —CH₂CH₂— | 4-(4-bromophenoxy)-2-(trifluoromethyl)phenyl | chloro | 3.77^{[a]} |
| I-07 | —CH₂CH₂— | 4-(4-bromophenoxy)-2-chlorophenyl | chloro | 3.53^{[a]} |
| I-08 | —CH₂CH₂— | 2-chloro-4-(4-iodophenoxy)phenyl | bromo | 3.76^{[a]} |
| I-09 | —CH₂CH(CH₃)— | 2-chloro-4-(4-chlorophenoxy)phenyl | chloro | 3.64^{[a]} |
| I-10 | —CH₂CH₂— | 2-chloro-4-(4-chlorophenoxy)phenyl | trifluoromethyl | 4.64^{[a]} |
| I-11 | —CH₂CH₂— | 2-chloro-4-(4-chlorophenoxy)phenyl | fluoro | 2.88^{[a]} |
| I-12 | —CH₂CH(CH₃)— | 2-chloro-4-(4-chlorophenoxy)phenyl | fluoro | 3.06 + 303.02^{[a]} |
| I-13 | —CH₂CH₂— | 2-chloro-4-[(6-chloropyridin-3-yl)oxy]phenyl | chloro | 2.42^{[a]} |
| I-14 | —CH₂CH₂— | 2-chloro-4-[(5-chloropyridin-2-yl)oxy]phenyl | chloro | 2.66^{[a]} |
| I-15 | —CH₂CH₂— | 4-(4-chlorophenoxy)-2-(trifluoromethyl)phenyl | fluoro | 3.06^{[a]} |
| I-16 | —CH₂CH₂— | 6-(4-bromophenoxy)-2-(trifluoromethyl)pyridin-3-yl | fluoro | 2.78^{[a]} |
| I-17 | —CH₂CH₂— | 6-(4-chlorophenoxy)-2-fluoropyridin-3-yl | fluoro | 2.34^{[a]} |
| I-18 | —CH₂CH₂— | 6-(4-chlorophenoxy)-2-(trifluoromethyl)pyridin-3-yl | fluoro | 2.42^{[a]} |
| I-19 | —CH₂CH₂— | 2-(trifluoromethyl)-6-{[6-(trifluoromethyl)pyridin-3- | fluoro | 2.53^{[a]} |
| | | yl]oxy}pyridin-3-yl | | |
| I-20 | —CH₂CH₂— | 2-(trifluoromethyl)-6-{[6-(bromo)pyridin-3- | fluoro | 2.17^{[a]} |
| | | yl]oxy}pyridin-3-yl | | |
| I-21 | —CH₂CH₂— | 4-(4-bromophenoxy)-2-chlorophenyl | bromo | 3.48^{[a]} |
| I-22 | —CH₂CH₂— | 2-chloro-4-(4-chlorophenoxy)phenyl | ethynyl | 2.94^{[a]} |
| I-23(*) | —CH₂CH(CH₃)— | 2-chloro-4-(4-chlorophenoxy)phenyl | bromo | 3.68^{[a]} |
| I-24 | —CH₂CH₂— | 2-chloro-4-[4-(2-trimethylsilylethyn-1- | bromo | 4.98 ^{[a]} |
| | | yl)phenoxy]phenyl | | |
| I-25 | —CH₂CH₂— | 2-chloro-4-(4-ethynylphenoxy)phenyl | ethynyl | 2.76 ^{[a]} |
| I-26(*) | —CH₂CH(CH₃)— | 2-chloro-4-(4-chlorophenoxy)phenyl | ethynyl | 3.15 ^{[a]} |
| I-27 | —CH₂CH₂— | 4-(4-chlorophenoxy)-2-(trifluoromethyl)phenyl | bromo | 3.58 ^{[a]} |
| I-28 | —CH₂CH₂— | 4-(4-chlorophenoxy)-2-(trifluoromethyl)phenyl | ethynyl | 3.13 ^{[a]} |
| I-29 | —CH₂CH₂— | 2-chloro-4-[4-(trifluoromethyl)phenoxy]phenyl | bromo | 3.44 ^{[a]} |
| I-30 | —CH₂CH₂— | 2-chloro-4-[4-(trifluoromethyl)phenoxy]phenyl | ethynyl | 3.04 ^{[a]} |
| I-31(*) | —CH₂CH(CH₃)— | 2-chloro-4-[4-(trifluoromethyl)phenoxy]phenyl | bromo | 3.85 ^{[a]} |
| I-32(*) | —CH₂CH(CH₃) | 2-chloro-4-[4-(trifluoromethyl)phenoxy]phenyl | cyano | 4.08 ^{[a]} |
| I-33 | —CH₂CH₂— | 2-chloro-4-(4-chlorophenoxy)phenyl | cyano | 3.78 ^{[a]} |
| I-34 | —CH₂CH₂— | 2-chloro-4-[4-(trifluoromethyl)phenoxy]phenyl | cyano | 3.92 ^{[a]} |
| I-35 | —CH₂CH₂— | 4-(4-chlorophenoxy)-2-(trifluoromethyl)phenyl | cyano | 4.03 ^{[a]} |

(*)only one pair of enantiomers

TABLE 2
Compounds according to formula (I-RP)

| | | | | (I-RP) |
|---|---|---|---|---|
| 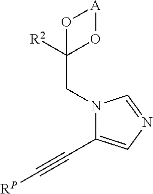 | | | | |
| Ex N° | A | R² | R^{P} | LogP |
| I-RP-01 | —CH₂CH₂— | 2-chloro-4-(4-chlorophenoxy)phenyl | trimethylsilyl | 4.54 ^{[a]} |
| I-RP-02 | —CH₂CH₂— | 2-chloro-4-[4-(2-trimethylsilylethyn-1-yl)phenoxy]phenyl | trimethylsilyl | 6.17 ^{[a]} |
| I-RP-03 (*) | —CH₂CH(CH₃)— | 2-chloro-4-(4-chlorophenoxy)phenyl | trimethylsilyl | 4.90 ^{[a]} |
| I-RP-04 | —CH₂CH₂— | 4-(4-chlorophenoxy)-2-(trifluoromethyl)phenyl | trimethylsilyl | 4.67 ^{[a]} |
| I-RP-05 | —CH₂CH₂— | 2-chloro-4-[4-(trifluoromethyl)phenoxy]phenyl | trimethylsilyl | 4.59 ^{[a]} |

(*) only one pair of enantiomers

Log P Values:

Measurement of Log P values was performed according to EEC directive 79/831 Annex V.A8 by HPLC (High Performance Liquid Chromatography) on reversed phase columns with the following methods:
^{[a]} Log P value is determined by measurement of LC-UV, in an acidic range, with 0.1% formic acid in water and acetonitrile as eluent (linear gradient from 10% acetonitrile to 95% acetonitrile).
^{[b]} Log P value is determined by measurement of LC-UV, in a neutral range, with 0.001 molar ammonium acetate solution in water and acetonitrile as eluent (linear gradient from 10% acetonitrile to 95% acetonitrile).
^{[c]} Log P value is determined by measurement of LC-UV, in an acidic range, with 0.1% phosphoric acid and acetonitrile as eluent (linear gradient from 10% acetonitrile to 95% acetonitrile).

If more than one Log P value is available within the same method, all the values are given and separated by “+”.

Calibration was done with straight-chain alkan-2-ones (with 3 to 16 carbon atoms) with known Log P values (measurement of Log P values using retention times with linear interpolation between successive alkanones).

Lambda-max-values were determined using UV-spectra from 200 nm to 400 nm and the peak values of the chromatographic signals.

NMR-Peak Lists

1H-NMR data of selected examples are written in form of 1H-NMR-peak lists. To each signal peak are listed the δ-value in ppm and the signal intensity in round brackets. Between the δ-value—signal intensity pairs are semicolons as delimiters.

The peak list of an example has therefore the form:

δ₁ (intensity₁); δ₂ (intensity₂); . . . ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contains therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical 1H-NMR interpretation.

Further details of NMR-data description with peak lists you find in the publication “Citation of NMR Peaklist Data within Patent Applications” of the Research Disclosure Database Number 564025.

I-01: ¹H-NMR (499.9 MHz, CDCl₃):
δ = 7.6053 (6.0); 7.5774 (4.6); 7.5600 (4.9); 7.3582 (0.8); 7.3517 (6.6); 7.3478 (2.8); 7.3340 (7.4); 7.3275 (1.1);
7.2641 (3.2); 7.0418 (4.6); 7.0370 (5.0); 6.9941 (1.0); 6.9876 (7.3); 6.9838 (3.0); 6.9699 (6.8); 6.9634 (1.0);
6.8959 (6.0); 6.8947 (6.0); 6.8533 (2.7); 6.8484 (2.7); 6.8359 (2.6); 6.8310 (2.6); 4.4692 (16.0); 3.8310 (1.7);
3.8168 (6.0); 3.8092 (3.1); 3.8032 (3.4); 3.7840 (0.9); 3.7603 (0.9); 3.7411 (3.2); 3.7352 (2.9); 3.7276 (6.2);
3.7133 (1.9); 1.7419 (2.6); −0.0002 (3.6)
I-02: ¹H-NMR (300.2 MHz, CDCl₃):
δ = 7.7120 (6.8); 7.6114 (4.8); 7.5825 (5.2); 7.3890 (0.6); 7.3780 (5.6); 7.3721 (4.1); 7.3556 (2.1); 7.3490 (7.2);
7.3430 (3.5); 7.3001 (1.2); 7.0673 (4.7); 7.0592 (5.4); 7.0284 (0.8); 7.0175 (7.3); 6.9949 (2.2); 6.9852 (7.9);
6.9825 (8.5); 6.8870 (2.8); 6.8790 (2.8); 6.8581 (2.6); 6.8501 (2.6); 4.5096 (16.0); 3.8612 (1.4); 3.8380 (4.9);
3.8309 (3.9); 3.8238 (3.2); 3.8151 (3.7); 3.7849 (2.6); 3.7552 (3.3); 3.7475 (3.0); 3.7404 (3.3); 3.7299 (5.4);
3.7089 (1.4); 0.0238 (0.9)
I-03: ¹H-NMR (300.2 MHz, CDCl₃):
δ = 7.7351 (0.8); 7.7249 (7.9); 7.7177 (2.5); 7.7024 (2.6); 7.6952 (8.5); 7.6851 (1.0); 7.6457 (5.0); 7.6427 (4.9);
7.6211 (5.3); 7.5922 (5.7); 7.2997 (5.4); 7.0869 (5.1); 7.0788 (5.6); 6.9331 (5.2); 6.9299 (5.1); 6.9044 (3.4);
6.8961 (3.1); 6.8755 (3.2); 6.8670 (3.6); 6.8563 (8.5); 6.8491 (2.7); 6.8338 (2.5); 6.8266 (7.8); 6.8165 (0.9);
5.3342 (0.9); 4.5044 (16.0); 3.8795 (1.4); 3.8584 (3.9); 3.8536 (3.0); 3.8480 (2.8); 3.8410 (2.8); 3.8332 (3.8);
3.8088 (1.8); 3.8041 (1.8); 3.7799 (3.9); 3.7720 (2.8); 3.7649 (2.9); 3.7593 (3.1); 3.7548 (4.0); 3.7333 (1.6);
1.7755 (2.8); 0.0343 (5.6)
I-04: ¹H-NMR (300.2 MHz, CDCl₃):
δ = 7.6817 (4.1); 7.6556 (3.2); 7.6456 (0.9); 7.6268 (3.4); 7.4184 (4.7); 7.4135 (10.0); 7.3913 (3.1); 7.3837 (9.5);
7.3726 (1.2); 7.2999 (125.5); 7.1103 (2.4); 7.1017 (2.2); 7.0813 (2.1); 7.0728 (2.0); 7.0462 (1.1); 7.0353 (9.3);
7.0279 (2.8); 7.0130 (2.6); 7.0055 (7.9); 6.9945 (0.9); 6.9490 (0.8); 6.9232 (4.2); 5.3396 (12.6); 4.3473 (16.0);
3.8630 (0.6); 3.8370 (2.0); 3.8304 (2.6); 3.8249 (3.5); 3.8164 (9.2); 3.8041 (9.3); 3.7956 (3.9); 3.7903 (2.8);
3.7834 (2.4); 3.7578 (0.7); 1.7755 (0.6); 1.6461 (9.3); 1.2924 (2.0); 0.9192 (0.6); 0.2338 (0.4); 0.1197 (1.3);
0.1076 (41.9); 0.0953 (2.0); 0.0492 (3.6); 0.0383 (138.4); 0.0274 (6.3); −0.0439 (0.4); −0.0882 (0.4); −0.0981
(0.4); −0.1605 (0.7)
I-05: ¹H-NMR (300.2 MHz, CDCl₃):
δ = 13.1077 (0.4); 8.2390 (3.5); 8.2353 (3.6); 8.2307 (3.5); 8.2271 (3.4); 7.6863 (6.3); 7.6565 (3.7); 7.6457 (1.1);
7.6041 (0.4); 7.4543 (4.3); 7.4459 (4.5); 7.4221 (1.2); 7.3968 (8.0); 7.3930 (13.3); 7.3845 (6.5); 7.3638 (0.9);
7.3559 (1.2); 7.2998 (177.2); 7.2522 (0.4); 7.1441 (2.2); 7.1355 (2.1); 7.1154 (2.2); 7.1070 (1.8); 6.9488 (1.0);
6.9226 (3.6); 5.3395 (5.4); 4.3790 (0.4); 4.3566 (16.0); 3.8843 (1.0); 3.8605 (3.5); 3.8540 (2.8); 3.8485 (3.5);
3.8422 (4.3); 3.8370 (7.5); 3.8183 (7.4); 3.8131 (4.1); 3.8069 (3.2); 3.8014 (3.0); 3.7949 (3.5); 3.7713 (1.3);
3.6271 (0.4); 3.6060 (0.4); 3.5084 (0.4); 1.8259 (0.5); 1.6945 (6.6); 1.5609 (0.6); 1.2945 (2.0); 0.9193 (0.4);
0.2346 (0.7); 0.1082 (2.4); 0.0499 (6.2); 0.0390 (202.6); 0.0281 (8.5); −0.1598 (0.7)
I-06: ¹H-NMR (300.2 MHz, CDCl₃):
δ = 7.6782 (5.2); 7.6573 (3.4); 7.6456 (1.3); 7.6281 (3.7); 7.5697 (1.0); 7.5590 (8.3); 7.5517 (2.9); 7.5364 (2.8);
7.5292 (9.0); 7.5187 (1.2); 7.4525 (0.6); 7.4416 (0.6); 7.4228 (4.2); 7.4147 (4.6); 7.3461 (0.9); 7.2998 (217.8);
7.1161 (2.2); 7.1074 (2.1); 7.0868 (1.9); 7.0771 (1.9); 6.9803 (9.0); 6.9731 (2.8); 6.9577 (2.8); 6.9505 (8.8);
6.9401 (1.0); 6.9197 (5.0); 4.3479 (16.0); 3.8640 (0.8); 3.8380 (2.3); 3.8321 (3.0); 3.8258 (3.8); 3.8177 (9.0);
3.8053 (9.2); 3.7849 (2.5); 3.7569 (0.7); 3.5531 (0.5); 1.5911 (61.6); 1.5135 (0.8); 1.3497 (1.0); 1.2920 (1.9);
0.9211 (0.7); 0.2344 (1.1); 0.1202 (1.7); 0.1081 (44.7); 0.0960 (1.7); 0.0647 (0.6); 0.0499 (7.4); 0.0389 (245.0);
0.0281 (10.7); −0.1594 (1.1); −1.0249 (0.6); −3.1475 (0.5)
I-07: ¹H-NMR (300.2 MHz, CDCl₃):
δ = 7.6396 (4.8); 7.6177 (5.2); 7.5888 (5.5); 7.5435 (0.7); 7.5326 (7.1); 7.5255 (2.3); 7.5102 (2.3); 7.5030 (7.8);
7.4921 (0.8); 7.2997 (1.7); 7.0789 (4.9); 7.0707 (5.4); 6.9807 (0.8); 6.9699 (8.0); 6.9627 (2.5); 6.9474 (2.4);
6.9402 (7.4); 6.9287 (5.4); 6.9263 (5.0); 6.8966 (3.1); 6.8883 (2.9); 6.8677 (2.9); 6.8593 (2.7); 4.5006 (16.0);
3.8747 (1.4); 3.8538 (4.0); 3.8490 (3.0); 3.8432 (2.8); 3.8362 (2.7); 3.8284 (3.7); 3.8044 (1.8); 3.7994 (1.8);
3.7755 (3.7); 3.7676 (2.7); 3.7606 (2.8); 3.7549 (3.0); 3.7500 (4.0); 3.7290 (1.4); 2.0357 (0.7); 0.0301 (1.9)
I-08: ¹H-NMR (300.2 MHz, CDCl₃):
δ = 7.7332 (1.2); 7.7232 (13.1); 7.7161 (3.0); 7.7069 (0.6); 7.7007 (2.6); 7.6935 (8.4); 7.6834 (1.0); 7.6210 (5.2);
7.5921 (5.7); 7.2997 (4.6); 7.0856 (5.1); 7.0774 (5.6); 6.9963 (5.7); 6.9931 (5.7); 6.9043 (3.4); 6.8960 (3.1);
6.8754 (3.2); 6.8670 (3.6); 6.8560 (8.5); 6.8489 (2.7); 6.8335 (2.5); 6.8264 (7.8); 6.8162 (0.9); 4.5201 (16.0);
3.8732 (1.5); 3.8517 (4.4); 3.8477 (3.2); 3.8420 (2.9); 3.8350 (2.8); 3.8269 (3.8); 3.7974 (2.8); 3.7679 (3.8);
3.7597 (2.8); 3.7527 (3.0); 3.7470 (3.4); 3.7431 (4.5); 3.7215 (1.7); 2.0401 (1.6); 1.8201 (0.6); 0.0339 (3.8)
I-09: ¹H-NMR (499.9 MHz, CDCl₃):
δ = 7.6319 (6.6); 7.6266 (4.9); 7.6089 (4.1); 7.5972 (6.5); 7.5910 (4.6); 7.5799 (6.6); 7.3513 (10.0); 7.3364 (8.6);
7.3338 (10.9); 7.2636 (10.4); 7.0348 (5.8); 7.0302 (9.3); 7.0260 (4.7); 7.0013 (1.0); 6.9947 (6.7); 6.9877 (10.0);
6.9824 (4.4); 6.9771 (6.4); 6.9700 (9.5); 6.9637 (1.8); 6.9068 (7.0); 6.8945 (4.6); 6.8591 (3.6); 6.8542 (3.7);
6.8501 (2.8); 6.8448 (2.8); 6.8420 (3.9); 6.8369 (3.7); 6.8327 (2.7); 6.8277 (2.5); 5.2983 (6.6); 4.5030 (3.6);
4.4735 (7.1); 4.4399 (4.6); 4.4117 (6.6); 4.3901 (4.6); 4.3823 (3.9); 4.3608 (2.3); 4.1127 (0.3); 4.1005 (1.3);
4.0873 (2.2); 4.0741 (2.6); 4.0617 (1.6); 4.0495 (0.5); 3.9700 (1.6); 3.9588 (2.0); 3.9540 (1.9); 3.9427 (2.0);
3.9231 (3.0); 3.9084 (3.8); 3.8959 (2.8); 3.8197 (0.8); 3.8076 (1.2); 3.8036 (1.1); 3.7957 (1.0); 3.7914 (1.4);
3.7795 (0.9); 3.7676 (0.3); 3.2973 (1.7); 3.2809 (3.3); 3.2647 (1.6); 3.0544 (2.7); 3.0393 (5.2); 3.0242 (2.7);
1.7250 (2.8); 1.2552 (0.7); 1.2429 (0.4); 1.1653 (9.8); 1.1531 (10.1); 1.1203 (15.6); 1.1082 (16.0); 1.0366 (0.4);
0.0709 (0.4); −0.0002 (8.2)
I-10: ¹H-NMR (300.2 MHz, CDCl₃):
δ = 7.8015 (5.6); 7.7994 (5.6); 7.6635 (5.1); 7.6345 (5.5); 7.4344 (4.3); 7.4081 (0.7); 7.3970 (6.8); 7.3899 (2.3);
7.3747 (2.4); 7.3674 (8.0); 7.3564 (0.9); 7.2997 (2.6); 7.0772 (4.9); 7.0690 (5.4); 7.0446 (0.9); 7.0336 (8.3);
7.0263 (2.5); 7.0112 (2.2); 7.0040 (6.9); 6.9929 (0.7); 6.9163 (3.1); 6.9080 (2.8); 6.8874 (2.8); 6.8791 (2.6);
4.5958 (16.0); 3.8724 (1.6); 3.8502 (4.9); 3.8417 (3.0); 3.8346 (2.7); 3.8259 (3.6); 3.7948 (1.4); 3.7889 (1.4);
3.7579 (3.6); 3.7491 (2.8); 3.7420 (3.0); 3.7334 (4.9); 3.7113 (1.6); 1.7777 (2.1); 0.0339 (1.8)
I-11: ¹H-NMR (400.0 MHz, d₆-DMSO):
δ = 7.5668 (7.0); 7.5450 (7.8); 7.5101 (1.2); 7.5015 (11.5); 7.4962 (3.9); 7.4847 (4.0); 7.4793 (13.1); 7.4707 (1.3);
7.2405 (4.5); 7.1629 (8.3); 7.1568 (8.8); 7.1450 (1.4); 7.1365 (12.9); 7.1310 (4.1); 7.1196 (3.7); 7.1142 (11.5);
7.1056 (1.1); 6.9762 (4.4); 6.9700 (4.1); 6.9545 (4.2); 6.9482 (4.0); 6.5064 (2.0); 6.4883 (2.0); 4.3958 (16.0);
3.9022 (5.6); 3.7854 (1.6); 3.7685 (4.9); 3.7646 (4.4); 3.7599 (4.6); 3.7549 (4.8); 3.7506 (6.3); 3.7454 (4.2);
3.7327 (4.1); 3.7276 (6.2); 3.7233 (4.8); 3.7182 (4.4); 3.7134 (4.4); 3.7096 (4.9); 3.6925 (1.6); 3.3246 (192.6);
2.6753 (1.1); 2.6708 (1.5); 2.6664 (1.2); 2.5062 (210.4); 2.5018 (271.8); 2.4973 (198.1); 2.3330 (1.1); 2.3284
(1.5); 2.3241 (1.1); 1.2353 (0.4); 0.0079 (0.6); −0.0002 (16.6); −0.0084 (0.6)
I-12: ¹H-NMR (499.9 MHz, CDCl₃):
δ = 7.6369 (3.5); 7.6195 (3.7); 7.6056 (5.6); 7.5883 (5.8); 7.3541 (6.5); 7.3512 (10.0); 7.3474 (3.8); 7.3367 (8.7);
7.3336 (10.3); 7.3273 (1.5); 7.2612 (15.4); 7.2335 (7.4); 7.1963 (4.7); 7.0363 (6.3); 7.0318 (9.3); 7.0276 (4.8);
7.0010 (0.9); 6.9944 (6.7); 6.9872 (9.8); 6.9826 (4.3); 6.9768 (6.3); 6.9695 (8.9); 6.9630 (1.2); 6.8604 (3.3);
6.8554 (3.4); 6.8519 (2.6); 6.8467 (2.5); 6.8431 (3.5); 6.8381 (3.4); 6.8346 (2.5); 6.8295 (2.1); 6.5026 (3.8);
6.4877 (6.0); 6.4728 (2.4); 5.2967 (0.6); 4.4006 (3.5); 4.3711 (6.3); 4.3610 (2.1); 4.3317 (4.3); 4.3019 (6.0);
4.2870 (4.4); 4.2725 (3.4); 4.2577 (2.0); 4.1279 (0.3); 4.1244 (0.4); 4.1181 (0.7); 4.1140 (0.5); 4.1062 (1.4);
4.0941 (2.4); 4.0793 (2.3); 4.0670 (1.5); 4.0547 (0.6); 4.0392 (0.4); 4.0285 (0.4); 3.9994 (1.6); 3.9882 (1.9);
3.9833 (1.8); 3.9721 (1.9); 3.9473 (0.6); 3.9373 (3.0); 3.9249 (3.4); 3.9224 (3.6); 3.9101 (2.8); 3.8543 (0.8);
3.8424 (1.2); 3.8378 (1.0); 3.8305 (0.9); 3.8259 (1.3); 3.8140 (0.8); 3.6999 (0.3); 3.6764 (0.4); 3.6696 (0.4);
3.6333 (0.4); 3.6202 (0.4); 3.3046 (1.8); 3.2882 (3.2); 3.2718 (1.6); 3.0802 (2.8); 3.0651 (5.3); 3.0499 (2.6);
2.1031 (3.9); 2.0912 (2.0); 2.0852 (1.0); 2.0796 (4.8); 2.0580 (0.6); 2.0416 (1.0); 1.6509 (1.0); 1.2720 (0.3);
1.2577 (0.7); 1.2485 (1.9); 1.2435 (0.7); 1.2356 (1.9); 1.2203 (2.3); 1.2075 (2.3); 1.1933 (1.3); 1.1891 (1.4);
1.1804 (1.6); 1.1759 (1.9); 1.1703 (10.2); 1.1582 (10.1); 1.1432 (15.9); 1.1311 (16.0); 1.1228 (2.0); 1.1135 (0.8);
1.1055 (0.6); 1.1011 (0.5); −0.0002 (16.4)
I-13: ¹H-NMR (300.2 MHz, CDCl₃):
δ = 8.2363 (2.8); 8.2314 (4.2); 8.2248 (2.7); 7.6586 (5.1); 7.6506 (6.3); 7.6298 (5.0); 7.5136 (0.4); 7.3819 (10.7);
7.3772 (8.2); 7.2995 (10.8); 7.1255 (4.3); 7.1173 (4.6); 6.9362 (6.2); 6.9336 (5.8); 6.9264 (3.2); 6.9180 (2.7);
6.8973 (2.6); 6.8890 (2.4); 4.5100 (16.0); 3.8909 (1.2); 3.8692 (3.8); 3.8643 (3.2); 3.8586 (3.1); 3.8516 (3.0);
3.8446 (3.8); 3.8287 (2.0); 3.8173 (2.0); 3.8014 (3.8); 3.7942 (3.0); 3.7872 (3.1); 3.7814 (3.4); 3.7769 (4.0);
3.7548 (1.2); 2.0825 (0.9); 1.6456 (8.5); 1.2964 (0.8); 0.0474 (0.4); 0.0367 (11.9); 0.0257 (0.5)
I-14: ¹H-NMR (300.2 MHz, CDCl₃):
δ = 8.1900 (4.0); 8.1812 (4.1); 7.7594 (2.6); 7.7505 (2.5); 7.7303 (3.2); 7.7236 (6.3); 7.6953 (5.2); 7.6747 (5.6);
7.2993 (14.6); 7.2841 (4.6); 7.2762 (4.9); 7.0951 (2.9); 7.0872 (2.6); 7.0664 (2.6); 7.0584 (2.4); 6.9997 (4.5);
6.9707 (4.3); 6.9518 (5.6); 5.3379 (7.9); 4.5285 (16.0); 4.4990 (0.4); 4.1715 (0.4); 4.1480 (0.4); 3.8933 (1.6);
3.8706 (5.2); 3.8635 (3.0); 3.8562 (2.5); 3.8470 (3.3); 3.8156 (1.0); 3.7964 (1.0); 3.7651 (3.4); 3.7558 (2.7);
3.7485 (3.2); 3.7423 (5.3); 3.7186 (1.6); 2.0835 (1.6); 1.6300 (8.5); 1.3210 (0.5); 1.2971 (1.4); 1.2736 (0.5);
0.0478 (0.6); 0.0372 (14.5)
I-15: ¹H-NMR (300.2 MHz, CDCl₃):
δ = 7.7295 (3.2); 7.7004 (3.6); 7.4262 (4.7); 7.4179 (12.4); 7.4106 (3.0); 7.3955 (2.8); 7.3881 (9.3); 7.3771 (1.0);
7.3665 (0.5); 7.2997 (42.2); 7.1300 (2.3); 7.1214 (2.2); 7.1011 (2.2); 7.0924 (2.0); 7.0532 (1.0); 7.0422 (9.4);
7.0348 (2.8); 7.0198 (2.5); 7.0125 (7.8); 7.0014 (0.7); 6.5505 (1.8); 6.5257 (1.7); 5.3393 (6.9); 4.2325 (16.0);
3.8607 (0.7); 3.8339 (2.1); 3.8278 (2.9); 3.8222 (4.0); 3.8143 (9.7); 3.8029 (9.8); 3.7951 (3.9); 3.7895 (2.8);
3.7833 (2.1); 3.7567 (0.6); 1.2923 (0.5); 0.1201 (0.4); 0.1079 (10.0); 0.0958 (0.4); 0.0493 (1.5); 0.0385 (43.1);
0.0276 (1.6)
I-16: ¹H-NMR (300.2 MHz, CDCl₃):
δ = 8.0669 (3.7); 8.0382 (3.9); 7.5911 (0.8); 7.5805 (8.2); 7.5735 (2.6); 7.5580 (2.7); 7.5509 (9.3); 7.5403 (1.0);
7.2998 (46.5); 7.2761 (6.0); 7.2732 (6.0); 7.1667 (1.0); 7.1560 (9.4); 7.1490 (2.9); 7.1335 (2.5); 7.1264 (8.0);
7.1158 (0.8); 7.0894 (4.6); 7.0606 (4.4); 6.5448 (2.9); 6.5418 (3.0); 6.5193 (3.1); 6.5163 (3.0); 4.2532 (16.0);
3.8696 (1.0); 3.8456 (3.2); 3.8391 (2.8); 3.8333 (3.4); 3.8267 (4.4); 3.8221 (8.2); 3.8048 (7.9); 3.8003 (4.5);
3.7937 (3.4); 3.7879 (2.8); 3.7814 (3.3); 3.7573 (1.0); 2.0471 (1.0); 1.6590 (3.5); 0.1074 (4.4); 0.0489 (1.4);
0.0380 (45.2); 0.0270 (1.6)
I-17: ¹H-NMR (300.2 MHz, CDCl₃):
δ = 8.7741 (1.1); 8.7455 (1.2); 8.2122 (1.3); 7.9743 (2.9); 7.9468 (4.0); 7.9422 (3.8); 7.9149 (3.0); 7.7389 (8.5);
7.7348 (10.6); 7.7299 (6.8); 7.7120 (13.7); 7.7070 (13.0); 7.6990 (10.3); 7.6948 (11.4); 7.6899 (7.2); 7.6720
(13.3); 7.6670 (11.7); 7.6162 (3.2); 7.6113 (3.5); 7.6062 (2.9); 7.5863 (13.3); 7.5725 (7.4); 7.5674 (9.8); 7.5624
(8.8); 7.5575 (4.7); 7.5293 (11.5); 7.5246 (11.0); 7.5222 (11.7); 7.5197 (12.1); 7.5151 (8.8); 7.5093 (11.4);
7.5044 (14.6); 7.4994 (14.3); 7.4949 (13.7); 7.4814 (6.8); 7.4762 (7.1); 7.4715 (6.3); 7.4674 (4.4); 7.4526 (1.6);
7.4454 (2.8); 7.4393 (2.2); 7.4284 (9.2); 7.4215 (4.5); 7.4058 (4.7); 7.3987 (10.8); 7.3882 (2.1); 7.3718 (1.5);
7.3424 (0.6); 7.3002 (11.8); 7.2964 (2.8); 7.2061 (2.0); 7.1765 (1.7); 7.1520 (2.0); 7.1414 (10.8); 7.1343 (5.0);
7.1250 (3.2); 7.1187 (4.5); 7.1117 (9.0); 7.1007 (1.9); 7.0966 (1.9); 6.8084 (4.5); 6.8049 (4.2); 6.7812 (4.6);
6.7776 (4.2); 6.6598 (3.4); 6.6354 (3.4); 5.3319 (1.3); 4.7777 (0.6); 4.7220 (1.2); 4.6548 (1.4); 4.5987 (1.7);
4.5716 (1.7); 4.3539 (16.0); 4.1649 (0.3); 4.1444 (0.8); 4.1282 (1.0); 4.1099 (0.8); 4.0731 (1.6); 4.0568 (1.0);
3.9272 (1.7); 3.9052 (5.3); 3.9003 (5.0); 3.8940 (4.8); 3.8872 (4.7); 3.8803 (5.3); 3.8647 (3.0); 3.8533 (3.0);
3.8377 (5.1); 3.8308 (4.7); 3.8238 (4.7); 3.8173 (5.1); 3.8126 (5.4); 3.7906 (1.7); 1.2890 (0.5); 0.1065 (2.3);
0.0445 (0.9); 0.0340 (11.4); 0.0302 (2.9)
I-18: ¹H-NMR (300.2 MHz, d₆-DMSO):
δ = 8.1102 (4.9); 8.0812 (5.4); 7.5801 (0.4); 7.5662 (1.2); 7.5549 (11.0); 7.5476 (3.7); 7.5326 (4.0); 7.5252 (13.8);
7.5140 (1.4); 7.3850 (6.2); 7.3561 (5.9); 7.3193 (1.8); 7.3082 (14.1); 7.3007 (4.5); 7.2924 (9.4); 7.2889 (9.9);
7.2785 (11.1); 7.2672 (1.1); 6.5489 (4.1); 6.5458 (4.1); 6.5232 (4.2); 6.5200 (4.0); 5.7768 (0.5); 5.5701 (0.4);
4.3325 (16.0); 3.7856 (1.3); 3.7619 (4.0); 3.7555 (3.6); 3.7494 (4.2); 3.7380 (8.7); 3.7188 (8.4); 3.7077 (4.1);
3.7015 (3.6); 3.6954 (4.0); 3.6716 (1.3); 3.3422 (21.9); 2.5332 (2.6); 2.5273 (5.7); 2.5212 (7.8); 2.5151 (5.7);
2.5092 (2.7); 0.0191 (5.5)
I-19: ¹H-NMR (300.2 MHz, CDCl₃):
δ = 8.7212 (3.5); 8.7129 (3.6); 8.1579 (3.8); 8.1291 (4.0); 7.9310 (1.4); 7.9224 (1.4); 7.9022 (2.4); 7.8937 (2.5);
7.8223 (5.5); 7.7936 (3.2); 7.3017 (14.7); 7.2790 (6.5); 7.2759 (6.4); 7.2677 (5.0); 7.2389 (4.4); 6.5474 (3.2);
6.5444 (3.1); 6.5219 (3.2); 6.5189 (3.0); 5.0721 (0.4); 4.2677 (16.0); 3.8909 (1.1); 3.8822 (0.5); 3.8674 (3.6);
3.8611 (2.9); 3.8552 (3.4); 3.8486 (4.0); 3.8432 (6.4); 3.8215 (6.0); 3.8162 (3.9); 3.8096 (3.2); 3.8037 (2.8);
3.7974 (3.4); 3.7827 (0.5); 3.7739 (1.1); 2.0465 (1.4); 1.6742 (2.5); 0.1081 (1.4); 0.0483 (0.5); 0.0375 (13.2);
0.0265 (0.4)
I-20: ¹H-NMR (300.2 MHz, CDCl₃):
δ = 8.3987 (3.8); 8.3947 (4.4); 8.3918 (4.3); 8.1252 (4.0); 8.0964 (4.2); 7.6197 (0.4); 7.5904 (8.2); 7.5862 (11.2);
7.5834 (11.0); 7.5580 (0.5); 7.3042 (19.2); 7.2761 (6.7); 7.2149 (4.6); 7.1862 (4.3); 6.5474 (3.3); 6.5220 (3.3);
5.3425 (0.5); 4.2591 (16.0); 3.8819 (1.1); 3.8733 (0.6); 3.8582 (3.6); 3.8516 (3.2); 3.8459 (3.7); 3.8344 (6.8);
3.8129 (6.6); 3.8004 (3.6); 3.7942 (3.1); 3.7887 (3.3); 3.7739 (0.6); 3.7650 (1.0); 1.6949 (0.7); 1.6545 (5.2);
1.2956 (1.6); 0.1108 (0.4); 0.0407 (19.0)
I-21: ¹H-NMR (300.2 MHz, CDCl₃):
δ = 7.7358 (5.6); 7.6303 (4.8); 7.6014 (5.3); 7.5586 (0.7); 7.5478 (7.2); 7.5406 (2.3); 7.5253 (2.4); 7.5180 (7.7);
7.5073 (0.8); 7.3051 (8.9); 7.0895 (4.8); 7.0813 (5.2); 7.0072 (6.0); 7.0047 (5.6); 6.9951 (1.0); 6.9842 (8.1);
6.9770 (2.4); 6.9616 (2.3); 6.9545 (7.0); 6.9437 (0.7); 6.9096 (3.0); 6.9013 (2.7); 6.8806 (2.8); 6.8723 (2.5);
5.3423 (0.7); 4.5295 (16.0); 3.8820 (1.4); 3.8604 (4.6); 3.8510 (2.9); 3.8439 (2.6); 3.8357 (3.5); 3.8056 (2.7);
3.7756 (3.6); 3.7674 (2.7); 3.7602 (2.9); 3.7508 (4.6); 3.7292 (1.5); 1.6962 (9.6); 1.2964 (1.4); 0.1120 (2.5);
0.0416 (7.8)
I-22: ¹H-NMR (300.2 MHz, CDCl₃):
δ = 7.6470 (7.1); 7.6026 (4.6); 7.5737 (4.9); 7.3973 (6.6); 7.3678 (7.7); 7.3022 (7.7); 7.0767 (4.9); 7.0687 (5.1);
7.0334 (7.8); 7.0040 (6.6); 6.8871 (2.9); 6.8790 (2.7); 6.8582 (2.7); 6.8501 (2.4); 6.6171 (0.4); 4.5915 (16.0);
3.8959 (1.4); 3.8738 (4.2); 3.8635 (3.8); 3.8564 (3.8); 3.8499 (4.4); 3.8380 (2.7); 3.8235 (2.7); 3.8118 (4.4);
3.8053 (3.8); 3.7982 (3.7); 3.7926 (3.8); 3.7877 (4.1); 3.7656 (1.2); 3.4690 (8.7); 1.8562 (0.4); 1.4637 (0.7);
1.2964 (4.7); 1.2601 (3.8); 1.1915 (0.4); 1.1572 (0.4); 0.9208 (0.6); 0.8965 (0.7); 0.8718 (0.5); 0.0414 (2.0)
I-23: ¹H-NMR (300.2 MHz, CDCl₃):
δ = 7.7564 (7.4); 7.7129 (4.5); 7.6788 (3.6); 7.6495 (9.7); 7.6204 (6.3); 7.4122 (1.2); 7.4039 (8.6); 7.4014 (10.0);
7.3941 (3.0); 7.3741 (10.6); 7.3717 (10.4); 7.3635 (1.2); 7.3605 (1.1); 7.3037 (30.0); 7.0772 (6.8); 7.0731 (5.7);
7.0691 (7.6); 7.0654 (4.6); 7.0563 (1.0); 7.0453 (6.8); 7.0378 (11.7); 7.0304 (3.3); 7.0160 (14.2); 7.0078 (11.1);
6.9276 (0.4); 6.9232 (0.3); 6.9105 (3.7); 6.9020 (5.2); 6.8933 (2.1); 6.8816 (3.4); 6.8731 (4.8); 6.8643 (1.9);
4.5757 (2.8); 4.5465 (1.9); 4.5268 (7.5); 4.4978 (4.7); 4.4769 (7.1); 4.4513 (4.6); 4.4280 (2.6); 4.4026 (1.6);
4.1756 (0.8); 4.1543 (1.5); 4.1343 (2.2); 4.1299 (1.6); 4.1140 (1.9); 4.1097 (2.1); 4.0896 (1.4); 4.0699 (0.4);
4.0086 (1.5); 3.9900 (1.9); 3.9820 (1.7); 3.9688 (3.3); 3.9637 (2.3); 3.9485 (3.0); 3.9438 (3.3); 3.9237 (2.4);
3.8464 (0.7); 3.8268 (1.0); 3.8194 (0.9); 3.8074 (0.8); 3.7997 (1.1); 3.7800 (0.7); 3.3429 (1.8); 3.3160 (3.2);
3.2889 (1.5); 3.0875 (3.0); 3.0624 (5.6); 3.0373 (2.7); 2.0879 (2.5); 1.6530 (9.4); 1.3712 (0.5); 1.3479 (1.1);
1.3252 (2.4); 1.3015 (7.8); 1.2777 (1.5); 1.2472 (0.6); 1.2095 (10.0); 1.1893 (9.8); 1.1610 (16.0); 1.1408 (15.8);
0.9450 (2.4); 0.9233 (7.1); 0.9002 (2.8); 0.0520 (0.9); 0.0413 (27.8); 0.0303 (1.0)
I-24: ¹H-NMR (499.9 MHz, CDCl₃):
δ = 7.6976 (1.0); 7.5828 (0.9); 7.5655 (0.9); 7.4857 (1.4); 7.4685 (1.5); 7.2600 (3.1); 7.0500 (0.9); 7.0454 (1.0);
6.9645 (2.3); 6.9470 (1.4); 6.8668 (0.5); 6.8621 (0.5); 6.8495 (0.5); 6.8448 (0.5); 4.4893 (3.1); 3.8288 (0.4);
3.8148 (1.4); 3.8011 (0.7); 3.7269 (0.6); 3.7133 (1.3); 3.6991 (0.4); 1.2565 (0.4); 0.2530 (16.0); 0.2382 (1.4); −0.0002 (3.1)
I-25: ¹H-NMR (300.2 MHz, CDCl₃):
δ = 7.6485 (5.8); 7.6466 (5.7); 7.6109 (5.0); 7.5820 (5.5); 7.5603 (0.9); 7.5516 (6.6); 7.5448 (2.2); 7.5293 (2.3);
7.5224 (7.3); 7.5137 (0.9); 7.2986 (16.3); 7.1051 (4.9); 7.0970 (5.3); 7.0326 (1.0); 7.0240 (7.8); 7.0170 (2.4);
7.0015 (2.3); 6.9946 (6.8); 6.9859 (0.8); 6.9120 (3.1); 6.9037 (2.9); 6.8831 (2.8); 6.8748 (2.7); 4.5915 (16.0);
3.8967 (1.3); 3.8750 (3.3); 3.8698 (2.8); 3.8642 (2.9); 3.8573 (2.9); 3.8504 (4.0); 3.8367 (2.2); 3.8236 (2.2);
3.8100 (4.1); 3.8030 (3.0); 3.7961 (2.9); 3.7906 (2.8); 3.7854 (3.4); 3.7635 (1.3); 3.4622 (9.9); 3.1049 (9.6);
2.0818 (0.8); 1.6760 (1.5); 1.4576 (0.4); 1.3696 (0.6); 1.3196 (1.3); 1.2909 (9.2); 1.2719 (1.5); 1.2569 (2.3);
1.1883 (0.6); 1.1514 (0.6); 1.1410 (0.4); 0.9159 (1.2); 0.8910 (1.2); 0.8667 (0.8); 0.1074 (0.4); 0.0464 (0.4);
0.0356 (11.3); 0.0247 (0.4)
I-26: ¹H-NMR (300.2 MHz, CDCl₃):
δ = 7.6618 (6.1); 7.6351 (3.8); 7.6115 (6.7); 7.6060 (8.2); 7.5827 (6.3); 7.3959 (1.1); 7.3851 (10.2); 7.3801 (3.4);
7.3777 (3.4); 7.3625 (4.1); 7.3552 (11.4); 7.3465 (1.3); 7.3442 (1.3); 7.2987 (13.2); 7.2867 (4.3); 7.0595 (6.1);
7.0552 (5.2); 7.0513 (7.2); 7.0471 (5.0); 7.0399 (1.3); 7.0288 (7.0); 7.0220 (11.4); 7.0147 (3.3); 7.0063 (2.3);
6.9992 (7.7); 6.9923 (8.5); 6.9813 (1.0); 6.8815 (3.6); 6.8731 (3.8); 6.8702 (3.1); 6.8617 (2.3); 6.8527 (3.4);
6.8443 (3.5); 6.8412 (2.8); 6.8327 (2.1); 6.6113 (0.4); 4.6348 (2.8); 4.5952 (1.9); 4.5865 (6.7); 4.5474 (4.6);
4.5245 (6.3); 4.5057 (4.5); 4.4763 (2.7); 4.4577 (1.5); 4.1881 (0.4); 4.1643 (1.1); 4.1519 (1.1); 4.1404 (1.3);
4.1317 (2.0); 4.1275 (1.6); 4.1115 (1.6); 4.1072 (2.1); 4.0870 (1.4); 4.0669 (0.4); 4.0330 (1.4); 4.0143 (2.0);
4.0066 (1.6); 3.9879 (2.1); 3.9718 (2.8); 3.9515 (2.6); 3.9466 (3.3); 3.9264 (2.4); 3.9178 (0.9); 3.9110 (0.4);
3.8980 (1.1); 3.8908 (1.0); 3.8785 (0.8); 3.8708 (1.2); 3.8515 (0.7); 3.4495 (16.0); 3.3538 (1.8); 3.3271 (3.2);
3.3003 (1.6); 3.1224 (2.9); 3.0974 (5.3); 3.0723 (2.6); 2.0763 (5.0); 1.4603 (0.4); 1.4538 (0.8); 1.3992 (0.4);
1.3662 (0.6); 1.3214 (1.0); 1.3142 (2.2); 1.2901 (7.5); 1.2866 (7.6); 1.2666 (2.2); 1.2491 (4.0); 1.2079 (10.3);
1.1877 (10.5); 1.1747 (15.5); 1.1545 (15.1); 1.1082 (0.5); 1.0880 (0.6); 1.0685 (0.4); 0.9107 (1.0); 0.8864 (1.0);
0.8634 (0.6); 0.1159 (2.4); 0.1038 (59.8); 0.0916 (2.4); 0.0789 (0.6); 0.0312 (6.4)
I-27: ¹H-NMR (300.2 MHz, CDCl₃):
δ = 7.7551 (6.0); 7.6320 (3.2); 7.6029 (3.5); 7.4146 (4.8); 7.4095 (9.4); 7.3873 (2.6); 7.3798 (8.6); 7.3689 (1.0);
7.2989 (12.1); 7.1042 (2.3); 7.0957 (2.2); 7.0752 (2.1); 7.0666 (2.0); 7.0443 (1.0); 7.0333 (8.4); 7.0260 (2.7);
7.0109 (2.4); 7.0036 (7.4); 6.9925 (0.9); 6.9802 (6.0); 6.9775 (6.3); 4.3670 (16.0); 3.8583 (0.6); 3.8314 (2.0);
3.8252 (2.8); 3.8197 (3.9); 3.8121 (9.7); 3.8008 (9.7); 3.7936 (4.1); 3.7879 (2.9); 3.7801 (2.1); 3.7550 (0.6);
2.0828 (0.8); 1.6614 (5.8); 1.3203 (0.3); 1.2904 (1.3); 1.2727 (0.4); 0.0470 (0.4); 0.0362 (11.6); 0.0254 (0.6)
I-28: ¹H-NMR (300.2 MHz, CDCl₃):
δ = 7.6821 (6.6); 7.5867 (3.4); 7.5576 (3.7); 7.4071 (5.4); 7.4018 (10.1); 7.3796 (2.7); 7.3726 (8.1); 7.3616 (0.9);
7.2989 (7.2); 7.2670 (6.6); 7.0780 (2.4); 7.0696 (2.3); 7.0489 (2.2); 7.0401 (2.4); 7.0258 (8.2); 7.0188 (2.6);
7.0032 (2.5); 6.9963 (6.9); 6.9852 (0.7); 4.4355 (16.0); 4.1926 (0.5); 4.1687 (1.4); 4.1449 (1.4); 4.1212 (0.6);
3.8877 (1.1); 3.8766 (0.7); 3.8646 (3.6); 3.8588 (3.2); 3.8534 (3.4); 3.8468 (3.8); 3.8407 (5.1); 3.8137 (5.1);
3.8078 (3.7); 3.8011 (3.4); 3.7957 (3.2); 3.7900 (3.6); 3.7780 (0.7); 3.7669 (1.0); 3.3968 (9.4); 2.0810 (6.7);
1.7104 (3.8); 1.4573 (0.3); 1.3185 (2.0); 1.2944 (5.6); 1.2709 (2.1); 1.2552 (1.7); 1.1869 (0.4); 1.1503 (0.3);
0.9704 (0.4); 0.9151 (0.5); 0.8896 (0.5); 0.8644 (0.3); 0.1981 (0.6); 0.1854 (0.4); 0.1807 (0.4); 0.1765 (0.4);
0.1542 (0.7); 0.1469 (1.1); 0.1381 (0.5); 0.1066 (0.6); 0.0346 (6.1)
I-29: ¹H-NMR (300.2 MHz, CDCl₃):
δ = 7.7403 (5.4); 7.6821 (4.4); 7.6649 (5.6); 7.6537 (5.2); 7.6362 (5.4); 7.2982 (7.2); 7.1552 (6.8); 7.1477 (8.4);
7.1220 (4.4); 7.0038 (5.6); 7.0014 (5.0); 6.9630 (3.0); 6.9548 (2.8); 6.9342 (2.8); 6.9259 (2.6); 4.5372 (16.0);
4.1916 (0.4); 4.1678 (1.4); 4.1440 (1.4); 4.1204 (0.6); 3.8928 (1.4); 3.8713 (4.5); 3.8616 (2.8); 3.8545 (2.7);
3.8464 (3.6); 3.8171 (2.7); 3.7877 (3.7); 3.7795 (2.8); 3.7724 (3.0); 3.7628 (4.6); 3.7412 (1.5); 2.0799 (6.9);
2.0424 (0.4); 1.7316 (0.6); 1.3175 (1.8); 1.2936 (4.5); 1.2699 (1.9); 0.9695 (0.4); 0.0337 (5.3)
I-30: ¹H-NMR (300.2 MHz, CDCl₃):
δ = 7.6778 (4.3); 7.6500 (10.1); 7.6356 (5.4); 7.6068 (5.5); 7.2985 (13.0); 7.1467 (9.2); 7.1388 (7.0); 7.1167 (4.2);
6.9433 (3.2); 6.9351 (2.9); 6.9145 (2.9); 6.9063 (2.7); 4.5994 (16.0); 4.1920 (0.4); 4.1681 (1.2); 4.1443 (1.2);
4.1206 (0.4); 3.9096 (1.2); 3.8939 (1.1); 3.8877 (3.2); 3.8820 (2.7); 3.8764 (2.9); 3.8696 (3.0); 3.8632 (4.2);
3.8534 (2.6); 3.8381 (2.5); 3.8284 (4.3); 3.8219 (3.1); 3.8151 (3.0); 3.8095 (2.8); 3.8040 (3.4); 3.7977 (1.2);
3.7818 (1.3); 3.4631 (9.7); 2.0803 (5.6); 1.7139 (1.1); 1.3178 (1.6); 1.2940 (4.0); 1.2903 (2.2); 1.2702 (1.7);
1.2548 (1.7); 1.1862 (0.3); 1.1501 (0.3); 0.0341 (7.0)
I-31: ¹H-NMR (300.2 MHz, CDCl₃):
δ = 7.7599 (6.0); 7.7569 (5.9); 7.7444 (0.5); 7.7135 (6.8); 7.6860 (10.8); 7.6578 (10.5); 7.3953 (0.6); 7.2995
(13.4); 7.1498 (11.5); 7.1459 (7.2); 7.1418 (8.0); 7.1375 (5.0); 7.1223 (5.2); 7.0971 (0.3); 7.0130 (6.4); 7.0098
(6.3); 7.0015 (4.0); 6.9982 (3.8); 6.9686 (3.5); 6.9601 (4.1); 6.9503 (2.0); 6.9398 (3.2); 6.9314 (3.8); 6.9213 (2.4);
6.9153 (0.9); 5.3740 (0.3); 5.3709 (0.4); 5.3002 (0.3); 4.5828 (2.4); 4.5628 (0.5); 4.5581 (0.9); 4.5522 (1.8);
4.5338 (7.4); 4.5033 (4.4); 4.4884 (6.6); 4.4628 (4.3); 4.4395 (2.3); 4.4141 (1.4); 4.1794 (0.4); 4.1689 (0.8);
4.1647 (1.2); 4.1445 (2.4); 4.1399 (1.6); 4.1342 (1.0); 4.1245 (1.8); 4.1197 (2.1); 4.0996 (1.4); 4.0799 (0.6);
4.0225 (1.4); 4.0038 (2.2); 3.9959 (1.7); 3.9807 (3.3); 3.9674 (0.9); 3.9609 (2.6); 3.9559 (3.3); 3.9435 (0.5);
3.9358 (2.4); 3.8673 (0.7); 3.8476 (0.9); 3.8399 (0.8); 3.8281 (0.7); 3.8202 (1.0); 3.8008 (0.7); 3.6892 (0.3);
3.6688 (0.3); 3.3546 (1.6); 3.3277 (2.9); 3.3006 (1.4); 3.1010 (2.8); 3.0758 (5.3); 3.0506 (2.6); 2.4584 (0.3);
2.4331 (1.0); 2.4079 (1.1); 2.4019 (0.7); 2.3827 (0.5); 2.3767 (0.8); 2.2220 (0.5); 2.2085 (0.5); 2.1409 (2.4);
2.1296 (0.7); 2.1170 (2.1); 2.0962 (0.4); 2.0798 (1.2); 1.7275 (4.9); 1.3667 (0.4); 1.3607 (0.3); 1.3427 (0.7);
1.3180 (1.4); 1.3018 (3.6); 1.2944 (3.7); 1.2894 (3.3); 1.2860 (2.9); 1.2673 (1.8); 1.2609 (3.8); 1.2400 (3.4);
1.2297 (1.0); 1.2238 (2.0); 1.2161 (9.4); 1.1959 (9.7); 1.1868 (3.1); 1.1711 (16.0); 1.1617 (2.8); 1.1509 (15.6);
1.1365 (1.1); 0.9571 (0.3); 0.9382 (1.5); 0.9167 (4.3); 0.8934 (1.7); 0.0458 (0.4); 0.0349 (12.1); 0.0241 (0.5)
I-32: ¹H-NMR (300.2 MHz, CDCl₃):
δ = 7.7529 (6.0); 7.7260 (0.4); 7.7072 (7.8); 7.6899 (6.3); 7.6783 (10.1); 7.6733 (8.6); 7.6707 (9.0); 7.6653 (8.2);
7.6612 (7.4); 7.6554 (6.0); 7.2985 (10.7); 7.1635 (11.0); 7.1557 (7.9); 7.1342 (4.8); 6.9825 (3.0); 6.9743 (2.8);
6.9619 (1.8); 6.9537 (4.2); 6.9455 (2.7); 6.9330 (1.5); 6.9248 (1.4); 5.3349 (16.0); 4.6916 (2.1); 4.6538 (1.0);
4.6428 (6.1); 4.6053 (4.3); 4.5987 (6.1); 4.5824 (3.7); 4.5498 (2.0); 4.5338 (0.8); 4.1863 (0.9); 4.1660 (1.7);
4.1618 (1.3); 4.1456 (1.4); 4.1415 (1.7); 4.1212 (1.1); 4.0654 (1.1); 4.0467 (1.4); 4.0382 (1.3); 4.0196 (1.4);
4.0064 (2.4); 3.9859 (2.2); 3.9810 (2.7); 3.9606 (2.0); 3.8573 (0.5); 3.8379 (0.7); 3.8296 (0.6); 3.8182 (0.6);
3.8100 (0.8); 3.7903 (0.5); 3.3832 (1.3); 3.3556 (2.3); 3.3280 (1.1); 3.0705 (2.5); 3.0453 (4.5); 3.0200 (2.2);
1.6780 (9.2); 1.2878 (0.9); 1.2777 (0.4); 1.2383 (7.2); 1.2180 (7.2); 1.1532 (13.1); 1.1331 (12.9); 1.1177 (0.6);
0.0436 (0.3); 0.0327 (9.6); 0.0219 (0.4)
I-33: ¹H-NMR (300.2 MHz, CDCl₃):
δ = 7.6982 (6.2); 7.6553 (6.8); 7.6529 (5.5); 7.6265 (5.1); 7.5975 (5.4); 7.4128 (0.7); 7.4018 (7.3); 7.3944 (2.3);
7.3883 (0.4); 7.3794 (2.5); 7.3719 (8.1); 7.3609 (0.9); 7.2985 (6.5); 7.0917 (4.8); 7.0835 (5.2); 7.0503 (0.9);
7.0393 (8.6); 7.0318 (2.5); 7.0169 (2.3); 7.0095 (6.8); 6.9985 (0.6); 6.9045 (3.2); 6.8962 (2.9); 6.8755 (2.9);
6.8672 (2.7); 5.3346 (2.5); 4.6318 (16.0); 3.9008 (1.7); 3.8773 (4.9); 3.8704 (2.9); 3.8632 (2.6); 3.8540 (3.4);
3.8225 (1.0); 3.8025 (1.0); 3.7710 (3.5); 3.7617 (2.6); 3.7545 (3.0); 3.7481 (5.1); 3.7240 (1.7); 3.0485 (2.2);
2.9757 (1.7); 2.1179 (1.7); 2.0796 (1.1); 1.6818 (3.6); 1.3170 (0.5); 1.2930 (1.4); 1.2882 (1.4); 1.2694 (0.4);
0.9146 (0.4); 0.0331 (6.5)
I-34: ¹H-NMR (300.2 MHz, CDCl₃):
δ = 7.7309 (0.6); 7.7132 (6.3); 7.6923 (4.6); 7.6764 (6.0); 7.6639 (11.6); 7.6476 (5.8); 7.2988 (11.6); 7.1742 (5.9);
7.1661 (9.9); 7.1371 (4.2); 6.9751 (3.2); 6.9669 (3.0); 6.9462 (3.0); 6.9380 (2.7); 5.3366 (9.0); 4.6492 (16.0);
3.9201 (1.8); 3.8962 (4.9); 3.8897 (3.0); 3.8825 (2.6); 3.8733 (3.5); 3.8417 (1.0); 3.8202 (1.0); 3.7888 (3.6);
3.7795 (2.7); 3.7723 (3.0); 3.7659 (5.0); 3.7417 (1.7); 2.0820 (1.0); 1.6449 (2.3); 1.3194 (0.4); 1.2954 (1.1);
1.2900 (1.1); 1.2717 (0.4); 0.0461 (0.4); 0.0353 (12.1); 0.0244 (0.5)
I-35: ¹H-NMR (300.2 MHz, CDCl₃):
δ = 7.7552 (3.6); 7.7285 (8.6); 7.6582 (6.9); 7.6314 (0.4); 7.6022 (0.4); 7.4343 (4.4); 7.4257 (5.0); 7.4202 (8.0);
7.4129 (3.0); 7.4085 (1.6); 7.3978 (2.7); 7.3905 (8.7); 7.3791 (1.7); 7.2988 (11.6); 7.1455 (2.3); 7.1369 (2.2);
7.1165 (2.1); 7.1078 (2.1); 7.0613 (0.9); 7.0502 (8.8); 7.0429 (2.7); 7.0319 (1.5); 7.0278 (2.5); 7.0206 (7.3);
7.0096 (0.9); 7.0027 (0.9); 6.9790 (0.7); 5.3364 (0.4); 4.4515 (16.0); 4.3662 (1.8); 4.1926 (0.8); 4.1688 (2.5);
4.1450 (2.5); 4.1214 (1.2); 4.0998 (0.8); 4.0775 (0.4); 3.8373 (0.6); 3.8239 (0.6); 3.8112 (3.0); 3.8041 (3.0);
3.7987 (4.8); 3.7911 (10.0); 3.7796 (9.9); 3.7720 (3.9); 3.7660 (2.7); 3.7599 (2.0); 3.7330 (0.6); 2.1253 (0.4);
2.0949 (0.4); 2.0815 (14.0); 1.6501 (2.7); 1.6208 (0.6); 1.4035 (0.3); 1.3378 (0.3); 1.3187 (3.2); 1.2949 (6.8);
1.2711 (3.1); 0.9948 (0.7); 0.9785 (0.4); 0.9704 (1.3); 0.9561 (0.4); 0.9460 (0.6); 0.0454 (0.4); 0.0345 (11.8);
0.0238 (0.4)
I-RP-01: ¹H-NMR (300.2 MHz, CDCl₃):
δ = 7.6072 (1.1); 7.5478 (0.8); 7.5188 (0.9); 7.3771 (1.2); 7.3699 (0.4); 7.3547 (0.4); 7.3474 (1.4); 7.2384
(1.1); 7.0518 (0.9); 7.0436 (0.9); 7.0166 (1.5); 7.0094 (0.4); 6.9941 (0.4); 6.9869 (1.2); 6.8593 (0.5); 6.8510
(0.5); 6.8303 (0.5); 6.8221 (0.4); 4.5629 (2.5); 3.8615 (0.4); 3.8541 (0.5); 3.8407 (1.6); 3.8281 (1.6); 3.8143
(0.5); 3.8076 (0.4); 0.2796 (0.6); 0.2681 (16.0); 0.2563 (0.8)
I-RP-02: ¹H-NMR (300.2 MHz, CDCl₃):
δ = 7.6189 (0.9); 7.5614 (0.8); 7.5325 (0.9); 7.5268 (1.2); 7.5201 (0.4); 7.5043 (0.4); 7.4976 (1.2); 7.3049
(0.7); 7.2535 (0.9); 7.0717 (0.8); 7.0635 (0.8); 7.0041 (1.2); 6.9974 (0.4); 6.9816 (0.4); 6.9750 (1.1); 6.8812
(0.5); 6.8729 (0.4); 6.8523 (0.4); 6.8440 (0.4); 4.5734 (2.2); 3.8754 (0.4); 3.8685 (0.4); 3.8630 (0.5); 3.8531
(1.2); 3.8378 (1.2); 3.8278 (0.5); 3.8222 (0.4); 3.8155 (0.4); 2.0834 (0.7); 1.2973 (0.4); 0.3028 (0.5); 0.2915
(16.0); 0.2767 (15.5); 0.2649 (0.7); 0.0380 (0.6)
I-RP-03: ¹H-NMR (300.2 MHz, CDCl₃):
δ = 7.6320 (0.4); 7.5950 (0.5); 7.5669 (1.2); 7.5386 (0.7); 7.3904 (0.9); 7.3877 (1.2); 7.3804 (0.4); 7.3651
(0.5); 7.3605 (1.1); 7.3579 (1.3); 7.2988 (2.0); 7.2525 (0.5); 7.0511 (0.8); 7.0485 (0.7); 7.0430 (1.0); 7.0405
(0.7); 7.0322 (0.9); 7.0246 (1.4); 7.0172 (0.4); 7.0023 (1.0); 6.9948 (1.0); 6.8698 (0.4); 6.8614 (0.4); 6.8587
(0.4); 6.8409 (0.4); 6.8325 (0.4); 6.8297 (0.3); 4.5677 (0.7); 4.5265 (0.6); 4.5163 (0.7); 4.5012 (0.6); 3.9768
(0.4); 3.9564 (0.4); 3.9518 (0.4); 3.3319 (0.4); 3.1180 (0.6); 1.7133 (0.5); 1.2145 (2.1); 1.1943 (2.0); 0.2864
(0.9); 0.2832 (0.8); 0.2743 (12.2); 0.2716 (16.0); 0.2597 (0.7); 0.0350 (1.8)
I-RP-04: ¹H-NMR (300.2 MHz, CDCl₃):
δ = 7.6738 (0.8); 7.5379 (0.4); 7.5088 (0.5); 7.4021 (1.7); 7.3945 (1.0); 7.3796 (0.4); 7.3722 (1.3); 7.2985
(1.7); 7.2317 (0.8); 7.0626 (0.3); 7.0540 (0.3); 7.0331 (0.4); 7.0274 (1.5); 7.0201 (0.4); 7.0050 (0.4); 6.9976
(1.1); 4.4227 (2.1); 3.8832 (0.5); 3.8783 (0.4); 3.8727 (0.4); 3.8660 (0.4); 3.8583 (0.6); 3.8136 (0.6); 3.8060
(0.4); 3.7993 (0.4); 3.7937 (0.4); 3.7890 (0.5); 2.0441 (0.5); 1.6867 (1.2); 0.2699 (0.6); 0.2670 (0.4); 0.2581
(16.0); 0.2489 (0.4); 0.2463 (0.6); 0.0352 (1.4)
I-RP-05: ¹H-NMR (300.2 MHz, CDCl₃):
δ = 7.6760 (0.7); 7.6476 (0.8); 7.6235 (0.9); 7.6215 (0.9); 7.5911 (0.8); 7.5623 (0.9); 7.2987 (1.3); 7.2522
(0.9); 7.2498 (0.9); 7.1414 (0.8); 7.1339 (1.0); 7.1257 (0.9); 7.1134 (0.7); 6.9261 (0.5); 6.9179 (0.5); 6.8972
(0.4); 6.8890 (0.4); 4.5822 (2.3); 3.8841 (0.4); 3.8784 (0.6); 3.8708 (1.4); 3.8599 (1.4); 3.8521 (0.6); 3.8464
(0.4); 2.0809 (0.5); 1.2946 (0.3); 0.2848 (0.6); 0.2731 (16.0); 0.2614 (0.6); 0.0347 (1.2)

### BIOLOGICAL EXAMPLES

### Example A

### In Vivo Preventive Test on Botrytis cinerea (Grey Mould)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of gherkin were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Botrytis cinerea spores. The contaminated gherkin plants were incubated for 4 to 5 days at 17° C. and at 90% relative humidity.

The test was evaluated 4 to 5 days after the inoculation. 0% means an efficacy which corresponds to that of the control plants while an efficacy of 100% means that no disease was observed.

In this test the following compounds according to the invention showed efficacy between 70% and 79% at a concentration of 500 ppm of active ingredient: I-03.

In this test the following compounds according to the invention showed efficacy between 80% and 89% at a concentration of 500 ppm of active ingredient: I-08; I-29.

In this test the following compounds according to the invention showed efficacy between 90% and 100% at a concentration of 500 ppm of active ingredient: I-02; I-07; I-11; I-13; I-14; I-18; I-20; I-21; I-22; I-28; I-30

### Example B

### In Vivo Preventive Test on Puccinia recondita (Brown Rust on Wheat)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of wheat were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Puccinia recondita spores. The contaminated wheat plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test the following compounds according to the invention showed efficacy between 70% and 79% at a concentration of 500 ppm of active ingredient: I-01; I-04; I-05; I-19.

In this test the following compounds according to the invention showed efficacy between 80% and 89% at a concentration of 500 ppm of active ingredient: I-03; I-06; I-08; I-13; I-14; I-15; I-23; I-28.

In this test the following compounds according to the invention showed efficacy between 90% and 100% at a concentration of 500 ppm of active ingredient: I-02; I-07; I-09; I-10; I-11; I-12; I-16; I-18; I-20; I-21; I-22; I-25; I-26; I-29; I-30.

### Example C

### In Vivo Preventive Test on Pyrenophora teres (Net Blotch on Barley)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of barley were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Pyrenophora teres spores. The contaminated barley plants were incubated for 48 hours at 20° C. and at 100% relative humidity and then for 12 days at 20° C. and at 70-80% relative humidity.

The test was evaluated 14 days after the inoculation. 0% means an efficacy which corresponds to that of the control plants while an efficacy of 100% means that no disease was observed.

In this test the following compounds according to the invention showed efficacy between 70% and 79% at a concentration of 500 ppm of active ingredient: I-05; I-06; I-09; I-25.

In this test the following compounds according to the invention showed efficacy between 80% and 89% at a concentration of 500 ppm of active ingredient: I-08; I-11; I-13; I-14; I-21; I-23; I-28; I-29; I-30.

In this test the following compounds according to the invention showed efficacy between 90% and 100% at a concentration of 500 ppm of active ingredient: I-02; I-12; I-22; I-26.

### Example D

### In Vivo Preventive Test on Septoria tritici (Leaf Spot on Wheat)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of wheat were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Septoria tritici spores. The contaminated wheat plants were incubated for 72 hours at 18° C. and at 100% relative humidity and then for 21 days at 20° C. and at 90% relative humidity.

The test was evaluated 24 days after the inoculation. 0% means an efficacy which corresponds to that of the control plants while an efficacy of 100% means that no disease was observed.

In this test the following compounds according to the invention showed efficacy between 70% and 79% at a concentration of 500 ppm of active ingredient: I-22; I-24.

In this test the following compounds according to the invention showed efficacy between 90% and 100% at a concentration of 500 ppm of active ingredient: I-01; I-02; I-03; I-04; I-05; I-06; I-07; I-08; I-09; I-10; I-11; I-13; I-14; I-15; I-16; I-17; I-18; I-19; I-20; I-21; I-23; I-25; I-26; I-28; I-29; I-30.

### Example E

### In Vivo Preventive Test on Sphaerotheca fuliginea (Powdery Mildew on Cucurbits)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of gherkin were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Sphaerotheca fuliginea spores. The contaminated gherkin plants were incubated for 72 hours at 18° C. and at 100% relative humidity and then for 12 days at 20° C. and at 70-80% relative humidity.

The test was evaluated 15 days after the inoculation. 0% means an efficacy which corresponds to that of the control plants while an efficacy of 100% means that no disease was observed.

In this test the following compounds according to the invention showed efficacy between 70% and 79% at a concentration of 500 ppm of active ingredient: I-22.

In this test the following compounds according to the invention showed efficacy between 90% and 100% at a concentration of 500 ppm of active ingredient: I-01; I-02; I-03; I-04; I-05; I-06; I-07; I-08; I-09; I-10; I-11; I-12; I-13; I-14; I-15; I-16; I-17; I-18; I-19; I-20; I-21; I-23; I-24; I-26; I-28; I-29; I-30.

### Example F

### In Vivo Preventive Test on Uromyces appendiculatus (Bean Rust)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of bean were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Uromyces appendiculatus spores. The contaminated bean plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test the following compounds according to the invention showed efficacy between 90% and 100% at a concentration of 500 ppm of active ingredient: I-01; I-02; I-03; I-04; I-05; I-06; I-07; I-08; I-09; I-10; I-11; I-12; I-13; I-14; I-15; I-16; I-17; I-18; I-19; I-20; I-21; I-22; I-23; I-25; I-26; I-28; I-29; I-30.

### Example G

### In Vivo Preventive Test on Alternaria Test (Tomatoes)

| | | |
|---|---|---|
| Solvent: | 24.5 | parts by weight of acetone |
| | 24.5 | parts by weight of dimethylacetamide |
| Emulsifier: | 1 | part by weight of alkylaryl polyglycol ether |

To produce a suitable preparation of active compound, 1 part by weight of active compound was mixed with the stated amounts of solvent and emulsifier, and the concentrate was diluted with water to the desired concentration.

To test for preventive activity, young plants were sprayed with the preparation of active compound at the stated rate of application. After the spray coating had dried on, the plants were inoculated with an aqueous spore suspension of Alternaria solani. The plants were then placed in an incubation cabinet at approximately 20° C. and a relative atmospheric humidity of 100%.

The test was evaluated 3 days after the inoculation. 0% means an efficacy which corresponds to that of the untreated control while an efficacy of 100% means that no disease is observed.

In this test the following compounds according to the invention showed efficacy between 80% and 89% at a concentration of 100 ppm of active ingredient: I-06; I-16; I-18; I-20; I-25; I-28.

In this test the following compounds according to the invention showed efficacy between 90% and 100% at a concentration of 100 ppm of active ingredient: I-01; I-02; I-03; I-04; I-07; I-11; I-13; I-22; I-26; I-30.

### Example H

### In Vivo Preventive Test on Botrytis Test (Beans)

| | | |
|---|---|---|
| Solvent: | 24.5 | parts by weight of acetone |
| | 24.5 | parts by weight of dimethylacetamide |
| Emulsifier: | 1 | part by weight of alkylaryl polyglycol ether |

To produce a suitable preparation of active compound, 1 part by weight of active compound was mixed with the stated amounts of solvent and emulsifier, and the concentrate was diluted with water to the desired concentration.

To test for preventive activity, young plants were sprayed with the preparation of active compound. After the spray coating had dried on, 2 small pieces of agar covered with growth of Botrytis cinerea were placed on each leaf. The inoculated plants were placed in a darkened chamber at 20° C. and a relative atmospheric humidity of 100%.

2 days after the inoculation, the size of the lesions on the leaves was evaluated. 0% means an efficacy which corresponds to that of the untreated control, while an efficacy of 100% means that no disease is observed.

In this test the following compounds according to the invention showed efficacy between 80% and 89% at a concentration of 100 ppm of active ingredient: I-20.

In this test the following compounds according to the invention showed efficacy between 90% and 100% at a concentration of 100 ppm of active ingredient: I-01; I-02; I-03; I-04; I-06; I-07; I-11; I-13; I-16; I-18; I-22; I-25; I-28.

### Example I

### In Vivo Preventive Test on Phakopsora Test (Soybeans)

| | | |
|---|---|---|
| Solvent: | 24.5 | parts by weight of acetone |
| | 24.5 | parts by weight of dimethylacetamide |
| Emulsifier: | 1 | part by weight of alkylaryl polyglycol ether |

To produce a suitable preparation of active compound, 1 part by weight of active compound was mixed with the stated amounts of solvent and emulsifier, and the concentrate was diluted with water to the desired concentration.

To test for preventive activity, young plants were sprayed with the preparation of active compound at the stated rate of application. After the spray coating had dried on, the plants were inoculated with an aqueous spore suspension of the causal agent of soybean rust (Phakopsora pachyrhizi) and stay for 24 h without light in an incubation cabinet at approximately 24° C. and a relative atmospheric humidity of 95%.

The plants remained in the incubation cabinet at approximately 24° C. and a relative atmospheric humidity of approximately 80% and a day/night interval of 12 h.

The test was evaluated 7 days after the inoculation. 0% means an efficacy which corresponds to that of the untreated control, while an efficacy of 100% means that no disease is observed.

In this test the following compounds according to the invention showed efficacy between 70% and 79% at a concentration of 100 ppm of active ingredient: I-03.

In this test the following compounds according to the invention showed efficacy between 80% and 89% at a concentration of 100 ppm of active ingredient: I-01; I-20; I-25.

In this test the following compounds according to the invention showed efficacy between 90% and 100% at a concentration of 100 ppm of active ingredient: I-02; I-04; I-06; I-07; I-11; I-16; I-18; I-22; I-28.

### Example J

### In Vivo Preventive Test on Venturia Test (Apples)

| | | |
|---|---|---|
| Solvent: | 24.5 | parts by weight of acetone |
| | 24.5 | parts by weight of dimethylacetamide |
| Emulsifier: | 1 | part by weight of alkylaryl polyglycol ether |

To produce a suitable preparation of active compound, 1 part by weight of active compound was mixed with the stated amounts of solvent and emulsifier, and the concentrate was diluted with water to the desired concentration.

To test for preventive activity, young plants were sprayed with the preparation of active compound at the stated rate of application. After the spray coating had dried on, the plants were inoculated with an aqueous conidia suspension of the causal agent of apple scab (Venturia inaequalis) and then remained for 1 day in an incubation cabinet at approximately 20° C. and a relative atmospheric humidity of 100%.

The plants were then placed in a greenhouse at approximately 21° C. and a relative atmospheric humidity of approximately 90%.

The test was evaluated 10 days after the inoculation. 0% means an efficacy which corresponds to that of the untreated control, while an efficacy of 100% means that no disease is observed.

In this test the following compounds according to the invention showed efficacy between 90% and 100% at a concentration of 100 ppm of active ingredient: I-01; I-02; I-03; I-04; I-06; I-07; I-11; I-13; I-16; I-18; I-20; I-22; I-25; I-26; I-28; I-30.

### Example K

### In Vivo Preventive Blumeria Test (Barley)

| | | |
|---|---|---|
| Solvent: | 49 | parts by weight of N,N-dimethylacetamide |
| Emulsifier: | 1 | part by weight of alkylaryl polyglycol ether |

To produce a suitable preparation of active compound, 1 part by weight of active compound or active compound combination was mixed with the stated amounts of solvent and emulsifier, and the concentrate was diluted with water to the desired concentration.

To test for preventive activity, young plants were sprayed with the preparation of active compound or active compound combination at the stated rate of application.

After the spray coating had been dried, the plants were dusted with spores of Blumeria graminis f.sp. hordei.

The plants were placed in the greenhouse at a temperature of approximately 18° C. and a relative atmospheric humidity of approximately 80% to promote the development of mildew pustules.

The test was evaluated 7 days after the inoculation. 0% means an efficacy which corresponds to that of the untreated control, while an efficacy of 100% means that no disease is observed.

In this test the following compounds according to the invention showed efficacy between 70% and 79% at a concentration of 500 ppm of active ingredient: I-30.

In this test the following compounds according to the invention showed efficacy between 80% and 89% at a concentration of 500 ppm of active ingredient: I-01; I-13; I-19.

In this test the following compounds according to the invention showed efficacy between 90% and 100% at a concentration of 500 ppm of active ingredient: I-14; I-16; I-18; I-20; I-25.

### Example L

### In Vivo Preventive Leptosphaeria nodorum Test (Wheat)

| | | |
|---|---|---|
| Solvent: | 49 | parts by weight of N,N-dimethylacetamide |
| Emulsifier: | 1 | part by weight of alkylaryl polyglycol ether |

To produce a suitable preparation of active compound, 1 part by weight of active compound or active compound combination was mixed with the stated amounts of solvent and emulsifier, and the concentrate was diluted with water to the desired concentration.

To test for preventive activity, young plants were sprayed with the preparation of active compound or active compound combination at the stated rate of application.

After the spray coating had been dried, the plants were sprayed with a spore suspension of Leptosphaeria nodorum. The plants remained for 48 hours in an incubation cabinet at approximately 20° C. and a relative atmospheric humidity of approximately 100%.

The plants were placed in the greenhouse at a temperature of approximately 25° C. and a relative atmospheric humidity of approximately 80%.

The test was evaluated 8 days after the inoculation. 0% means an efficacy which corresponds to that of the untreated control, while an efficacy of 100% means that no disease is observed.

In this test the following compounds according to the invention showed efficacy between 70% and 79% at a concentration of 500 ppm of active ingredient: I-07.

In this test the following compounds according to the invention showed efficacy between 80% and 89% at a concentration of 500 ppm of active ingredient: I-01; I-03; I-04; I-08; I-11.

In this test the following compounds according to the invention showed efficacy between 90% and 100% at a concentration of 500 ppm of active ingredient: I-12.

### Example RP-A

### In Vivo Preventive Test on Puccinia recondita (Brown Rust on Wheat)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of wheat were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Puccinia recondita spores. The contaminated wheat plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test the following compounds according to the invention showed efficacy between 80% and 89% at a concentration of 500 ppm of active ingredient: I-RP-01; I-RP-04.

### Example RP-B

### In Vivo Preventive Test on Septoria tritici (Leaf Spot on Wheat)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of wheat were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Septoria tritici spores. The contaminated wheat plants were incubated for 72 hours at 18° C. and at 100% relative humidity and then for 21 days at 20° C. and at 90% relative humidity.

The test was evaluated 24 days after the inoculation. 0% means an efficacy which corresponds to that of the control plants while an efficacy of 100% means that no disease was observed.

In this test the following compounds according to the invention showed efficacy between 80% and 89% at a concentration of 500 ppm of active ingredient: I-RP-04.

In this test the following compounds according to the invention showed efficacy between 90% and 100% at a concentration of 500 ppm of active ingredient: I-RP-01.

### Example RP-C

### In Vivo Preventive Test on Sphaerotheca fuliginea (Powdery Mildew on Cucurbits)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of gherkin were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Sphaerotheca fuliginea spores. The contaminated gherkin plants were incubated for 72 hours at 18° C. and at 100% relative humidity and then for 12 days at 20° C. and at 70-80% relative humidity.

The test was evaluated 15 days after the inoculation. 0% means an efficacy which corresponds to that of the control plants while an efficacy of 100% means that no disease was observed.

In this test the following compounds according to the invention showed efficacy between 90% and 100% at a concentration of 500 ppm of active ingredient: I-RP-01; I-RP-03; I-RP-04.

### Example RP-D

### In Vivo Preventive Test on Uromyces appendiculatus (Bean Rust)

| | | |
|---|---|---|
| Solvent: | 5% | by volume of Dimethyl sulfoxide |
| | 10% | by volume of Acetone |
| Emulsifier: | 1 μl | of Tween ® 80 per mg of active ingredient |

The active ingredients were made soluble and homogenized in a mixture of Dimethyl sulfoxide/Acetone//Tween® 80 and then diluted in water to the desired concentration.

The young plants of bean were treated by spraying the active ingredient prepared as described above. Control plants were treated only with an aqueous solution of Acetone/Dimethyl sulfoxide/Tween® 80.

After 24 hours, the plants were contaminated by spraying the leaves with an aqueous suspension of Uromyces appendiculatus spores. The contaminated bean plants were incubated for 24 hours at 20° C. and at 100% relative humidity and then for 10 days at 20° C. and at 70-80% relative humidity.

The test was evaluated 11 days after the inoculation. 0% means an efficacy which corresponds to that of the control plants while an efficacy of 100% means that no disease was observed.

In this test the following compounds according to the invention showed efficacy between 90% and 100% at a concentration of 500 ppm of active ingredient: I-RP-01.

1. Imidazole derivative of formula (I) 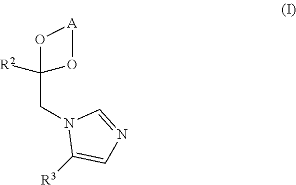
wherein
A represents a linear C₁-C₆-alkylene bridge which may be substituted by 1, 2 or up to the maximum possible number of identical or different groups R¹, wherein
R¹ represents halogen, C₁-C₆-alkyl, C₂-C₆-alkenyl, C₂-C₆-alkynyl, C₃-C₈-cycloalkyl, C₃-C₈-cycloalkyl-C₁-C₄-alkyl, C₁-C₆-alkoxy, C₁-C₆-alkylthio, phenyl, phenyl-C₁-C₄-alkyl, phenyl-C₂-C₄-alkenyl or phenyl-C₂-C₄-alkynyl;
wherein the aliphatic moieties, excluding cycloalkyl moieties, of R¹ may carry 1, 2, 3 or up to the maximum possible number of identical or different groups R^{a} which independently of one another are selected from
R^{a} halogen, CN, nitro, phenyl, C₁-C₄-alkoxy and C₁-C₄-haloalkoxy; wherein the phenyl may be substituted by 1, 2, 3, 4 or 5 substituents selected independently of one another from halogen, CN, nitro, C₁-C₄-alkyl, C₁-C₄-alkoxy, C₁-C₄-haloalkyl, C₁-C₄-haloalkoxy;
and wherein the cycloalkyl and/or phenyl moieties of R¹ may carry 1, 2, 3, 4, 5 or up to the maximum number of identical or different groups R^{b} which independently of one another are selected from
R^{b} halogen, CN, nitro, C₁-C₄-alkyl, C₁-C₄-alkoxy, C₁-C₄-haloalkyl and C₁-C₄-haloalkoxy;
or two radicals R¹ bound on two adjacent carbon atoms, together with the carbon atoms to which they are bound, form a 3-, 4-, 5-, 6- or 7-membered saturated or unsaturated carbocyclic ring or a 3-, 4-, 5-, 6- or 7-membered saturated or unsaturated heterocyclic ring containing 1, 2, or 3 identical or different heteroatoms selected from O, S and N as ring members, where the carbocyclic or heterocyclic ring may carry 1, 2 or 3 substituents selected independently of one another from halogen, CN, nitro, C₁-C₄-alkyl, C₁-C₄-alkoxy, C₁-C₄-haloalkyl, and C₁-C₄-haloalkoxy;
R² represents naphthyl, 5-membered heteroaryl, or a substituent of formula Q,
wherein
the naphthyl and 5-membered heteroaryl is non-substituted or substituted by one or more group(s) selected from halogen, cyano, sulfanyl, pentafluoro-λ⁶-sulfanyl, C₁-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkoxy-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-haloalkenyl, C₂-C₈-alkynyl, C₂-C₈-haloalkynyl, C₂-C₈-alkenyloxy, C₂-C₈-haloalkenyloxy, C₃-C₈-alkynyloxy, C₃-C₈-haloalkynyloxy, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, benzyloxy, phenoxy, 4-halogen-substituted phenoxy, 4-(C₁-C₈-haloalkyl)-substituted phenoxy, benzylsulfanyl, phenylsulfanyl, or 6-membered heteroaryloxy, which is non-substituted or substituted by one or more group(s) selected from halogen and C₁-C₈-haloalkyl; and
wherein Q represents a 6-membered aromatic cycle of formula (Q-I) 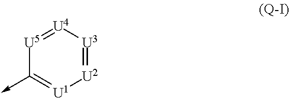
wherein
U¹ represents CX¹ or N;
U² represents CX² or N;
U³ represents CX³ or N;
U⁴ represents CX⁴ or N;
U⁵ represents CX⁵ or N;
wherein X¹, X², X³, X⁴, and X⁵ independently from each other represent hydrogen, halogen, nitro, cyano, sulfanyl, pentafluoro-λ⁶-sulfanyl, C₁-C₈-alkyl, C₃-C₈-cycloalkyl, C₃-C₇-halocycloalkyl having 1 to 5 halogen atoms, C₃-C₇-cycloalkenyl, C₁-C₈-haloalkyl having 1 to 5 halogen atoms, C₂-C₈-alkenyl, C₂-C₈-alkynyl, C₁-C₈-alkoxy, C₁-C₈-haloalkoxy having 1 to 5 halogen atoms, C₁-C₈-alkylsulfenyl, C₂-C₈-alkenyloxy, C₃-C₈-alkynyloxy, C₃-C₆-cycloalkoxy, C₁-C₈-alkylsulfinyl, C₁-C₈-alkylsulfonyl, tri(C₁-C₈-alkyl)-silyloxy, tri(C₁-C₈-alkyl)-silyl, aryl, aryloxy, arylsulfenyl, heteroaryl, heteroaryloxy,
wherein the aryl, aryloxy, arylsulfenyl, heteroaryl, heteroaryloxy is non-substituted or substituted by one or more group(s) selected from halogen, cyano, sulfanyl, pentafluoro-λ⁶-sulfanyl, C₁-C₈-alkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-alkynyl, C₂-C₈-alkenyloxy, C₂-C₈-haloalkenyloxy, C₃-C₈-alkynyloxy, C₃-C₈-haloalkynyloxy, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, 6-membered heteroaryloxy, benzyloxy, phenyloxy, benzylsulfanyl, or phenylsulfanyl,
wherein the benzyl, phenyl, 5-membered heteroaryl, 6-membered heteroaryl, 6-membered heteroaryloxy, benzyloxy, phenyloxy, benzylsulfanyl, or phenylsulfanyl is non-substituted or substituted by one or more group(s) selected from halogen, CN, nitro, C₁-C₈-alkyl, C₁-C₄-haloalkyl, C₁-C₄-alkoxy, C₁-C₄-haloalkoxy or pentafluoro-λ⁶-sulfanyl;
and wherein at most two of U¹, U², U³, U⁴ or U⁵ can represent N; or
U¹ and U² or U² and U³ or U³ and U⁴ form together an additional saturated or unsaturated 4 to 6-membered halogen- or C₁-C₈-alkyl-substituted or non-substituted ring;
R³ represents halogen, hydroxyl, cyano, isocyano, nitro, amino, sulfanyl, pentafluoro-λ⁶-sulfanyl, carboxaldehyde, hydroxycarbonyl, C₂-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-alkynyl, C₂-C₈-alkenyloxy, C₂-C₈-haloalkenyloxy, C₃-C₈-alkynyloxy, C₃-C₈-haloalkynyloxy, C₁-C₈-alkylamino, C₁-C₈-haloalkylamino, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylcarbonyl, C₁-C₈-haloalkylcarbonyl, arylcarbonyl, aryl-C₁-C₆-alkylcarbonyl, C₃-C₈-cycloalkylcarbonyl, C₃-C₈-halocycloalkylcarbonyl, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, aminothiocarbonyl, C₁-C₈-alkoxycarbonyl, C₁-C₈-haloalkoxycarbonyl, C₃-C₈-cycloalkoxycarbonyl, C₂-C₈-alkoxyalkylcarbonyl, C₂-C₈-haloalkoxyalkylcarbonyl, C₃-C₁₀-cycloalkoxyalkylcarbonyl, C₁-C₈-alkylaminocarbonyl, di-C₁-C₈-alkylaminocarbonyl, C₃-C₈-cycloalkylaminocarbonyl, C₁-C₈-alkylcarbonyloxy, C₁-C₈-haloalkylcarbonyloxy, C₃-C₈-cycloalkylcarbonyloxy, C₁-C₈-alkylcarbonylamino, C₁-C₈-haloalkylcarbonylamino, C₁-C₈-alkylaminocarbonyloxy, di-C₁-C₈-alkylaminocarbonyloxy, C₁-C₈-alkyloxycarbonyloxy, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkylaminosulfamoyl, di-C₁-C₈-alkylaminosulfamoyl, (C₁-C₈-alkoxyimino)-C₁-C₈-alkyl, (C₃-C₇-cycloalkoxyimino)-C₁-C₈-alkyl, hydroxyimino-C₁-C₈-alkyl, (C₁-C₈-alkoxyimino)-C₃-C₇-cycloalkyl, hydroxyimino-C₃-C₇-cycloalkyl, (C₁-C₈-alkylimino)-oxy, (C₁-C₈-alkylimino)-oxy-C₁-C₈-alkyl, (C₃-C₇-cycloalkylimino)-oxy-C₁-C₈-alkyl, (C₁-C₆-alkylimino)-oxy-C₃-C₇-cycloalkyl, (C₁-C₈-alkenyloxyimino)-C₁-C₈-alkyl, (C₁-C₈-alkynyloxyimino)-C₁-C₈-alkyl, (benzyloxyimino)-C₁-C₈-alkyl, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, benzyloxy, phenyloxy, benzylsulfanyl, benzylamino, phenylsulfanyl, or phenylamino, wherein the benzyl, phenyl, 5-membered heteroaryl, 6-membered heteroaryl, benzyloxy or phenyloxy is non-substituted or substituted by one or more group(s) selected from halogen, hydroxyl, cyano, isocyano, amino, sulfanyl, pentafluoro-λ⁶-sulfanyl, carboxaldehyde, hydroxycarbonyl, C₁-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-alkynyl, C₂-C₈-alkenyloxy, C₂-C₈-haloalkenyloxy, C₃-C₈-alkynyloxy, C₃-C₈-haloalkynyloxy, C₁-C₈-alkylamino, C₁-C₈-haloalkylamino, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylcarbonyl, C₁-C₈-haloalkylcarbonyl, arylcarbonyl, aryl-C₁-C₆-alkylcarbonyl, C₃-C₈-cycloalkylcarbonyl, C₃-C₈-halocycloalkylcarbonyl, C₁-C₈-alkylcarbamoyl, di-C₁-C₈-alkylcarbamoyl, N—C₁-C₈-alkyloxycarbamoyl, C₁-C₈-alkoxycarbamoyl, N—C₁-C₈-alkyl-C₁-C₈-alkoxycarbamoyl, aminothiocarbonyl, C₁-C₈-alkoxycarbonyl, C₁-C₈-haloalkoxycarbonyl, C₃-C₈-cycloalkoxycarbonyl, C₂-C₈-alkoxyalkylcarbonyl, C₂-C₈-haloalkoxyalkylcarbonyl, C₃-C₁₀-cycloalkoxyalkylcarbonyl, C₁-C₈-alkylaminocarbonyl, di-C₁-C₈-alkylaminocarbonyl, C₃-C₈-cycloalkylaminocarbonyl, C₁-C₈-alkylcarbonyloxy, C₁-C₈-haloalkylcarbonyloxy, C₃-C₈-cycloalkylcarbonyloxy, C₁-C₈-alkylcarbonylamino, C₁-C₈-haloalkylcarbonylamino, C₁-C₈-alkylaminocarbonyloxy, di-C₁-C₈-alkylaminocarbonyloxy, C₁-C₈-alkyloxycarbonyloxy, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkylaminosulfamoyl, di-C₁-C₈-alkylaminosulfamoyl, (C₁-C₈-alkoxyimino)-C₁-C₈-alkyl, (C₃-C₇-cycloalkoxyimino)-C₁-C₈-alkyl, hydroxyimino-C₁-C₈-alkyl, (C₁-C₈-alkoxyimino)-C₃-C₇-cycloalkyl, hydroxyimino-C₃-C₇-cycloalkyl, (C₁-C₈-alkylimino)-oxy, (C₁-C₈-alkylimino)-oxy-C₁-C₈-alkyl, (C₃-C₇-cycloalkylimino)-oxy-C₁-C₈-alkyl, (C₁-C₆-alkylimino)-oxy-C₃-C₇-cycloalkyl, (C₁-C₈-alkenyloxyimino)-C₁-C₈-alkyl, (C₁-C₈-alkynyloxyimino)-C₁-C₈-alkyl, (benzyloxyimino)-C₁-C₈-alkyl, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, benzyloxy, phenyloxy, benzylsulfanyl, benzylamino, phenylsulfanyl, or phenylamino;
And/or a salt and/or N-oxide thereof.

2. Imidazole derivative of formula (I) and/or a salt and/or N-oxide thereof according to claim 1, wherein
A represents a linear C₂- or C₃-alkylene bridge which may be substituted by 1, 2 or up to the maximum possible number of identical or different groups R¹, wherein each R¹ is independently selected from C₁-C₄-alkyl, C₁-C₄-haloalkyl, C₁-C₄-alkoxy, C₁-C₄-alkoxy-C₁-C₄-alkoxy and C₁-C₄-haloalkoxy, and optionally from methyl, ethyl, n-propyl, CF₃, methoxy, ethoxy and methoxymethoxy, or two substituents R¹ bound on adjacent carbon atoms, together with the carbon atoms to which they are bound, form a cyclopentyl or cyclohexyl ring; optionally represents a linear C₂- or C₃-alkylene bridge which may be substituted by 1 or 2 group(s) R¹, wherein each R¹ is independently from each other selected from C₁-C₄-alkyl and C₁-C₄-haloalkyl, optionally from methyl, ethyl, n-propyl and CF₃; optionally represents a linear C₂-alkylene bridge which may be substituted by 1 or 2 group(s) R¹, wherein each R¹ is independently from each other selected from methyl, ethyl, n-propyl and CF₃; optionally represents ethylene, 1,2-propylene, 1,2-butylene, 2,3-butylene or 1,2-pentylene.

3. Imidazole derivative of formula (I) and/or a salt and/or N-oxide thereof according to claim 1, wherein
R² represents naphthyl, thiazolyl, thienyl or a substituent of formula Q,
wherein
the naphthyl, thiazolyl or thienyl is non-substituted or substituted by one or more group(s) selected from halogen, cyano, sulfanyl, pentafluoro-λ⁶-sulfanyl, C₁-C₈-alkyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-haloalkenyl, C₂-C₈-alkynyl, C₂-C₈-haloalkynyl, C₂-C₈-alkenyloxy, C₂-C₈-haloalkenyloxy, C₃-C₈-alkynyloxy, C₃-C₈-haloalkynyloxy, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylsulfinyl, C₁-C₈-haloalkylsulfinyl, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, benzyloxy, phenoxy, 4-halogen-substituted phenoxy, 4-(C₁-C₈-haloalkyl)-substituted phenoxy, benzylsulfanyl, phenylsulfanyl, or 6-membered heteroaryloxy, which is non-substituted or substituted by one or more group(s) selected from halogen and C₁-C₈-haloalkyl; and
Q represents a 6-membered aromatic cycle of formula (Q-I) 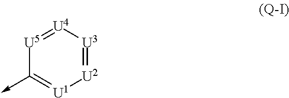
wherein
U¹ represents CX¹ or N;
U² represents CX² or N;
U³ represents CX³ or N;
U⁴ represents CX⁴ or N;
U⁵ represents CX⁵ or N; and
X¹, X², X³, X⁴ and X⁵ represent independently from each other hydrogen, halogen, pentafluoro-λ⁶-sulfanyl, C₁-C₈-alkyl, C₁-C₈-haloalkyl having 1 to 5 halogen atoms, C₁-C₈-alkoxy, C₁-C₈-haloalkoxy having 1 to 5 halogen atoms, C₁-C₈-alkylsulfanyl, C₃-C₈-cycloalkyl, C₃-C₇-halocycloalkyl having 1 to 5 halogen atoms, C₃-C₈-alkynyloxy, C₃-C₆-cycloalkoxy, aryloxy, and heteroaryloxy,
wherein the aryloxy, and heteroaryloxy is non-substituted or substituted by one or more group(s) selected from halogen, cyano, sulfanyl, pentafluoro-λ⁶-sulfanyl, C₁-C₈-haloalkyl, C₁-C₈-cyanoalkyl, C₁-C₈-alkyloxy, C₁-C₈-haloalkyloxy, tri(C₁-C₈-alkyl)silyl, tri(C₁-C₈-alkyl)silyl-C₁-C₈-alkyl, C₃-C₇-cycloalkyl, C₃-C₇-halocycloalkyl, C₃-C₇-cycloalkenyl, C₃-C₇-halocycloalkenyl, C₄-C₁₀-cycloalkylalkyl, C₄-C₁₀-halocycloalkylalkyl, C₆-C₁₂-cycloalkylcycloalkyl, C₁-C₈-alkyl-C₃-C₇-cycloalkyl, C₁-C₈-alkoxy-C₃-C₇-cycloalkyl, tri(C₁-C₈-alkyl)silyl-C₃-C₇-cycloalkyl, C₂-C₈-alkenyl, C₂-C₈-alkynyl, C₂-C₈-alkenyloxy, C₂-C₈-haloalkenyloxy, C₃-C₈-alkynyloxy, C₃-C₈-haloalkynyloxy, C₁-C₈-cyanoalkoxy, C₄-C₈-cycloalkylalkoxy, C₃-C₆-cycloalkoxy, C₁-C₈-alkylsulfanyl, C₁-C₈-haloalkylsulfanyl, C₁-C₈-alkylsulfonyl, C₁-C₈-haloalkylsulfonyloxy, C₁-C₈-alkylsulfonyl, C₁-C₈-haloalkylsulfonyl, C₁-C₈-alkylsulfonyloxy, C₁-C₈-haloalkylsulfonyloxy, C₁-C₈-alkoxyalkyl, C₁-C₈-alkylthioalkyl, C₁-C₈-alkoxyalkoxyalkyl, C₁-C₈-haloalkoxyalkyl, benzyl, phenyl, 5-membered heteroaryl, 6-membered heteroaryl, 6-membered heteroaryloxy, benzyloxy, phenyloxy, benzylsulfanyl, or phenylsulfanyl, optionally is non-substituted or substituted by one or more group(s) selected from halogen, pentafluoro-λ⁶-sulfanyl, C₁-C₈-haloalkyl, and C₁-C₈-haloalkyloxy;
and wherein at most two of U¹, U², U³, U⁴ or U⁵ can represent N.

4. Imidazole derivative of formula (I) and/or a salt and/or N-oxide thereof according to claim 1, wherein
R² represents a substituent of formula Q, wherein Q represents a phenyl or 3-pyridyl of formula (Q-I-1) or (Q-I-2) 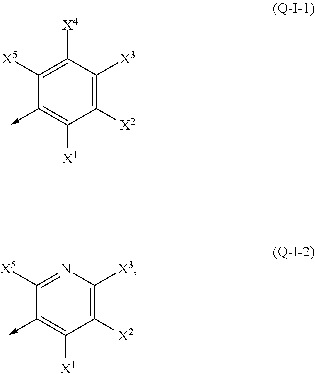
wherein
X¹ represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, optionally hydrogen, fluorine, chlorine, difluoromethyl or trifluoromethyl;
X² represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, optionally hydrogen;
X³ represents 4-fluorophenoxy, 4-chlorophenoxy, 4-bromophenoxy, 4-iodophenoxy, 4-(trifluoromethyl)phenoxy or pyridin-3-yloxy, wherein pyridin-3-yloxy is substituted in 5- or 6-position by one group selected from fluorine, chlorine, bromine, iodine and trifluoromethyl;
X⁴ represents hydrogen, fluorine, methyl, ethyl, difluoromethyl, trifluoromethyl, methoxy, trifluoromethoxy, chlorodifluoromethoxy, 1,1,2,2-tetrafluoroethoxy, or methylsulfenyl, optionally hydrogen; and
X⁵ represents hydrogen, fluorine, chlorine, bromine, methyl, ethyl, difluoromethyl, trifluoromethyl, methoxy, trifluoromethoxy, chlorodifluoromethoxy, 1,1,2,2-tetrafluoroethoxy, or methylsulfenyl, optionally hydrogen, fluorine, chlorine, difluoromethyl or trifluoromethyl.

5. Imidazole derivative of formula (I) and/or a salt and/or N-oxide thereof according to claim 1, wherein
R³ represents halogen, cyano, carboxaldehyde, hydroxycarbonyl, C₂-C₄-alkyl, C₁-C₄-haloalkyl, C₁-C₄-cyanoalkyl, C₁-C₄-alkyloxy, C₁-C₄-haloalkyloxy, C₃-C₇-cycloalkyl, C₃-C₇-halocycloalkyl, C₂-C₅-alkenyl, C₂-C₅-alkynyl, C₁-C₄-alkylsulfanyl, C₁-C₄-haloalkylsulfanyl, C₁-C₄-alkylcarbonyl, C₁-C₄-haloalkylcarbonyl, aminothiocarbonyl, C₁-C₄-alkoxycarbonyl, C₁-C₄-haloalkoxycarbonyl, benzyl, phenyl, furyl, pyrrolyl, thienyl, pyridyl, benzyloxy, or phenyloxy, wherein the benzyl, phenyl, 5-membered heteroaryl, 6-membered heteroaryl, benzyloxy or phenyloxy may be optionally substituted by one or more group(s) selected from halogen, C₁-C₈-alkyl, C₁-C₈-haloalkyl, C₁-C₈-alkyloxy, C₁-C₈-haloalkyloxy; optionally represents fluorine, chlorine, bromine, iodine, cyano, hydroxycarbonyl, carboxaldehyde, trifluoromethyl, cyanomethyl, methoxy, methylsulfanyl, cyclopropyl, ethinyl, methylcarbonyl (acetyl), carboxyl, aminothiocarbonyl, methoxycarbonyl, ethoxycarbonyl, phenyl, or 2-thienyl, optionally fluorine, chlorine, bromine, iodine, or cyano.

6. Imidazole derivative of formula (I) and/or a salt and/or N-oxide thereof according to claim 1, wherein
A represents ethylene, 1,2-propylene, 1,2-butylene, 2,3-butylene or 1,2-pentylene;
R² represents a substituent of formula Q, wherein Q represents a phenyl or 3-pyridyl of formula (Q-I-1) or (Q-I-2) 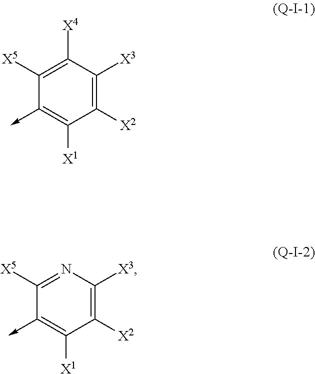
wherein
X¹ represents hydrogen, fluorine, chlorine, difluoromethyl or trifluoromethyl;
X² represents hydrogen;
X³ represents 4-fluorophenoxy, 4-chlorophenoxy, 4-bromophenoxy, 4-iodophenoxy, 4-(trifluoromethyl)phenoxy or pyridin-3-yloxy, wherein the pyridin-3-yloxy is substituted in 5- or 6-position by one group selected from fluorine, chlorine, bromine, iodine and trifluoromethyl;
X⁴ represents hydrogen;
X⁵ represents hydrogen, fluorine, chlorine, difluoromethyl or trifluoromethyl; and
R³ represents fluorine, chlorine, bromine, cyano, trifluoromethyl, or ethinyl.

7. Method for controlling one or more harmful microorganisms in crop protection and/or in protection of materials, comprising applying at least one compound of formula (I) and/or a salt and/or N-oxide thereof according to claim 1 to the harmful microorganisms and/or a habitat thereof.

8. Method for controlling one or more phytopathogenic harmful fungi in crop protection and/or in protection of materials, comprising applying at least one compound of formula (I) and/or a salt and/or N-oxide thereof according to claim 1 to the phytopathogenic harmful fungi and/or a habitat thereof.

9. Composition for controlling one or more harmful microorganisms, optionally for controlling phytopathogenic harmful fungi, comprising a content of at least one compound of formula (I) and/or a salt and/or N-oxide thereof according to claim 1, in addition to at least one extender and/or surfactant.

10. Composition according to claim 9 comprising at least one further active ingredient selected from the group of insecticides, attractants, sterilants, bactericides, acaricides, nematicides, fungicides, growth regulators, herbicides, fertilizers, safeners and semiochemicals.

11. Process for producing a composition according to claim 9, comprising mixing at least one compound of formula (I) and/or a salt and/or N-oxide thereof according to claim 1 with at least one extender and/or surfactant.

12. A product comprising at least one compound of formula (I) and/or a salt and/or N-oxide thereof according to claim 1 for control of one or more harmful microorganisms, optionally phytopathogenic harmful fungi, in crop protection and/or in protection of materials.

13. A product comprising at least one compound of formula (I) and/or a salt and/or N-oxide thereof according to claim 1 for treatment of a transgenic plant or a seed, optionally a seed of a transgenic plant.
